SEQUENCE LISTING (179 sequences)

SEQ ID NO: 1 - protein, 191 aa, Arabidopsis thaliana
  thale cress PYR/PYL receptor, Pyrabactin resistance 1, abscisic acid receptor PYR1 (PYR1), ABI1-binding protein 6 (ABIP6), regulatory components of ABA receptor 11 (RCAR11), At4g17870, T6K21.50
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 2 - protein, 221 aa, Arabidopsis thaliana
  thale cress PYR/PYL receptor, abscisic acid receptor PYL1, PYR1-like protein 1 (PYL1), ABI1-binding protein 6 (ABIP6), regulatory components of ABA receptor 9 (RCAR12), At5g46790, MZA15.21
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 3 - protein, 190 aa, Arabidopsis thaliana
  thale cress PYR/PYL receptor, abscisic acid receptor PYL2, PYR1-like protein 2 (PYL2), ABI1-binding protein 6 (ABIP6), regulatory components of ABA receptor 14 (RCAR14), Bet v I allergen family protein, At2g26040, T19L18.15
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 4 - protein, 209 aa, Arabidopsis thaliana
  thale cress PYR/PYL receptor, abscisic acid receptor PYL3, PYR1-like protein 3 (PYL3), regulatory components of ABA receptor 13 (RCAR13), At1g73000, F3N23.20
MNLAPIHDPSSSSTTTTSSSTPYGLTKDEFSTLDSIIRTHHTFPRSPNTCTSLIAHRVDAPAHAIWRFVRDFANPNKYKHFIKSCTIRVNGNGIKEIKVGTIREVSVVSGLPASTSVEILEVLDEEKRILSFRVLGGEHRLNNYRSVTSVNEFVVLEKDKKKRVYSVVLESYIVDIPQGNTEEDTRMFVDTVVKSNLQNLAVISTASPT

SEQ ID NO: 5 - protein, 207 aa, Arabidopsis thaliana
  thale cress PYR/PYL receptor, abscisic acid receptor PYL4, PYR1-like protein 4 (PYL4), ABI1-binding protein 2 (ABIP2), regulatory components of ABA receptor 10 (RCAR10), At2g38310, T19C21.20
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 6 - protein, 203 aa, Arabidopsis thaliana
  thale cress PYR/PYL receptor, abscisic acid receptor PYL5, PYR1-like protein 5 (PYL5), ABI1-binding protein 3 (ABIP3), regulatory components of ABA receptor 8 (RCAR8), Bet v I allergen family protein, At5g05440, K18I23.25
MRSPVQLQHGSDATNGFHTLQPHDQTDGPIKRVCLTRGMHVPEHVAMHHTHDVGPDQCCSSVVQMIHAPPESVWALVRRFDNPKVYKNFIRQCRIVQGDGLHVGDLREVMVVSGLPAVSSTERLEILDEERHVISFSVVGGDHRLKNYRSVTTLHASDDEGTVVVESYIVDVPPGNTEEETLSFVDTIVRCNLQSLARSTNRQ

SEQ ID NO: 7 - protein, 215 aa, Arabidopsis thaliana
  thale cress PYR/PYL receptor, abscisic acid receptor PYL6, PYR1-like protein 6 (PYL6), ABI1-binding protein 5 (ABIP5), regulatory components of ABA receptor 9 (RCAR9), Bet v I allergen family protein, At2g40330, T7M7.15
MPTSIQFQRSSTAAEAANATVRNYPHHHQKQVQKVSLTRGMADVPEHVELSHTHVVGPSQCFSVVVQDVEAPVSTVWSILSRFEHPQAYKHFVKSCHVVIGDGREVGSVREVRVVSGLPAAFSLERLEIMDDDRHVISFSVVGGDHRLMNYKSVTTVHESEEDSDGKKRTRVVESYVVDVPAGNDKEETCSFADTIVRCNLQSLAKLAENTSKFS

SEQ ID NO: 8 - protein, 211 aa, Arabidopsis thaliana
  thale cress PYR/PYL receptor, abscisic acid receptor PYL7, PYR1-like protein 7 (PYL7), ABI1-binding protein 7 (ABIP7), regulatory components of ABA receptor 2 (RCAR2), At4g01026
MEMIGGDDTDTEMYGALVTAQSLRLRHLHHCRENQCTSVLVKYIQAPVHLVWSLVRRFDQPQKYKPFISRCTVNGDPEIGCLREVNVKSGLPATTSTERLEQLDDEEHILGINIIGGDHRLKNYSSILTVHPEMIDGRSGTMVMESFVVDVPQGNTKDDTCYFVESLIKCNLKSLACVSERLAAQDITNSIATFCNASNGYREKNHTETNL

SEQ ID NO: 9 - protein, 188 aa, Arabidopsis thaliana
  thale cress PYR/PYL receptor, abscisic acid receptor PYL8, PYR1-like protein 8 (PYL8), ABI1-binding protein 1 (ABIP1), regulatory components of ABA receptor 3 (RCAR3), At5g53160, MFH8.10
MEANGIENLTNPNQEREFIRRHHKHELVDNQCSSTLVKHINAPVHIVWSLVRRFDQPQKYKPFISRCVVKGNMEIGTVREVDVKSGLPATRSTERLELLDDNEHILSIRIVGGDHRLKNYSSIISLHPETIEGRIGTLVIESFVVDVPEGNTKDETCYFVEALIKCNLKSLADISERLAVQDTTESRV

SEQ ID NO: 10 - protein, 187 aa, Arabidopsis thaliana
  thale cress PYR/PYL receptor, abscisic acid receptor PYL9, PYR1-like protein 9 (PYL9), ABI1-binding protein 4 (ABIP4), regulatory components of ABA receptor 1 (RCAR1), At1g01360, F6F3.16
MMDGVEGGTAMYGGLETVQYVRTHHQHLCRENQCTSALVKHIKAPLHLVWSLVRRFDQPQKYKPFVSRCTVIGDPEIGSLREVNVKSGLPATTSTERLELLDDEEHILGIKIIGGDHRLKNYSSILTVHPEIIEGRAGTMVIESFVVDVPQGNTKDETCYFVEALIRCNLKSLADVSERLASQDITQ

SEQ ID NO: 11 - protein, 183 aa, Arabidopsis thaliana
  thale cress PYR/PYL receptor, abscisic acid receptor PYL10, PYR1-like protein 10 (PYL10), ABI1-binding protein 8 (ABIP8), regulatory components of ABA receptor 4 (RCAR4), At4g27920, T13J8.30
MNGDETKKVESEYIKKHHRHELVESQCSSTLVKHIKAPLHLVWSIVRRFDEPQKYKPFISRCVVQGKKLEVGSVREVDLKSGLPATKSTEVLEILDDNEHILGIRIVGGDHRLKNYSSTISLHSETIDGKTGTLAIESFVVDVPEGNTKEETCFFVEALIQCNLNSLADVTERLQAESMEKKI

SEQ ID NO: 12 - protein, 161 aa, Arabidopsis thaliana
  thale cress PYR/PYL receptor, abscisic acid receptor PYL11, PYR1-like protein 11 (PYL11), regulatory components of ABA receptor 5 (RCAR5), Bet v I allergen family protein, At5g45860, K15I22.6
METSQKYHTCGSTLVQTIDAPLSLVWSILRRFDNPQAYKQFVKTCNLSSGDGGEGSVREVTVVSGLPAEFSRERLDELDDESHVMMISIIGGDHRLVNYRSKTMAFVAADTEEKTVVVESYVVDVPEGNSEEETTSFADTIVGFNLKSLAKLSERVAHLKL

SEQ ID NO: 13 - protein, 159 aa, Arabidopsis thaliana
  thale cress PYR/PYL receptor, abscisic acid receptor PYL12, PYR1-like protein 12 (PYL12), regulatory components of ABA receptor 6 (RCAR6), Bet v I allergen family protein, At5g45870, K15I22.7
MKTSQEQHVCGSTVVQTINAPLPLVWSILRRFDNPKTFKHFVKTCKLRSGDGGEGSVREVTVVSDLPASFSLERLDELDDESHVMVISIIGGDHRLVNYQSKTTVFVAAEEEKTVVVESYVVDVPEGNTEEETTLFADTIVGCNLRSLAKLSEKMMELT

SEQ ID NO: 14 - protein, 164 aa, Arabidopsis thaliana
  thale cress PYR/PYL receptor, abscisic acid receptor PYL13, PYR1-like protein 13 (PYL13), regulatory components of ABA receptor 7 (RCAR7), At4g18620, F28A21.30
MESSKQKRCRSSVVETIEAPLPLVWSILRSFDKPQAYQRFVKSCTMRSGGGGGKGGEGKGSVRDVTLVSGFPADFSTERLEELDDESHVMVVSIIGGNHRLVNYKSKTKVVASPEDMAKKTVVVESYVVDVPEGTSEEDTIFFVDNIIRYNLTSLAKLTKKMMK

SEQ ID NO: 15 - protein, 191 aa, Brassica oleracea
  wild cabbage Streptomyces cyclase/dehydrase family protein, locus tag 40.t00062, GenBank Accession No. ABD65175.1
MPSQLTPEERSELAQSIAEFHTYHLGPGSCSSLHAQRIHAPPEIVWSVVRRFDKPQTYKHFIKSCSVEDGFEMRVGCTRAVNVISGLPANTSTERLDILDDERRVTGFSIIGGEHRLTNYKSVTTVHRFEKERRIWTVVLESYVVDMPEGNSEDDTRMFADTVVKLNLQKLATVTEAMARNAGDGSGAQVT

SEQ ID NO: 16 - protein, 281 aa, Brassica oleracea
  wild cabbage Streptomyces cyclase/dehydrase family protein, locus tag 23.t00047, GenBank Accession No. ABD65631.1
MPSELTQEERSKLTQSISEFHTYHLGPGSCSSLHAQRIHAPPEIVWSVVRQFDKPQTYKHFIKSCSVEEGFEMRVGCTRDVIVISGLPANTSTERLDMLDDERRVTGFSIIGGEHRLKNYKSVTTVHRFERERRIWTVVLESYVVDMPEGNSEDDTRMFADTVVKLNLQKLATVTEAMARNAGDGRGSRETTCRESFHLITAFEKQRQITEPTVYQNPPYHTGMTPEPRTSTVFIELEDHRTLPGNLTPTTEEHLQRMYQRFWGIRQLQRPRQSFGERQSI

SEQ ID NO: 17 - protein, 453 aa, Vitis vinifera
  wine grape cultivar PN40024 unnamed protein product, locus tag GSVIVT00015766001, GenBank Accession No. CAO63410.1
MQMKYLEGKQNLMEEKGEKQCIPMDLAVREAQFKGSLLDRITWLEQRLHKLSLQLETRSKQQPHPSRMQTAGETSSRHGPKKELSCSFPVFSTRNHNHGHKQTSQFHVPRFEYQEGGRENPAVVITKLTPFHHPKIITILFPISNYFIIFFFLTFDTKKQYPLLFPILPSRFLPISHLITQEIEKYKTSSHFSSPASLFAAMNKAETSSMAEAESEDSETTTPTTHHLTIPPGLTQPEFQELAHSISEFHTYQVGPGQCSSLLAQRVHAPLPTVWSVVRRFDKPQTYKHFIKSCHVEDGFEMRVGCLRDVNVISGLPAETSTERLDILDDERHVTGFSIIGGEHRLRNYRSVTTNHGGEIWTVVLESYVVDMPEGNTEEDTRLFADTVVKLNLQKLASVTEVSQSCNYPCQFHIIENEDIQPEEMNLGVLTTSIEEQRKKKRVVAMKDGSTSS

SEQ ID NO: 18 - protein, 195 aa, Vitis vinifera
  wine grape cultivar Pinot Noir hypothetical protein, clone ENTAV 115, locus tag VITISV_033963, GenBank Accession No. CAN64657.1
MAEAESEDSETTTPTTHHLTIPPGLTQPEFQELAHSISEFHTYQVGPGQCSSLLAQRVHAPLPTVWSVVRRFDKPQTYKHFIKSCHVEDGFEMRVGCLRDVNVISGLPAETSTERLDILDDERHVTGFSIIGGEHRLRNYRSVTTVHEYQNHGGEIWTVVLESYVVDMPEGNTEEDTRLFADTVVKLNLSEAXRR

SEQ ID NO: 19 - protein, 217 aa, Medicago truncatula
  barrel medic unknown protein, clone MTYFD_FE_FF_FG1G-N-24, GenBank Accession No. ACJ85026.1
MEKAESSTASTSDQDSDENHRTQHHLTLPSGLRQHEFDSLIPFINSHHTYLIGPNQCSTLLAQRIHAPPQTVWSVVRSFDKPQIYKHIIKSCSLKEGFQMKVGCTRDVNVISGLPAATSTERLDVLDDERRVTGFSIIGGEHRLKNYRSVTSVHGFGDGDNGGEIWTVVLESYVVDVPEGNTEEDTRLFADTVVKLNLQKLASVTEGKNRDGDGKSH

SEQ ID NO: 20 - protein, 212 aa, Oryza sativa
  rice Japonica Group, cultivar Nipponbare, conserved hypothetical protein Os10g0573400, GenBank Accession No. NP_00106570.1
MEQQEEVPPPPAGLGLTAEEYAQVRATVEAHHRYAVGPGQCSSLLAQRIHAPPAAVWAVVRRFDCPQVYKHFIRSCVLRPDPHHDDNGNDLRPGRLREVSVISGLPASTSTERLDLLDDAHRVFGFTITGGEHRLRNYRSVTTVSQLDEICTLVLESYIVDVPDGNTEDDTRLFADTVIRLNLQKLKSVSEANANAAAAAAAPPPPPPAAAE

SEQ ID NO: 21 - protein, 212 aa, Zea mays
  maize cyclase/dehydrase family protein, clone 306819, GenBank Accession No. ACG40002.1
MDQQGAGGDAEVPAGLGLTAAEYEQLRSTVDAHHRYAVGEGQCSSLLAQRIHAPPEAVWAVVRRFDCPQVYKHFIRSCALRPDPEAGDALCPGRLREVSVISGLPASTSTERLDLLDDAARVFGFSITGGEHRLRNYRSVTTVSELAVPAICTVVLESYVVDVPDGNTEDDTRLFADTVIRLNLQKLKSVAEANAAEAAATTNSVLLPRPAE

SEQ ID NO: 22 - protein, 212 aa, Zea mays
  maize cyclase/dehydrase family protein, clone 241996, GenBank Accession No. ACG34473.1
MDQQGAGGDAXVPAGLGLTAAEYEQLRSTVDAHHRYAVGEGQCSSLLAQRIHAPPEAVWAVVRRFDCPQVYKHFIRSCALRPDPEAGDALCPGRLREVSVISGLPASTSTERLDLLDDAARVFGFSITGGEHRLRNYRSVTTVSELADPAICTVVLESYVVDVPDGNTEDDTRLFADTVIRLNLQKLKSVTEANAAEAAATTNSVLLPRPAE

SEQ ID NO: 23 - protein, 233 aa, Vitis vinifera
  wine grape cultivar PN40024 unnamed protein product, locus tag GSVIVT00032173001, GenBank Accession No. CAO43790.1
MDPHHHHGLTEEEFRALEPIIQNYHTFEPSPNTCTSLITQKIDAPAQVVWPFVRSFENPQKYKHFIKDCTMRGDGGVGSIREVTVVSGLPASTSTERLEILDDEKHILSFRVVGGEHRLNNYRSVTSVNDFSKEGKDYTIVLESYIVDIPEGNTGEDTKMFVDTVVKLNLQKLAVVAITSLHENEEIADNEGPSREISLQSETESAERGDERRDGDGPSKACNRNEWHCTTKE

SEQ ID NO: 24 - protein, 207 aa, Oryza sativa
  rice Japonica Group, cultivar Nipponbare, Bet v I allergen-like protein, clone P0495C02.29, GenBank Accession No. BAD25659.1
MEPHMERALREAVASEAERRELEGVVRAHHTFPAAERAAGPGRRPTCTSLVAQRVDAPLAAVWPIVRGFANPQRYKHFIKSCELAAGDGATVGSVREVAVVSGLPASTSTERLEILDDDRHVLSFRVVGGDHRLRNYRSVTSVTEFSSPSSPPRPYCVVVESYVVDVPEGNTEEDTRMFTDTVVKLNLQKLAAVATSSSPPAAGNHH

SEQ ID NO: 25 - protein, 210 aa, Oryza sativa
  rice Indica Group, cultivar 93-11, hypothetical protein OsI_06433, GenBank Accession No. EAY85077.1
MEPHMERALREAVASEAERRELEGVVRAHHTFPAAERAAGPGRRPTCTSLVAQRVDAPLAAVWPIVRGFANPQRYKHFIKSCELAAGDGATVGSVREVAVVSGLPASTSTERLEILDDDRHVLSFRVVGGDHRLRNYRSVTSVTEFSSPSSPPSPPRPYCVVVESYVVDVPEGNTEEDTRMFTDTVVKLNLQKLAAVATSSSPPAAGNHH

SEQ ID NO: 26 - protein, 200 aa, Zea mays
  maize strain B73 unknown protein, clone ZM_BFb0151H07, GenBank Accession No. ACF82013.1
MPYTAPRPSPQQHSRVLSGGGAKAASHGASCAAVPAEVARHHEHAARAGQCCSAVVQAIAAPVGAVWSVVRRFDRPQAYKHFIRSCRLVGGGDVAVGSVREVRVVSGLPATSSRERLEILDDERRVLSFRVVGGEHRLANYRSVTTVHEAGAGAGTGTVVVESYVVDVPHGNTADETRVFVDTIVRCNLQSLARTAERLA

SEQ ID NO: 27 - protein, 215 aa, Vitis vinifera
  wine grape cultivar PN40024 unnamed protein product, locus tag GSVIVT00037390001, GenBank Accession No. CAO48777.1
MPSNPPKSSLVVHRINSPNSITTATTASAAANNHNTSTMPPHKQVPDAVSRHHTHVVGPNQCCSAVVQQIAAPVSTVWSVVRRFDNPQAYKHFVKSCHVVVGDGDVGTLREVHVISGLPAANSTERLEILDDERHVLSFSVIGGDHRLSNYRSVTTLHPSPSSTGTVVLESYVVDIPPGNTKEDTCVFVDTIVRCNLQSLAQIAENAAGCKRSSS

SEQ ID NO: 28 - protein, 213 aa, Nicotiana tabacum
  tobacco hypothetical protein, gene c17, GenBank Accession No. CAI84653.1
MPPSSPDSSVLLQRISSNTTPDFACKQSQQLQRRTMPIPCTTQVPDSVVRFHTHPVGPNQCCSAVIQRISAPVSTVWSVVRRFDNPQAYKHFVKSCHVIVGDGDVGTLREVRVISGLPAASSTERLEILDDERHVISFSVVGGDHRLANYRSVTTLHPEPSGDGTTIVVESYVVDVPPGNTRDETCVFVDTIVKCNLTSLSQIAVNVNRRKDS

SEQ ID NO: 29 - protein, 208 aa, Oryza sativa
  rice Indica Group, cultivar 93-11, hypothetical protein OsI_04285, GenBank Accession No. EAY76350.1
MPYAAVRPSPPPQLSRPIGSGAGGGKACPAVPCEVARYHEHAVGAGQCCSTVVQAIAAPADAVWSVVRRFDRPQAYKKFIKSCRLVDGDGGEVGSVREVRVVSGLPATSSRERLEVLDDDRRVLSFRIVGGEHRLANYRSVTTVHEAAAPAMAVVVESYVVDVPPGNTWEETRVFVDTIVRCNLQSLARTVERLAPEAPRANGSIDHA

SEQ ID NO: 30 - protein, 208 aa, Oryza sativa
  rice Japonica Group, cultivar Nipponbare, Bet v I allergen-like protein, gene B1088C09.11, clone B1088C09, GenBank Accession No. BAB68102.1
MPYAAVRPSPPPQLSRPIGSGAGGGKACPAVPCEVARYHEHAVGAGQCFSTVVQAIAAPADAVWSVVRRFDRPQAYKKFIKSCRLVDGDGGEVGSVREVRVVSGLPATSSRERLEVLDDDRRVLSFRIVGGEHRLANYRSVTTVHEAAAPAMAVVVESYVVDVPPGNTWEETRVFVDTIVRCNLQSLARTVERLAPEAPRANGSIDHA

SEQ ID NO: 31 - protein, 213 aa, Picea sitchensis
  Sitka spruce cultivar FB3-425, unknown protein, clone WS0276_P02, GenBank Accession No. ABK22940.1
MDIIAGFDQLSFRLSGASKQITKTGAVQYLKGEEGYGEWLKEVMGRYHYHSHDGARECRCSSVVVQQVEAPVSVVWSLVRRFDQPQVYKHFVSNCFMRGDLKVGCLREVRVVSGLPAATSTERLDILDEERHILSFSIVGGDHRLNNYRSITTLHETLINGKPGTIVIESYVLDVPHGNTKEETCLFVDTIVKCNLQSLAHVSNHLNSTHRCL

SEQ ID NO: 32 - protein, 207 aa, Oryza sativa
  rice Japonica Group, cultivar Nipponbare, hypothetical protein Os06g0562200, Bet v I allergen family protein, GenBank Accession No. NP_001057874.1
MEAHVERALREGLTEEERAALEPAVMAHHTFPPSTTTATTAAATCTSLVTQRVAAPVRAVWPIVRSFGNPQRYKHFVRTCALAAGDGASVGSVREVTVVSGLPASTSTERLEMLDDDRHIISFRVVGGQHRLRNYRSVTSVTEFQPPAAGPGPAPPYCVVVESYVVDVPDGNTAEDTRMFTDTVVKLNLQMLAAVAEDSSSASRRRD

SEQ ID NO: 33 - protein, 216 aa, Oryza sativa
  rice Japonica Group, cultivar Nipponbare, hypothetical protein Os05g0473000, Streptomyces cyclase/dehydrase family protein, GenBank Accession No. NP_001055819.1
MPYTAPRPSPPQHSRIGGCGGGGVLKAAGAAGHAASCVAVPAEVARHHEHAAGVGQCCSAVVQAIAAPVDAVWSVVRRFDRPQAYKHFIRSCRLLDGDGDGGAVAVGSVREVRVVSGLPATSSRERLEILDDERRVLSFRVVGGEHRLSNYRSVTTVHETAAGAAAAVVVESYVVDVPHGNTADETRMFVDTIVRCNLQSLARTAEQLALAAPRAA

SEQ ID NO: 34 - protein, 212 aa, Vitis vinifera
  wine grape cultivar PN40024 unnamed protein product, locus tag GSVIVT00029365001, GenBank Accession No. CAO41436.1
MPSSLQLHRINNIDPTTVAVAATAAVNCHKQSRTPLRCATPVPDAVASYHAHAVGPHQCCSMVVQTTAAALPTVWSVVRRFDNPQAYKHFLKSCHVIFGDGDIGTLREVHVVSGLPAESSTERLEILDDERHVLSFSVVGGDHRLCNYRSVTTLHPSPTGTGTVVVESYVVDIPPGNTKEDTCVFVDTIVKCNLQSLAQMSEKLTNNNRNSS

SEQ ID NO: 35 - protein, 218 aa, Zea mays
  maize cyclase/dehydrase family protein, clone 1678999, GenBank Accession No. ACG30334.1
MPCLQASSPGSMPYQHHGRGVGCAAEAGAAVGASAGTGTRCGAHDGEVPAEAARHHEHAAPGPGRCCSAVVQRVAAPAEAVWSVVRRFDQPQAYKRFVRSCALLAGDGGVGTLREVRVVSGLPAASSRERLEVLDDESHVLSFRVVGGEHRLQNYLSVTTVHPSPAAPDAATVVVESYVVDVPPGNTPEDTRVFVDTIVKCNLQSLATTAEKLALAAV

SEQ ID NO: 36 - protein, 179 aa, Physcomitrella patens
  Physcomitrella patens subsp. patens moss, ecotype Gransden 2004, hypothetical protein, predicted protein, locus tag PHYPADRAFT_222359, GenBank Accession No. XP_001778048.1
MQTKGRQADFQTLLEGQQDLICRFHRHELQPHQCGSILLQLIKAPVETVWSVARSFDKPQVYKRFIQTCEIIEGDGGVGSIREVRLVSSIPATSSIERLEILDDEEHIISFRVLGGGHRLQNYWSVTSLHSHEIDGQMGTLVLESYVVDIPEGNTREETHMFVDTVVRCNLKALAQVSE

SEQ ID NO: 37 - protein, 229 aa, Oryza sativa
  rice Indica Group, cultivar 93-11, hypothetical protein OsI_11160, GenBank Accession No. EAY89631.1
MPCIPASSPGIPHQHQHQHHRALAGVGMAVGCAAEAAVAAAGVAGTRCGAHDGEVPMEVARHHEHAEPGSGRCCSAVVQHVAAPAPAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 38 - protein, 229 aa, Oryza sativa
  rice Japonica Group, cultivar Nipponbare, hypothetical protein Os03g0297600, Streptomyces cyclase/dehydrase family protein, GenBank Accession No. NP_001049838.1
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 39 - protein, 205 aa, Medicago truncatula
  barrel medic unknown protein, clone MTYFP_FQ_FR_FS1G-H-19, GenBank Accession No. ACJ85898.1
MPSPVQFQRFDSNTAITNGVNCPKQIQACRYALSSLKPTVSVPETVVDHHMHVVGQNQCYSVVIQTINASVSTVWSVVRRFDYPQGYKHFVKSCNVVASGDGIRVGALREVRLVSGLPAVSSTERLDILDEERHVISFSVVGGVHRCRNYRSVTTLHGDGNGGTVVIESYVVDVPQGNTKEETCSFADTIVRCNLQSLVQIAEKL

SEQ ID NO: 40 - protein, 212 aa, Zea mays
  maize AT-rich element binding factor 3, clone 1458362, GenBank Accession No. ACG26321.1
MPFAASRTSQQQHSRVATNGRAVAVCAGHAGVPDEVARHHEHAVAAGQCCAAMVQSIAAPVDAVWSLVRRFDQPQRYKRFIRSCHLVDGDGAEVGSVRELLLVSGLPAESSRERLEIRDDERRVISFRVLGGDHRLANYRSVTTVHEAAPSQDGRPLTMVVESYVVDVPPGNTVEETRIFVDTIVRCNLQSLEGTVIRQLEIAAMPHDDNQN

SEQ ID NO: 41 - protein, 233 aa, Zea mays
  maize strain B73 unknown protein, clone ZM_BFb0105O18, GenBank Accession No. ACF87013.1
MRERNSSIDQEHQRGSSSRSTMPFAASRTSQQQHSRVATNGRAVAVCAGHAGVPDEVARHHEHAVAAGQCCAAMVQSIAAPVDAVWSLVRRFDQPQRYKRFIRSCHLVDGDGAEVGSVRELLLVSGLPAESSRERLEIRDDERRVISFRVLGGDHRLANYRSVTTVHEAAPSQDGRPLTMVVESYVVDVPPGNTVEETRIFVDTIVRCNLQSLEGTVIRQLEIAAMPHDDNQN

SEQ ID NO: 42 - protein, 194 aa, Physcomitrella patens
  Physcomitrella patens subsp. patens moss, ecotype Gransden 2004, hypothetical protein, predicted protein, locus tag PHYPADRAFT_209242, GenBank Accession No. XP_001762113.1
MMQEKQGRPDFQFLLEGQQDLICRFHKHELLPHQCGSILLQQIKAPVQTVWLIVRRFDEPQVYKRFIQRCDIVEGDGVVGSIREVQLVSSIPATSSIERLEILDDEEHIISFRVLGGGHRLQNYWSVTSLHRHEIQGQMGTLVLESYVVDIPDGNTREETHTFVDTVVRCNLKALAQVSEQKHLLNSNEKPAAP

SEQ ID NO: 43 - protein, 191 aa, Vitis vinifera
  wine grape cultivar PN40024 unnamed protein product, locus tag GSVIVT00035869001, GenBank Accession No. CAO48052.1
MKVYSPSQILAERGPRAQAMGNLYHTHHLLPNQCSSLVVQTTDAPLPQVWSMVRRFDRPQSYKRFVRGCTLRRGKGGVGSVREVNIVSGLPAEISLERLDKLDDDLHVMRFTVIGGDHRLANYHSTLTLHEDEEDGVRKTVVMESYVVDVPGGNSAGETCYFANTIIGFNLKALAAVTETMALKANIPSGF

SEQ ID NO: 44 - protein, 217 aa, Physcomitrella patens
  Physcomitrella patens subsp. patens moss, ecotype Gransden 2004, hypothetical protein, predicted protein, locus tag PHYPADRAFT_132509, GenBank Accession No. XP_001767821.1
MQQVKGRQDFQRLLEAQQDLICRYHTHELKAHQCGSILLQQIKVPLPIVWAIVRSFDKPQVYKRFIQTCKITEGDGGVGSIREVHLVSSVPATCSIERLEILDDEKHIISFRVLGGGHRLQNYSSVSSLHELEVEGHPCTLVLESYMVDIPDGNTREETHMFVDTVVRCNLKSLAQISEQQYNKDCLQQKQHDQQQMYQQRHPPLPPIPITDKNMER

SEQ ID NO: 45 - protein, 195 aa, Physcomitrella patens
  Physcomitrella patens subsp. patens moss, ecotype Gransden 2004, hypothetical protein, predicted protein, locus tag PHYPADRAFT_213389, GenBank Accession No. XP_001767012.1
MRFDIGHNDVRGFFTCEEEHAYALHSQTVELNQCGSILMQQIHAPIEVVWSIVRSFGSPQIYKKFIQACILTVGDGGVGSIREVFLVSGVPATSSIERLEILDDEKHVFSFRVLKGGHRLQNYRSVTTLHEQEVNGRQTTTVLESYVVDVPDGNTREETHMFADTVVMCNLKSLAQVAEWRAMQGITQQLSTSSL

SEQ ID NO: 46 - protein, 172 aa, Vitis vinifera
  wine grape cultivar Pinot Noir hypothetical protein, clone ENTAV 115, locus tag VITISV_004947, GenBank Accession No. CAN72620.1
MGNLYHTHHLLPNQCSSLVVQTTDAPLPQVWSMVRRFDRPQSYKRFVRGCTLRRGKGGVGSVREVNIVSGLPAEISLERLDKLDDDLHVMRFTVIGGDHRLANYHSTLTLHEDEEDGVRKTVVMESYVVDVPGGNSAGETCYFANTIIGFNLKALAAVTETMALKANIPSGF

SEQ ID NO: 47 - protein, 196 aa, Picea sitchensis
  Sitka spruce cultivar FB3-425, unknown protein, clone WS0281_I24, GenBank Accession No. ABK23752.1
MEDLSSWREGRAMWLGNPPSESELVCRHHRHELQGNQCSSFLVKHIRAPVHLVWSIVRTFDQPQKYKPFVHSCSVRGGITVGSIRNVNVKSGLPATASEERLEILDDNEHVFSIKILGGDHRLQNYSSIITVHPEIIDGRPGTLVIESYVVDVPEGNTREETRFFVEALVKCNLKSLADVSERLASQHHTELLERT

SEQ ID NO: 48 - protein, 185 aa, Solanum tuberosum
  potato cultivar Kuras, CAPIP1-like protein, clone 153D02, similar to Casicum annuum CAPIP1, GenBank Accession No. ABB29920.1
MNANGFCGVEKEYIRKHHLHEPKENQCSSFLVKHIRAPVHLVWSLVRRFDQPQKYKPFISRCIVQGDLEIGSLREVDVKSGLPATTSTERLELLDDEEHILSVRIVGGDHRLRNYSSVISVHPEVIDGRPGTVVLESFVVDVPEGNTKDETCYFVEALINCNLKSLADISERVAVQDRTEPIDQV

SEQ ID NO: 49 - protein, 190 aa, Medicago truncatula
  barrel medic unknown protein, clone MTYFP_FQ_FR_FS1G-E-17, GenBank Accession No. ACJ85952.1
MNNGCEQQQYSVIETQYIRRHHKHDLRDNQCSSALVKHIKAPVHLVWSLVRRFDQPQKYKPFISRCIMQGDLSIGSVREVNVKSGLPATTSTERLEQLDDEEHILGIRIVGGDHRLRNYSSIITVHPGVIDGRPGTMVIESFVVDVPEGNTKDETCYFVEALIRYNLSSLADVSERMAVQGRTDPININP

SEQ ID NO: 50 - protein, 185 aa, Vitis vinifera
  wine grape cultivar PN40024 unnamed protein product, locus tag GSVIVT00002440001, GenBank Accession No. CAO65816.1
MSGYGCIKMEDEYIRRHHRHEIRDNQCSSSLVKHIKAPVHLVWSLVRSFDQPQKYKPFVSRCIVQGDLEIGSVREVNVKSGLPATTSTERLELLDDEEHIFGMRIVGGDHRLKNYSSIVTVHPEIIDGRPGTLVIESFVVDVPDGNTKDETCYFVEALIKCNLKSLADVSERLAIQDRTEPIDRM

SEQ ID NO: 51 - protein, 185 aa, Vitis vinifera
  wine grape cultivar PN40024 unnamed protein product, locus tag GSVIVT00006507001, GenBank Accession No. CAO69376.1
MNGNGLSSMESEYIRRHHRHEPAENQCSSALVKHIKAPVPLVWSLVRRFDQPQKYKPFISRCVVQGNLEIGSLREVDVKSGLPATTSTERLELLDDDEHILSMRIIGGDHRLRNYSSIISLHPEIIDGRPGTMVIESYVVDVPEGNTKDETCYFVEALIKCNLKSLADVSERLAVQDRTEPIDRM

SEQ ID NO: 52 - protein, 208 aa, Oryza sativa
  rice Japonica Group, cultivar Nipponbare, hypothetical protein OsJ_21703, GenBank Accession No. EAZ37364.1
MEAHVERALREGLTEEERAALEPAVMAHHTFPPSTTTATTAAATCTSLVTQRVAAPVRAVWPIVRSFGNPQRYKHFVRTCALAAGNGPSFGSVREVTVVSGPSRLPPGTERLEMLDDDRHIISFRVVGGQHRLRNYRSVTSVTEFQPPAAGPGPAPPYCVVVESYVVDVPDGNTAEDTRMFTDTVVKLNLQMLAAVAEDSSSASRRRD

SEQ ID NO: 53 - protein, 186 aa, Capsicum annuum
  pepper cultivar hanbyul, CAPIP1 protein, GenBank Accession No. AAT35532.1
MMNANGFSGVEKEYIRKHHLHQPKENQCSSFLVKHIRAPVHLVWSLVRRFDQPQKYKPFVSRCIAQGDLEIGSLREVDVKSGLPATTSTERLELLDDEEHILSFRIIGGDHRLRNYSSIISLHPEVIDGRPGTLVIESFVVDVPQGNTKDETCYFVEALINCNLKSLADVSERLAVQDRTEPIDQV

SEQ ID NO: 54 - protein, 186 aa, Populus trichocarpa
  California poplar (Western balsam poplar, black cottonwood) cultivar 383-2499 (Nisqually-1), unknown protein, clone PX0011_1113, GenBank Accession No. ABK92491.1
MNGSDAYSATEAQYVRRHHKHEPRENQCTSALVKHIKAPAHLVWSLVRRFDQPQRYKPFVSRCVMNGELGIGSVREVNVKSGLPATTSTERLELLDDEEHILGVQIVGGDHRLKNYSSIMTVHPEFIDGRPGTLVIESFIVDVPDGNTKDETCYFVEALIRCNLKSLADVSERMAVQDRVEPVNQF

SEQ ID NO: 55 - protein, 185 aa, Capsicum annuum
  pepper cultivar hanbyul, PIP1 protein, GenBank Accession No. ABF72432.1
MNANGFSGVEKEYIRKHHLHQPKENQCSSFLVKHIRAPVHLVWSLVRRFDQPQKYKPFVSRCIAQGDLEIGSLREVDVKSGLPATTSTERLELLDDEEHILSFRIIGGDHRLRNYSSIISLHPEVIDGRPGTLVIESFVVDVPQGNTKDETCYFVEALINCNLKSLADVSERLAVQDRTEPIDQV

SEQ ID NO: 56 - protein, 186 aa, Populus trichocarpa x Populus deltoides
  California poplar (Western balsam poplar, black cottonwood) x Eastern cottonwood, cultivar H11-11, unknown protein, clone WS0133_I04, GenBank Accession No. ABK96505.1
MNGSDAYSATEAQYVRRHHKHEPRENQCTSALVKHIKAPAHLVWSLVRRFDQPQRYKPFVSRCVMNGELGIGSVREVNVKSGLPATTSTERLELLDDEEHILGVQIVGGDHRLKNYSSIMTVHPEFIDGRPGTLVIESFIVDVPDGNTKDETCYFVKALIRCNLKSLADVSERMAVQDRVEPVNQF

SEQ ID NO: 57 - protein, 188 aa, Pisum sativum
  pea AT-rich element binding factor 3 (PsATF, ATF3), potential transcription factor, GenBank Accession No. AAV85853.1
MNNGGEQYSAIETQYIRRRHKHDLRDNQCSSALVKHIKAPVHLVWSLVRRFDQPQKYKPFVSRCIMQGDLGIGSVREVNVKSGLPATTSTERLEQLDDEEHILGIRIVGGDHRLRNYSSVITVHPEVIDGRPGTMVIESFVVDVPEGNTRDETCYFVEALIRGNLSSLADVSERMAVQGRTDPINVNP

SEQ ID NO: 58 - protein, 177 aa, Vitis vinifera
  wine grape cultivar PN40024 unnamed protein product, locus tag GSVIVT00027009001, GenBank Accession No. CAO39744.1
MEAQVICRHHAHEPRENQCSSVLVRHVKAPANLVWSLVRRFDQPQKYKPFVSRCVVQGDLRIGSVREVNVKTGLPATTSTERLELFDDDEHVLGIKILDGDHRLRNYSSVITVHPEIIDGRPGTLVIESFVVDVPEGNTKDDTCYFVRALINCNLKCLAEVSERMAMLGRVEPANAV

SEQ ID NO: 59 - protein, 178 aa, Vitis vinifera
  wine grape cultivar Pinot Noir hypothetical protein, clone ENTAV 115, locus tag VITISV_004915, GenBank Accession No. CAN82501.1
MMEAQVICRHHAHEPRENQCSSVLVRHVKAPANLVWSLVRRFDQPQKYKPFVSRCVVQGDLRIGSVREVNVKTGLPATTSTERLELFDDDEHVLGIKILDGDHRLRNYSSVITVHPEIIDGRPGTLVIESFVVDVPEGNTKDDTCYFVRALINCNLKCLAEVSERMAMLGRVEPANAV

SEQ ID NO: 60 - protein, 193 aa, Arachis hypogaea
  peanut pathogenesis-induced protein (PIP), GenBank Accession No. ACG76109.1
MMNGSCGGGGGGEAYGAIEAQYIRRHHRHEPRDNQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIEGRPGTMVIESFVVDVPDGNTKDETCXFVEALIRCNLSSLADVSERMAVQGRTDPINQ

SEQ ID NO: 61 - protein, 217 aa, Zea mays
  maize AT-rich element binding factor 3, clone 300908, GenBank Accession No. ACG39386.1
MVVEMDGGVGVAAGGGGGAQTPAPAPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQNYSSILTVHPEVIDGRPGTLVIESFVVDVPDGNTKDETCYFVEALLKCNLRSLAEVSEGQVIMDQTEPLDR

SEQ ID NO: 62 - protein, 217 aa, Zea mays
  maize strain B73, unknown protein, clone ZM_BFb0036A01, GenBank Accession No. ACF80077.1
MVVEMDGGVGVAAAGGGGAQTPAPPPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQNYSSILTVHPEVIDGRPGTLVIESFVVDVPDGNTKDETCYFVEALLKCNLRSLAEVSEGQVIMDQTEPLDR

SEQ ID NO: 63 - protein, 206 aa, Oryza sativa
  rice Japonica Group, cultivar Nipponbare, hypothetical protein Os06g0528300, GenBank Accession No. NP_001057772.1
MNGVGGAGGAAAGKLPMVSHRRVQWRLADERCELREEEMEYIRRFHRHEPSSNQCTSFAAKHIKAPLHTVWSLVRRFDQPQLFKPFVRNCVMRENIIATGCIREVNVQSGLPATRSTERLELLDDNEHILKVNFIGGDHMLKNYSSILTVHSEVIDGQLGTLVVESFIVDVPEGNTKDDISYFIENVLRCNLRTLADVSEERLANP

SEQ ID NO: 64 - protein, 206 aa, Oryza sativa
  rice Indica Group, cultivar 93-11, hypothetical protein OsI_23215, GenBank Accession No. EAZ01188.1
MNGAGGAGGAAAGKLPMVSHRQVQWRLADERCELREEEMEYIRQFHRHEPSSNQCTSFVAKHIKAPLQTVWSLVRRFDQPQLFKPFVRKCVMRENIIATGCVREVNVQSGLPATRSTERLELLDDNEHILKVKFIGGDHMLKNYSSILTIHSEVIDGQLGTLVVESFVVDIPEGNTKDDICYFIENILRCNLMTLADVSEERLANP

SEQ ID NO: 65 - protein, 205 aa, Oryza sativa
  rice Japonica Group, cultivar Nipponbare, hypothetical protein OsJ_06125, GenBank Accession No. EAZ22456.1
MVEVGGGAAEAAAGRRWRLADERCDLRAAETEYVRRFHRHEPRDHQCSSAVAKHIKAPVHLVWSLVRRFDQPQLFKPFVSRCEMKGNIEIGSVREVNVKSGLPATRSTERLELLDDNEHILSVRFVGGDHRLKNYSSILTVHPEVIDGRPGTLVIESFVVDVPEGNTKDETCYFVEALLKCNLKSLAEVSERLVCQGPNRAPSTR

SEQ ID NO: 66 - protein, 204 aa, Oryza sativa
  rice Japonica Group, cultivar Nipponbare, hypothetical protein Os02g0255500, similar to extensin (fragment), GenBank Accession No. NP_001046464.1
MVEVGGGAAEAAAGRRWRLADERCDLRAAETEYVRRFHRHEPRDHQCSSAVAKHIKAPVHLVWSLVRRFDQPQLFKPFVSRCEMKGNIEIGSVREVNVKSGLPATRSTERLELLDDNEHILSVRFVGGDHRLKNYSSILTVHPEVIDGRPGTLVIESFVVDVPEGNTKDETCYFVEALLKCNLKSLAEVSERLVVKDQTEPLDR

SEQ ID NO: 67 - protein, 199 aa, Medicago truncatula
  barrel medic unknown protein, clone MTYFP_FQ_FR_FS1G-G-11, GenBank Accession No. ACJ86004.1
MEKMNGTENNGVFNSTEMEYIRRHHNQQPGENQCSSALVKHIRAPVPLVWSLVRRFDQPQKYKPFVSRCVVRGNLEIGSLREVDVKSGLPATTSTERLEVLDDNEHILSIRIIGGDHRLRNYSSIMSLHPEIIDGRPGTLVIESFVVDVPEGNTKDETCYFVEALIKCNLKSLSDVSEGHAVQDLTEPLDRVHELLISG

SEQ ID NO: 68 - protein, 199 aa, Medicago truncatula
  barrel medic unknown protein, clone MTYF1_F2_F3_FY1G-K-4, GenBank Accession No. ACJ83958.1
MEKMNGTENNGVFNSTEMEYIRRHHNQQPGENQCSSALVKHIRAPVPLVWSLVRRFDQPQKYKPFVSRCVVRGNLEIGSLREVDVKSGLPATTSTERLEVLDDNEHILSIRIIGGDHRLRNYSSIMSLHPEIIDGRPGTLVIESFVVDVPEGNTKDETCYFVEALIKCNLKSLSDVSEGHAAQDLTEPLDRMHELLISG

SEQ ID NO: 69 - protein, 197 aa, Zea mays
  maize CAPIP1 protein, clone 244179, GenBank Accession No. ACG34726.1
MVGLVGGSTARAEHVVANAGGEAEYVRRMHRHAPTEHQCTSTLVKHIKAPVHLVWQLVRRFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLNSLAEVSEQLAVESPTSLIDQ

SEQ ID NO: 70 - protein, 197 aa, Zea mays
  maize CAPIP1 protein, clone 1448906, GenBank Accession No. ACG26022.1
MVGLVGGSTARAEHVVANAGGEAEYVRRMHRHAPTEHQCTSTLVKHIKAPVHLVWELVRRFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLNSLAEVSEQLAVESPTSLIDQ

SEQ ID NO: 71 - protein, 212 aa, Zea mays
  maize strain B73 unknown protein, clone ZM_BFc0183D21, GenBank Accession No. ACF86162.1
MVMVEMDGGVGGGGGGGQTPAPRRWRLADERCDLRAMETDYVRRFHRHEPREHQCSSAVAKHIKAPVHLVWSLVRRFDQPQLFKPFVSRCEMKGNIEIGSVREVNVKSGLPATRSTERLELLDDNEHILSVRFVGGDHRLQNYSSILTVHPEVIDGRPGTLVIESFVVDVPDGNTKDETCYFVEALLKCNLKSLAEVSERQVVKDQTEPLDR

SEQ ID NO: 72 - protein, 205 aa, Oryza sativa
  rice Japonica Group, cultivar Nipponbare, conserved hypothetical protein Os06g0527800, GenBank Accession No. NP_001057771.1
MNGAGGAGGAAAGKLPMVSHRRVQCRLADKRCELREEEMEYIRQFHRHEPSSNQCTSFVAKHIKAPLQTVWSLVRRFDQPQLFKPFVRKCVMRENIIVTGCVREVNVQSGLPATRSTERLELLDDNEHILKVKFIGGDHMLKNYSSILTIHSEVIDGQLGTLVVESFVVDIPDGNTKDDICYFIENVLRCNLMTLADVSEERLAN

SEQ ID NO: 73 - protein, 197 aa, Zea mays
  maize strain B73 unknown protein, clone ZM_BFc0063E17, GenBank Accession No. ACF85073.1
MVGLVGGSTARAEHVVANAGGETEYVRRLHRHAPAEHQCTSTLVKHIKAPVHLVWELVRSFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLKSLAEVSEQLAVESPTSPIDQ

SEQ ID NO: 74 - protein, 206 aa, Oryza sativa
  rice Indica Group, cultivar 93-11, hypothetical protein OsI_23218, GenBank Accession No. EAZ01191.1
MNGVGGAGGAAAGKLPMVSHRRVQWRLADERCELREEEMEYIRRFHRHEPSSNQCTSFAAKHIKAPLHTVWSLVRRFDQPQLFKPFVRNCVMRENIIATGCIREVNVQSGLPATRSTERLELLDDNEHILKVKFIGGDHMLKNYSSILTVHSEVIDGQLGTLVVESFIVDVLEGNTKDDISYFIENVLRCNLRTLADVSEERLANP

SEQ ID NO: 75 - protein, 209 aa, Oryza sativa
  rice Japonica Group, cultivar Nipponbare, conserved hypothetical protein Os05g0213500, GenBank Accession No. NP_001054923.1
MVGLVGGGGWRVGDDAAGGGGGGAVAAGAAAAAEAEHMRRLHSHAPGEHQCSSALVKHIKAPVHLVWSLVRSFDQPQRYKPFVSRCVVRGGDLEIGSVREVNVKTGLPATTSTERLELLDDDEHILSVKFVGGDHRLRNYSSIVTVHPESIDGRPGTLVIESFVVDVPDGNTKDETCYFVEAVIKCNLTSLAEVSERLAVQSPTSPLEQ

SEQ ID NO: 76 - protein, 180 aa, Oryza sativa
  rice Japonica Group, cultivar Nipponbare, Bet v I allergen-like protein, clone OSJNBa0052K15, gene OSJNBa0052K15.17, GenBank Accession No. BAD29692.1
MVEMDAGGRPEPSPPSGQCSSAVTMRINAPVHLVWSIVRRFEEPHIFQPFVRGCTMRGSTSLAVGCVREVDFKSGFPAKSSVERLEILDDKEHVFGVRIIGGDHRLKNYSSVLTAKPEVIDGEPATLVSESFVVDVPEGNTADETRHFVEFLIRCNLRSLAMVSQRLLLAQGDLAEPPAQ

SEQ ID NO: 77 - protein, 176 aa, Vitis vinifera
  wine grape cultivar Pinot Noir hypothetical protein, clone ENTAV 115, locus tag VITISV_029498, GenBank Accession No. CAN64668.1
MNGNGLSSMESEYIRRHHRHEPAENQCSSALVKHIKAPVPLVWSLVRRFDQPQKYKPFISRCVVQGNLEIGSLREVDVKSGLPATTSTERLELLDDDEHILSMRIIGGDHRLRNYSSIISLHPEIIDGRPGTMVIESYVVDVPEGNTKDETCYFSLADVSERLAVAGTVTEPIDRM

SEQ ID NO: 78 - protein, 180 aa, Oryza sativa
  rice Indica Group, cultivar 93-11, hypothetical protein, locus tag OsI_06615, GenBank Accession No. EEC72859.1
MVEMDAGGRPEPSPPSGQCSSAVTMRINAPVHLVWSIVRRFEEPHIFQPFVRGCTMRGSTSLAVGCVREVDFKSGFSAKSSVERLEILDDKEHVFGVRIIGGDHRLKNYSSVLTAKPEVIDGEPATLVSESFVIDVPEGNTADETRHFVEFLIRCNLRSLAMVSQRLLLAQGDLAEPPAQ

SEQ ID NO: 79 - protein, 215 aa, Oryza sativa
  rice Japonica Group, cultivar Nipponbare, hypothetical protein, locus tag OsJ_10498, GenBank Accession No. EAZ26598.1
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGLGKVRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 80 - protein, 186 aa, Rheum australe
  Himalayan rhubarb pathogen-induced protein-like protein, GenBank Accession No. ACH63237.1
MNGDGYGGSEEEFVKRYHEHVLADHQCSSVLVEHINAPLHLVWSLVRSFDQPQKYKPFVSRCVVQGGDLEIGSVREVDVKSGLPATTSMEELELLDDKEHVLRVKFVGGDHRLKNYSSIVSLHPEIIGGRSGTMVIESFIVDIADGNTKEETCYFIESLINCNLKSLSCVSERLAVEDIAERIAQM

SEQ ID NO: 81 - protein, 254 aa, Oryza sativa
  rice Japonica Group, cultivar Nipponbare, hypothetical protein, locus tag OsJ_016770, GenBank Accession No. EAZ33287.1
MVGLVGGGGWRVGDDAAGGGGGGAVAAGAAAAAEAEHMRRLHSQGPRRAPVQLRARQAHQGSCSPPRIECANFAVFLAARDPKIVWSLVRSFDQPQRYKPFVSRCVVRGGDLEIGSVREVNVKTGLPATTSTERLELLDDDEHILSVKFVGGDHRLRNYSSIVTVHPESIDGRPGTLVIESFVVDVPDGNTKDETCYFVEAVIKCNLTSLAEMVRMISLVLPFMLVDRMSGITCESHLETTLVRCGEYAVLAHV

SEQ ID NO: 82 - protein, 186 aa, Oryza sativa
  rice Japonica Group, cultivar Nipponbare, hypothetical protein, locus tag OsJ_005784, GenBank Accession No. EAZ22301.1
MEPHMERALREAVASEAERRELEGVVRAHHTGWNAPLAAVWPHRARVRPTRSGTSTSSSRASSPPGDGATVGSVREVAVVSGLPASTSTERLEILDDDRHVLSFRVVGGDHRLRNYRSVTSVTEFSSPSSPPRPYCVVVESYVVDVPEGNTEEDTRMFTDTVVKLNLQKLAAVATSSSPPAAGNHH

SEQ ID NO: 83 - protein, 150 aa, Oryza sativa
  rice Japonica Group, cultivar Nipponbare, hypothetical protein, locus tag OsJ_005938, GenBank Accession No. EAZ22455.1
MEVVWSIVRRFEEPHIFQPFVRGCTMRGSTSLAVGCVREVDFKSGFPAKSSVERLEILDDKEHVFGVRIIGGDHRLKNYSSVLTAKPEVIDGEPATLVSESFVVDVPEGNTADETRHFVEFLIRCNLRSLAMVSQRLLLAQGDLAEPPGQ

SEQ ID NO: 84 - protein, 206 aa, Oryza sativa
  rice Japonica Group, cultivar Nipponbare, hypothetical protein, locus tag OsJ_018129, GenBank Accession No. EAZ34646.1
MPYTAPRPSPPQHSRIGGCGGGGVLKAAGAAGHAASCVAVPAEVARHHEHAAGVGQCCSAVVQAIAAPVDAVWRTSTSSGAAASWTATATAGPLPVGSVREFRVLSGLPGTSSRERLEILDDERRVLSFRVVGGEHRLSNYRSVTTVHETAAGAAAAVVVESYVVDVPHGNTADETRMFVDTIVRCNLQSLARTAEQLALAAPRAA

SEQ ID NO: 85 - protein, 396 aa, Vitis vinifera
  wine grape cultivar Pinot Noir hypothetical protein, clone ENTAV 115, locus tag VITISV_001710, GenBank Accession No. CAN76441.1
MPISSLPFSLYTVTPNPLKLITTHAHAFTPHTHIFTLKFMSHTYCPHIHHITSIHYTHLLXPIPHMPLQPPLPPHPILPSMPAFQHLYSTNQHLQVALFSARGPNIRDFNFQDADLLKLDILAPGSLIWAAWSPNGTDEANYVGEGSPTVAMIAKRGPRHGKYMAFCXMYRDNVAPKGVNXAVATVKTKRTIQLKTSLEIACHYAGINISGINGEVMPGQWEYQVGPGQCSSLLAQRVHVPLSAVGSVVHRFDKPQRYQHVIKSCRIEDGFEMRMGXLRDVNIISGLPTATNTGRLDMQDDERHVTRCPHQRQSESKYTENNNSDASSIKSPINGPSEHLKTAASPKTESIIVIDTSKFLNEEDFEGKDETSSSNQVQIEDENWETRFPNTDAGIW

SEQ ID NO: 86 - protein, 443 aa, Vitis vinifera
  wine grape cultivar Pinot Noir hypothetical protein, clone ENTAV 115, locus tag VITISV_014403, GenBank Accession No. CAN9881.1
MPSAXKSSTVPLSLXQFKLGLRHGHRVIPWGDLDSLAMLQRQLDVDILVTGHTHRFTAYKHEGGVVINPGSATGAFGSITYDVNPSFVLMDIDGLRVVVCVYELIDETANIIKELHARKISFGTKSMIXCLLLKRRSTPKFRRKKLFLFQCRVQMTLTLTNLAVSGIAQTLQVDQWTVCALIFMTRRDIHLDKARFLDFKDMGKLLADASGLRKALSGGXVTAGMAIFDTMRHIRPDVPTVCVGLAAVAMIAKRGPRHGKYMAFCPMYRDNVAPKGVNVAVVTVKTKRTIQLKTSLEIACHYAGINISGINGEVMPGQWEYQVGPGQCSSLLAQRVHVPLSAVGSVVHRFDKPQRYQHVIKSCRIEDGFEMRMGRLRDVNIISGLPTATNTGRLDMQDDEXHVTRCPHQRQSESKYTENNNSDASSVKSPINGPSEHLKTAAX

SEQ ID NO: 87 - protein, 95 aa, Oryza sativa
  rice Indica Group, cultivar Pokkali, capip1 protein, clone OSR-385-428-D5, GenBank Accession No. ABR25904.1
EIGSVREVNVKTGLPATTSTERLELLDDDEHILSVKFVGGDHRLRNYSSIVTVHPESIDGRPGTLVIESFVVDVPDGNTKDETCYFVEAVIKCNL

SEQ ID NO: 88 - protein, 191 aa, Zea mays
  maize strain B73 unknown protein, clone ZM_BFc0034O07, GenBank Accession No. ACF84624.1
MVVEMDGGVGVAAAGGGGAQTPAPPPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQVCSVLHLSIFCAAHARYFAHHLKCVLEFLCQMHLDVLPCDDAILE

SEQ ID NO: 89 - protein, 239 aa, Oryza sativa
  rice Japonica Group, cultivar Nipponbare, hypothetical protein, locus tag OsJ_020681, GenBank Accession No. EAZ37198.1
MNGCTGGAGGVAAGRLPAVSLQQAQWKLVDERCELREEEMEYVRRFHRHEIGSNQCNSFIAKHVRAPLQNVWSLVRRFDQPQIYKPFVRKCVMRGNVETGSVREIIVQSGLPATRSIERLEFLDDNEYILRVKFIGGDHMLKKRIPKKTYAISSRTCSDSAIIAVGQSNCAPEITAMNGGVSIQPWLILLAFFSSPSNQTNPDSLRDMHPGSWFQILLVLAMFTCSKGSVLPPSEKVNV

SEQ ID NO: 90 - protein, 188 aa, Zea mays
  maize GRMZM2G154987_P01 protein
MEPHMESALRQGLSEAEQRELEGVVRAHHTFPGRAPGTCTSLVTQRVDAPLAAVWPIVRGFGSPQRYKHFIKSCDLKAGDGATVGSVREVTVVSGLPASTSTERLEILDDHRHILSFRVVGGDHRLRNYRSVTSVTEFQPGPYCVVLESYVVDVPDGNTEEDTRMFTDTVVKLNLQKLAAIATSSSAN

SEQ ID NO: 91 - protein, 205 aa, Zea mays
  maize GRMZM2G134731_P01 protein
MDQQGAGGDVEVPAGLGLTAAEYEQLRPTVDAHHRYAVGEGQCSSLLAQRIHAPPAAVWAIVRRFDCPQVYKHFIRSCAVRPDPDAGDALRPGRLREVCVISGLPASTSTERLDHLDDAARVFGFSITGGEHRLRNYRSVTTVSELAGPGICTVVLESYAVDVPDGNTEDDTRLFADTVIRLNLQKLKSVAEASTSSSAPPPPSE

SEQ ID NO: 92 - protein, 220 aa, Zea mays
  maize GRMZM2G144224_P01 protein
MPCIQASSPGGMPHQHGRGRVLGGGVGCAAEVAAAVAASAGGMRCGAHDGEVPAEAARHHEHAAAGPGRCCSAVVQHVAAPAAAVWSVVRRFDQPQVYKRFVRSCALLAGDGGVGTLREVRVVSGLPAASSRERLEVLDDESHVLSFRVVGGEHRLRNYLSVTTVHPSPAAPDAATVVVESYVVDVPPGNTPEDTRVFVDTIVKCNLQSLATTAEKLAAV

SEQ ID NO: 93 - protein, 221 aa, Glycine max
  soybean Glyma01g02290.1 protein
MEKAESSASTSEPDSDENHHRHPTNHHINPPSGLTPLEFASLIPSVAEHHSYLVGSGQCSSLLAQRVQAPPDAVWSVVRRFDKPQTYKHFIKSCAVKEPFHMAVGVTRDVNVISGLPAATSTERLDLLDDIRCVTGFSIIGGEHRLRNYRSVTTVHSFEDDADDGKIYTVVLESYVVDVPDGNTEEDTRLFADTVVKLNLQKLASVTEGTNRDGDGKSHSR

SEQ ID NO: 94 - protein, 214 aa, Glycine max
  soybean Glyma01g12970.1 protein
MEKTHSSSAEEQDPTRRHLDPPPGLTAEEFEDLKPSVLEHHTYSVTPTRQSSSLLAQRIHAPPHAVWSVVRCFDNPQAYKHFIKSCHVKEGFQLAVGSTRDVHVISGLPAATSTERLDLLDDDRHVIGFTIVGGDHRLRNYRSVTSVHGFECDGKIWTVVLESYVVDVPEGNTEEDTRLFADTVVKLNLQKLASVSEGMCGDGDGDGDGKGNKS

SEQ ID NO: 95 - protein, 216 aa, Glycine max
  soybean Glyma01g31320.1 protein
MLQNSSMSSLLLHRINGGGGATTATNCHDTVFMTVPDGVARYHTHAVAPNQCCSSVAQEIGASVATVWSVLRRFDNPQAYKHFVKSCHVIGGDGDVGTLREVHVISGLPAARSTERLEILDDERHVISFSVVGGDHRLANYRSVTTLHPTASSASGGCSGTVVVESYVVDVPPGNTREDTRVFVDTIVKCNLQSLAQTAENLTLRKNNNNDYKCCS

SEQ ID NO: 96 - protein, 208 aa, Glycine max
  soybean Glyma02g42990.1 protein
MTSLQFHRFNPATDTSTAIANGVNCPKPPSTLRLLAKVSLSVPETVARHHAHPVGPNQCCSVVIQAIDAPVSAVWPVVRRFDNPQAYKHFVKSCHVVAAAGGGEDGIRVGALREVRVVSGLPAVSSTERLEILDDERHVMSFSVVGGDHRLRNYRSVTTLHGDGNGGTVVIESYVVDVPPGNTKEETCVFVDTIVRCNLQSLAQIAET

SEQ ID NO: 97 - protein, 176 aa, Glycine max
  soybean Glyma04g05380.1 protein
AYPVLGLTPEEFSELESIINTHHKFEPSPEICSSIIAQRIDAPAHTVWPLVRSFENPQKYKHFVKSCNMRSGDGGVGSIREVTVVSGLPASTSTERLEILDDDKHLLSFRVVGGEHRLHNYRSVTSVNEFKNPDNGKVYTIVLESYVVDIPEGNTGVDTKMFVDTVVKLNLQKLGE

SEQ ID NO: 98 - protein, 172 aa, Glycine max
  soybean Glyma06g05440.1 protein
EFTELESTINTHHKFEASPEICSSIIAQRIDAPAHTVWPLVRSFENPQKYKHFVKSCNMRSGDGGVGSIREVTVVSGLPASTSTERLEILDDDNHLLSFRVVGGEHRLHNYRSVTSVNEFKRPDNGKVYTIVLESYVVDIPEGNTGVDTKMFVDTVVKLNLQKLGEVAMATN

SEQ ID NO: 99 - protein, 191 aa, Glycine max
  soybean Glyma06g13150.1 protein
MTELSSREVEYIRRHHSKAAEDNQCASALVKHIRAPLPLVWSLVRRFDEPQKYKPFVSRCVVRGNLEIGSLREVDVKSGLPATTSTERLEILDDNHHILSVRIIGGDHRLRNYSSIMSLHPEIVDGRPGTLVIESFVVDIPEGNTKDETCYFVEALIKCNLKSLADVSEGLTLQDHTEPIDRKYELLITRG

SEQ ID NO: 100 - protein, 185 aa, Glycine max
  soybean Glyma07g06270.1 protein
MNGGESYGAIETQYIRRHHKHEPRENQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIDGRPGTMVIESFVVDVPDGNTRDETCYFVEALIRCNLSSLADVSERMAVQGRTNPINH

SEQ ID NO: 101 - protein, 178 aa, Glycine max
  soybean Glyma07g19120.1 protein
MSPNNPSTIVSDAVARHHTHVVSPHQCCSAVVQEIAAPVSTVWSVVRRFDNPQAYKHFVKSCHVILGDGDVGTLREVRVISGLPAAVSTERLDVLDDERHVIGFSMVGGDHRLSNYRSVTILHPRSATDTVVVESYVVDVPAGNTTEDTRVFVDTILRCNLQSLAKFAENLTNKLHQR

SEQ ID NO: 102 - protein, 246 aa, Glycine max
  soybean Glyma08g36770.1 protein
MSRSHNKRKPFSFIFKITLLELLSSLLSSSLRFAMDKTHSGEEQDPNPTHPTRNHLDPPPGLTPEEFEDLKPSVLEHHTYSVTPTRQCSSLLAQRIHAPPHTVWTVVRCFDNPQAYKHFIKSCHVKEGFQLAVGSTRDVHVISGLPAATSTERLDLLDDDRHVIGFTIVGGDHRLRNYRSVTSVHGFERDGKIWTVVLESYVVDVPEGNTEEDTRLFADTVVKLNLQKLASVTEGMCGDSDGKGNN

SEQ ID NO: 103 - protein, 223 aa, Glycine max
  soybean Glyma09g33700.1 protein
MEKAESSASTSEPDSDDNHHRHPTNHHLNPPSGLTPLEFASLVPSVAEHHSYLVGPGQCSSLLAQRVHAPPDAVWSFVRRFDKPQTYKHFIKSCAVKEPFHMAVGVTRDVNVISGLPAATSTERLDFLDDVRRVTGFSIIGGEHRLRNYRSVTTVHSFDDDNASADGKIYTVVLESYVVDVPDGNTEEDTRLFADTVVKLNLQKLASVTEGTNGDGDGKPHSR

SEQ ID NO: 104 - protein, 229 aa, Glycine max
  soybean Glyma11g35670.1 protein
MPSSLHFDRFNPITHAATTVAIANGVNCPKQPQAPPSSTAARRLVVPSLSSGRGIAAPDTVALHHAHVVDPNQCCSIVTQHINAPVSAVWAVVRRFDNPQGYKNFVRSCHVITGDGIRVGAVREVRVVSGLPAETSTERLEILDDERHVISFSMVGGDHRLRNYQSVTTLHANGNGTLVIESYVVDVPQGNTKEETCVFVDTIVRCNLQSLAQIAENRTNNCEHTAQHC

SEQ ID NO: 105 - protein, 191 aa, Glycine max
  soybean Glyma13g08120.1 protein
MNGIGNDGGGGLSNVEMEYIRRHHRHEPGENQCGSALVKHIRAPVPQVWSLVRRFDQPQKYKPFVSRCVVRGNLEIGSLREVDVKSGLPATTSTERLELLDDNEHLLSIRIIGGDHRLRNYSSIMSLHPEIIDGRPGTLVIESFVVDVPEGNTKDETCYFVEALIKCNLKSLADVSEGIAVQDRTEPIDRI

SEQ ID NO: 106 - protein, 169 aa, Glycine max
  soybean Glyma14g06100.1 protein
MVARHHAHAVGPNQCCSFVIQAIDAPVSAVWPVVRRFDNPQAYKHFVKSCHVVAAGGAGGDGGIHVGALREVRVVSGLPAVSSTERLEILDDERHVMSFSVVGGDHRLRNYRSVTTLHGDGSNGGTVVIESYVVDIPAGNTKEETCVFVDTIVRCNLQSLAQMAENMGS

SEQ ID NO: 107 - protein, 210 aa, Glycine max
  soybean Glyma14g10730.1 protein
MTILPHSNNKSSNHKFIAHQNYMASETHHHVQGLTPEELTKLEPIIKKYHLFEQSPNTCFSIITYRIEAPAKAVWPFVRSFDNPQKYKHFIKGCNMRGDGGVGSIREVTVVSGLPASTSTERLEILDDDKHVLSFRVVGGEHRLKNYRSVTSVNEFNKEGKVYTIVLESYIVDIPEGNTEEDTKMFVDTVVKLNLQKLGVVAMASSMHGQ

SEQ ID NO: 108 - protein, 193 aa, Glycine max
  soybean Glyma14g30260.1 protein
MNRIGNGGGGGGGLSNVEMEYIRRHHRHEPGENQCGSALVKHIRAPVPQVWSLVRRFDQPQKYKPFISRCVVRGNLEIGSLREVDVKSGLPATTSTERLELLDDNEHILSIRIIGGDHRLRNYSSIMSLHPEIIDGRPGTLVIESFVVDVPEGNTKDETCYFVEALIKCNLKSLADVSEGLAVQDCTEPIDRI

SEQ ID NO: 109 - protein, 188 aa, Glycine max
  soybean Glyma17g34800.1 protein
MASETHHHVQGLTPEELTQLEPIIKKYHLFEASSNKCFSIITHRIEAPASSVWPLVRNFDNPQKYKHFIKGCNMKGDGSVGSIREVTVVSGLPASTSTERLEILDDDKHVLSFRVVGGEHRLQNYRSVTSVNEFHKEGKVYTIVLESYIVDIPEGNTEEDTKMFVDTVVKLNLQKLGVVAMASSMNGR

SEQ ID NO: 110 - protein, 177 aa, Glycine max
  soybean Glyma18g43680.1 protein
MLPNNPSTIVPDAVARHHTHVVSPQQCCSAVVQEIAAPVSTVWSVVRRFDNPQAYKHFVKSCHVILGDGDVGTLREVHVISGLPAAVSTERLDVLDDERHVIGFSMVGGDHRLFNYRSVTTLHPRSAAGTVVVESYVVDVPPGNTTEDTRVFVDTILRCNLQSLAKFAENLTKLHQR

SEQ ID NO: 111 - protein, 185 aa, Glycine max; identical to SEQ ID NO: 100
  soybean Glyma07g06270.2 protein

SEQ ID NO: 112 - protein, 191 aa, Glycine max
  soybean Glyma16g02910.1 protein
MGITIGIQCLEIEEISICDGMFCYLVDFVDVKEKMNYCLMWFGYFPSQVWSLVRRFDQPQKYKPFVSRCIMQGDLGIGSVREVNVKSGLPATTSTERLEQLDDEEHILGIRIVGGDHRLRNYSSIITVHPEVIDGRPSTMVIESFVVDVPDGNTRDETCYFVEALIRCNLSSLADVSERMAVQGRTDPINH

SEQ ID NO: 113 - protein, 185 aa, Artificial Sequence
  synthetic PYR/PYL receptor protein
MNGGESYGAIETQYIRRHHKHEPRENQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIDGRPSTMVIESFVVDVPDGNTRDETCYFVEALIRCNLSSLADVSERMAVQGRTDPINH

SEQ ID NO: 114 - protein, 204 aa, Sorghum bicolor
  sorghum Sb10g022200 protein
METHVERALRATLTEAEVRALEPAVREHHTFPAGRVAAGTTTPTPTTCTSLVAQRVSAPVRAVWPIVRSFGNPQRYKHFVRTCALAAGDGASVGSVREVTVVSGLPASSSTERLEVLDDDRHILSFRVVGGDHRLRNYRSVTSVTEFQPGPYCVVVESYAVDVPEGNTAEDTRMFTDTVVRLNLQKLAAVAEESAAAAAAGNRR

SEQ ID NO: 115 - protein, 204 aa, Sorghum bicolor
  sorghum Sb04g008040 protein
MEPHMETALRQGGLSELEQRELEPVVRAHHTFPGRSPGTTCTSLVTQRVDAPLSAVWPIVRGFAAPQRYKHFIKSCDLRSGDGATVGSVREVTVVSGLPASTSTERLEILDDDRHILSFRVVGGDHRLRNYRSVTSVTEFHHHHQAAAGRPYCVVVESYVVDVPEGNTEEDTRMFTDTVVKLNLQKLAAIATSSAAAAASNSST

SEQ ID NO: 116 - protein, 258 aa, Sorghum bicolor
  sorghum Sb01g028330 protein
MVESPNPNSPSRPLCIKYTRAPARHFSPPLPFSSLIISANPIEPKAMDKQGAGGDVEVPAGLGLTAAEYEQLRSTVDAHHRYAVGEGQCSSLLAQRIQAPPAAVWAIVRRFDCPQVYKHFIRSCALRPDPEAGDALRPGRLREVSVISGLPASTSTERLDLLDDAARVFGFSITGGEHRLRNYRSVTTVSELADPGICTVVLESYVVDVPDGNTEDDTRLFADTVIRLNLQKLKSVAEANAAAAASFVSVVPPPEPEE

SEQ ID NO: 117 - protein, 222 aa, Sorghum bicolor
  sorghum Sb01g038150 protein
MPCLQASSSPGSMPHQHHGRVLAGVGCAAEVAAAAVAATSPAAGMRCGAHDGEVPAEAARHHEHAAPGPGRCCSAVVQHVAAPASAVWSVVRRFDQPQAYKRFVRSCALLAGDGGVGTLREVRVVSGLPAASSRERLEVLDDESHVLSFRVVGGEHRLQNYLSVTTVHPSPAAPDAATVVVESYVVDVPPGNTPEDTRVFVDTIVKCNLQSLATTAEKLAAV

SEQ ID NO: 118 - protein, 211 aa, Sorghum bicolor
  sorghum Sb04g009280 protein
MVEMDGGVGVVGGGQQTPAPRRWRLADELRCDLRAMETDYVRRFHRHEPRDHQCSSAVAKHIKAPVHLVWSLVRRFDQPQLFKPFVSRCEMKGNIEIGSVREVNVKSGLPATRSTERLELLDDNEHILSVKFVGGDHRLQNYSSILTVHPEVIDGRPGTLVIESFVVDVPDGNTKDETCYFVEALLKCNLKSLAEVSERQVIKDQTEPLDR

SEQ ID NO: 119 - protein, 216 aa, Sorghum bicolor
  sorghum Sb09g023180 protein
MPYTAPRPSPQQHSRVTGGGAKAAIVAASHGASCAAVPAEVARHHEHAARAGQCCSAVVQAIAAPVGAVWSVVRRFDRPQAYKHFIRSCRLVDDGGGGAGAGAGATVAVGSVREVRVVSGLPATSSRERLEILDDERRVLSFRVVGGEHRLANYRSVTTVHEAEAGAGGTVVVESYVVDVPPGNTADETRVFVDTIVRCNLQSLARTAERLALALA

SEQ ID NO: 120 - protein, 36 aa, Artificial Sequence
  synthetic consensus sequence, amino acid residues 30-65 of PYR1
CXSXXXXXXXAPXXXXWXXXXXFXXPXXXXXFXXXC

SEQ ID NO: 121 - protein, 25 aa, Artificial Sequence
  synthetic consensus sequence, amino acid residues 76-100 of PYR1
GXXRXVXXXSXXPAXXSXEXLXXXD

SEQ ID NO: 122 - protein, 11 aa, Artificial Sequence
  synthetic consensus sequence, amino acid residues 112-122 of PYR1
GGXHRLXNYXS

SEQ ID NO: 123 - protein, 31 aa, Artificial Sequence
  synthetic consensus sequence, amino acid residues 141-171 of PYR1
ESXXVDXPXGXXXXXTXXFXXXXXXXNLXXL

SEQ ID NO: 124 - protein, 191 aa, Artificial Sequence
  synthetic fenhexamid-responsive PYR/PYL receptor mutant clone #27-18
MPSELTPEERSELKNSIAEFHTYQLDPGSCSSLHAQRIHAPPELVWPIVRRFDKPQTYRHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRMFADTVVKLNLQKLATVAEAMARNSGDGSGSQVT

SEQ ID NO: 125 - protein, 191 aa, Artificial Sequence
  synthetic fenhexamid-responsive PYR/PYL receptor mutant clone #27-24
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNHKSVTTVHRFEKENRIWTVVLESYAVDMPEGNSEDDTRMFADTVVKLNLQKLATVAEAMARNSGDGSGSQVT

SEQ ID NO: 126 - protein, 191 aa, Artificial Sequence
  synthetic fenhexamid-responsive PYR/PYL receptor mutant clone #27-31
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLDILDDERRVTGFSIIGGEHRLTNCKSVTTVHRFEKENRIWTVVLESYVVDMPEGNSEDDTRMFADTVVKLNLQKLATVAEAMARNSGDGSGSQVT

SEQ ID NO: 127 - protein, 191 aa, Artificial Sequence
  synthetic fenhexamid-responsive PYR/PYL receptor mutant clone #27-28
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLDILDDERRVTGFSIIGGEHRLTNCKSVMTVHRFEKENRIWTVVLESYVVDMPEGNSEDDTRMFADTVVKLNLQKLATVAEAMARNSGDGSGSQVT

SEQ ID NO: 128 - protein, 191 aa, Artificial Sequence
  synthetic fenhexamid-responsive PYR/PYL receptor mutant clone #27-9
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLDILDDERRVTGFSIIGGEHRLTNCKSVTTVHRFEKENRIWTVVLESYVVDMPEGNSGDDTRMFADTVVKLNLQKLATVAEAMARNSGDGSGSQVT

SEQ ID NO: 129 - protein, 191 aa, Artificial Sequence
  synthetic fenhexamid-responsive PYR/PYL receptor mutant clone #27-36
MPSELTPEERSGLKNSIAEFHTYQLDPGSCSSLHAQRIHAPPGLVWSIVRRFDKPQTYRHFIKSCSVEQNFEMRVGCTRDVIVISGLPANTSTERLDILDDERRVTGFSSIGGEHRLTNYKSVTTVHRFEKEDRIWTVVLESYVVDMPEGNSEDDTRMFADTVVKLNLQKLATVAEAMARNSGDGSGSQVT

SEQ ID NO: 130 - protein, 191 aa, Artificial Sequence
  synthetic fenhexamid-responsive PYR/PYL receptor mutant clone #27-14
MPSELTPEERSGLKNSIAEFHTYQRDPGSCSSLHAQRIHAPPGLVWSIVRRFDKPQTYRHFIKSCSVEQNFEMRVGCTRDVIVISGLPANTSTERLDILDDERRVTGFSSIGGEHRLTNYKSVTTVHRFEKEDRIWTVVLESYVVDMPEGNSEDDTRMFADTVVKLNLQKLATVAEAMARNSGDGSGSQVT

SEQ ID NO: 131 - protein, 191 aa, Artificial Sequence
  synthetic bromoxynil-responsive PYR/PYL receptor mutant clone #74A-12
MPSELTPEERSELKNSIAEFHTYQLDPGSCSSLHAQRIHAPPELVWSIVRRFDKPQAYRHFIKSCSVEQNFEMRVGCTRDVIVISGLPANTSTERLDILDDERRVTGFSIIGGEHRLTNYKSVTTVHRFEKENRIWTVVLGSYVVDMPEGNSEDDTRMFADTVVKLNLQKLATVAEAMARNSGDGSGSQVT

SEQ ID NO: 132 - protein, 191 aa, Artificial Sequence
  synthetic bromoxynil-responsive PYR/PYL receptor mutant clone #74A-24
MPSELTPEERSELKNSIAEFHTYQLDPGSCSSLHAQRIHAPPELVWSIVGRFDKPQTYRHFIKSCSVEQNFEMRVGCTRDVIVISGLPANTSTERLDILDDERRVTGFSIIGGEHRLTNYKSVTTVHRFEKENRIWTVVLQSYVVDMPEGNSEDDTRMFADTVVKLNLQKLATVAEAMARNSGDGSGSQVT

SEQ ID NO: 133 - protein, 191 aa, Artificial Sequence
  synthetic bromoxynil-responsive PYR/PYL receptor mutant clone #74A-1
MPSELTPEERSELKNSIAEFHTYQLDPGSCSSLHAQRIHAPPELVWSIVRRFDKPQTYRRFIKSCSVEQNFEMRVGCTRDVIVISGLPANTSTERLDILDDERRVTGFSIIGGEHRLTNYKSVTTVHRFEKENRIWTVVLESYVVDMPEGDSEDDTRMFADTVVKLNLQKLATVAEAMARNSGDGSGSQVT

SEQ ID NO: 134 - protein, 191 aa, Artificial Sequence
  synthetic bromoxynil-responsive PYR/PYL receptor mutant clone #74A-4
MPSELTPEERSELKNSIAEFHTYQLDPGSCSSLHAQRIHAPPELVWSIVRRFDKPQTYRRFIKSCSVEQNFEMRVGCTRDVIVISGLPANTSTERLDILDDGRRVTGFSIIGGEHRLTNYKSVTAVHRFEKENRIWTVVLDSYVVDMPEGNSEDDTRMFADTVVKLNLQKLATVAEAMARNSGDGSGSQVT

SEQ ID NO: 135 - protein, 191 aa, Artificial Sequence
  synthetic bromoxynil-responsive PYR/PYL receptor mutant clone #74A-13
MPSELTPEERSELKNSIAEFHTYQLDPGSCSSLHAQRIHAPPELVWSIVRRFDKPQTYRHFIKSCSVEQNFEMRVGCTRDVNVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 136 - protein, 191 aa, Artificial Sequence
  synthetic bromoxynil-responsive PYR/PYL receptor mutant clone #74A-2
MPSELTPEERSELKNSIAEFHTYQLDPGSCSSLHAQRIHAPPELVWSIVRRFDKPQTYRHFIKSCSVEQNFEMRVGCTRDVIVISGLPANTTTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 137 - protein, 191 aa, Artificial Sequence; identical to SEQ ID NO: 136
  synthetic bromoxynil-responsive PYR/PYL receptor mutant clone #74A-15

SEQ ID NO: 138 - protein, 191 aa, Artificial Sequence
  synthetic bromoxynil-responsive PYR/PYL receptor mutant clone #74B-1
MPSELTPEERSELKNSIAEFYTYQLDPGSCSSLHAQRIHAPPELVWSIVRRFDKPQTYRRFIKSCSVEQNFEMRVGCTRDVIVISGLPANTTTERLDILDDERRVTGFSIIGGEHKLTNYKSVTTVHRFEKENRIWTVVLESYVVDMPEGNSEDDTRMFADTVVKLNLQKLATVAEAMARNSGDGSGSQVT

SEQ ID NO: 139 - protein, 191 aa, Artificial Sequence
  synthetic bromoxynil-responsive PYR/PYL receptor mutant clone #74B-7
MPSELTPEERSELKNSIAEFHTYQLDPGSCSSLHAQRIHALPELVWSIVRRFDKPQTYRRFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 140 - protein, 191 aa, Artificial Sequence
  synthetic dichlobenil-responsive PYR/PYL receptor mutant clone #68-1
MPSELTPEERSELKNSIAEFHTYQLDPGSCSSLHAQRIHAPPELVWSIVRRFDKPQTYKHFIKSCSVEQNFEMRVGCTRDVIVISGLPANTSTDRLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 141 - protein, 191 aa, Artificial Sequence
  synthetic dichlobenil-responsive PYR/PYL receptor mutant clone #68-2
MPSELTPEERSELKNSIAEFHTYQLDPGSCSSLHAQQIHAPPELVWSIVRRFDKPQTYKHFIKSCSVEQNSEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 142 - protein, 191 aa, Artificial Sequence
  synthetic dichlobenil-responsive PYR/PYL receptor mutant clone #68-8
MPSELTPEERSELKNSIAEFHTYQLGPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 143 - protein, 191 aa, Artificial Sequence
  synthetic dichlobenil-responsive PYR/PYL receptor mutant clone #68-3
MPSELTPEERSELKNSIAEFHTYQLDLGSCSSLHAQRIHAPPELVWSIVRRFDKPQTYRHFINSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 144 - protein, 191 aa, Artificial Sequence
  synthetic dichlobenil-responsive PYR/PYL receptor mutant clone #68-17
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 145 - protein, 191 aa, Artificial Sequence
  synthetic benoxacor-responsive PYR/PYL receptor mutant clone #129-2
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTYYKSVTTVHRFEKENRIWTVVLESYVVDMPEGNSEDDTRMFADTVVKLNLQKLATVAEAMARNSGDGSGSQVT

SEQ ID NO: 146 - protein, 191 aa, Artificial Sequence
  synthetic benoxacor-responsive PYR/PYL receptor mutant clone #127-1
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSTIGGDHRLTNYKSVTTVHRFEKENRIWTMVLESYVVDMPEGNSEDDTRMFADTVVKLNLQKLATVAEAMARNSGDGSGSQVT

SEQ ID NO: 147 - protein, 191 aa, Artificial Sequence
  synthetic acibenzolar-S-methyl (BTH)-responsive PYR/PYL receptor mutant clone #BTH-1
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEYRLTNYKSVTTVHRFEKENRIWTVVLESYVVDMPEGNSEDDTRMSADTVVKLNLQKLATVAEAMARNSGDGSGSQVT

SEQ ID NO: 148 - protein, 191 aa, Artificial Sequence
  synthetic acibenzolar-S-methyl (BTH)-responsive PYR/PYL receptor mutant clone #BTH-9
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMLADTVVKLNLQKLATVAEAMARNSGDGSGSQVT

SEQ ID NO: 149 - protein, 36 aa, Artificial Sequence; identical to SEQ ID NO: 120
  synthetic modified PYR/PYL receptor consensus sequence with K59 mutation

SEQ ID NO: 150 - protein, 11 aa, Artificial Sequence
  synthetic modified PYR/PYL receptor consensus sequence with Y120 mutation
GGXHRLXNXXS

SEQ ID NO: 151 - protein, 13 aa, Artificial Sequence
  synthetic modified PYR/PYL receptor consensus sequence with I110 mutation
XXGGXHRLXNYXS

SEQ ID NO: 152 - protein, 36 aa, Artificial Sequence; identical to SEQ ID NO: 120
  synthetic modified PYR/PYL receptor consensus sequence with P42 mutation

SEQ ID NO: 153 - protein, 36 aa, Artificial Sequence; identical to SEQ ID NO: 120
  synthetic modified PYR/PYL receptor consensus sequence with S47 mutation

SEQ ID NO: 154 - protein, 36 aa, Artificial Sequence; identical to SEQ ID NO: 120
  synthetic modified PYR/PYL receptor consensus sequence with K59 and I110 mutations

SEQ ID NO: 155 - protein, 36 aa, Artificial Sequence; identical to SEQ ID NO: 120
  synthetic modified PYR/PYL receptor consensus sequence with K59 and S47 mutations

SEQ ID NO: 156 - protein, 36 aa, Artificial Sequence
  synthetic modified PYR/PYL receptor consensus sequence with H60 mutation
CXSXXXXXXXAPXXXXWXXXXXFXXPXXXXRFXXXC

SEQ ID NO: 157 - protein, 25 aa, Artificial Sequence
  synthetic modified PYR/PYL receptor consensus sequence with S92 mutation
GXXRXVXXXSXXPAXXTXEXLXXXD

SEQ ID NO: 158 - protein, 31 aa, Artificial Sequence
  synthetic modified PYR/PYL receptor consensus sequence with E140 mutation
XSXXVDXPXGNXXXXTXXFXXXXXXXNLXXL

SEQ ID NO: 159 - protein, 36 aa, Artificial Sequence; identical to SEQ ID NO: 156
  synthetic modified PYR/PYL receptor consensus sequence with K59 and H60 mutations

SEQ ID NO: 160 - protein, 25 aa, Artificial Sequence
  synthetic modified PYR/PYL receptor consensus sequence with E94 mutation
GXXRXVXXXSXXPAXXSXDXLXXXD

SEQ ID NO: 161 - protein, 11 aa, Artificial Sequence
  synthetic modified PYR/PYL receptor consensus sequence with K59 and N119 mutations
GGXHRLXYYXS

SEQ ID NO: 162 - protein, 11 aa, Artificial Sequence
  synthetic modified PYR/PYL receptor consensus sequence with H115 mutation
GGXYRLXNYXS

SEQ ID NO: 163 - protein, 31 aa, Artificial Sequence
  synthetic modified PYR/PYL receptor consensus sequence with F159 mutation
ESXXVDXPXGNXXXXTXXXXXXXXXXNLXXL

SEQ ID NO: 164 - protein, 191 aa, Artificial Sequence
  synthetic acibenzolar-S-methyl (BTH)-responsive PYR/PYL receptor mutant clone #BTH-An7
MPSELTPEERSELKNSIAEFHTYRLDPGSCSSLHAQRIHAPPELVWSIVRRFDKPQTYRHFIKSCSVEQNFEMRVGCTRDVTVISGLPANTSTERLDILDDERRVTGFSIIGGEHRLTNYKSVTTVHRFEKENRIWTVVLESYVVDMPEGNSEDDTRMLAGTVVKLNLQKLATVAEAMARNSGDGSGSQVT

SEQ ID NO: 165 - protein, 191 aa, Artificial Sequence
  synthetic fenhexamid-responsive PYR/PYL receptor mutant clones #27B-1 and #27B-8
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLNILDDERRVTGFSIIGGEHRLTNHKSVTTVHRFEKENRIWTVVLESYVVDMPEGNSEDDTRMFADTIVKLNLQKLTTVAEAMARNSGDGSGSQVT

SEQ ID NO: 166 - protein, 191 aa, Artificial Sequence
  synthetic fenhexamid-responsive PYR/PYL receptor mutant clone #27B-2
MPSELTPEERSELKNSIAEFHTYQLDPGSCSSLHAQRIHAPSEFVWSIVRRFDKPQTYRHFIKSCSVEQNFEMRVGCTRDVIVISGLPANTSTERLDILDDERRVTGFSIIGGEHRLTNHKSVTTVHRFEKENRIWTMVLESYVVDMPEGNSEDDTRIFADTVVKLNLQKLATVAEAMARNSGDGSGSQVT

SEQ ID NO: 167 - protein, 191 aa, Artificial Sequence
  synthetic fenhexamid-responsive PYR/PYL receptor mutant clone #27B-3
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLDILDDERRVTGFSIIGGEHRLTNHKSITTVHRFEKENRIWTVILESYVVDMPEGNSEDDTRIFADTVVKLNLQKLATVAEAMARNSGDGSGSQVT

SEQ ID NO: 168 - protein, 191 aa, Artificial Sequence
  synthetic fenhexamid-responsive PYR/PYL receptor mutant clone #27B-4
MPSELTPEERSELKNSIAEFHTYQLDPGSCSSLHAQRIHAPPELVWPIIRRFDKPQTYRHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRIFADTVVKLNLQKLATVAETMARNSGDGSGSQVT

SEQ ID NO: 169 - protein, 191 aa, Artificial Sequence
  synthetic fenhexamid-responsive PYR/PYL receptor mutant clone #27B-7
MPSELTPEERSELKNSIAEFHTYQLDPGSCSSLHAQRIHAPPELVWSIVRRFDKPQTYRHFIKSCSVEQNFEMRVGCTRDMIVISGLPANTSTERLDILDDERRVTGFSIIGGEHRLTNCKSVTTVHRFEKENRIWTVVLESYVVDMPEGNSEDDTRIFADTIVKLNLQKLATVAEAMARNSGDGSGSQVT

SEQ ID NO: 170 - protein, 191 aa, Artificial Sequence
  synthetic fenhexamid-responsive PYR/PYL receptor mutant clone #27C-1
MPSELTPEERSELKNSIAEFHTYQLDLGSCSSLHAQRIHAPSELVWSIVRRFDKPQTYRHFIKSCSVEQNFEMRVGCTRDVIVISGLPANTSTERLNILDDERRVTGFSIIGGEHRLTNHKSVTTVHRFEKENRIWTVVLESYVVDMPEGNSEDDTRIFADTVVKLNLQKLAAVAEAMARNSGDGSGSQVT

SEQ ID NO: 171 - protein, 191 aa, Artificial Sequence
  synthetic fenhexamid-responsive PYR/PYL receptor mutant clone #27C-2
MPSELTPEERSELKNSIAEFHTYQLDPGSCSSLHAQRIHAPSELVWSIVRRFDKPQTYRHFIKSCSVEQNFEMCVGCTRDVIVISGLPANTSTERLDILDDERRVTGFSIIGGEHRLTNHKSVTTVHRFEKENRIWTVVLESYVVDMPEGNSEDDTRIFADTVVKLNLQKLATVAEAMARNSGDGSGSQVT

SEQ ID NO: 172 - protein, 191 aa, Artificial Sequence
  synthetic fenhexamid-responsive PYR/PYL receptor mutant clone #27C-3
MPSELTPEERSELKNSIAEFHTYQLDPGNCSSLHAQRIHAPPELVWSIVRRFDKPQTYRHFIKSCSVEQNFEMRVGCTRDVIVISGLPANTSTERLNILDDERRVTGFSIIGGEHRLTNHKSVTTVHRFEKENRIWTVVLESYVVDMPEGNSEDDTRMFADTIVKLNLQKLTTVAEAMARNSGDGSGSQVT

SEQ ID NO: 173 - protein, 191 aa, Artificial Sequence
  synthetic fenhexamid-responsive PYR/PYL receptor mutant clone #27C-5
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLDILDDERRVTGFSIIGGEHRLTNHKSITTVHRFEKENRIWTVILESYVVDMPEGNSEDDTRIFADTIVKLNLQKLATVAEAMARNSGDGSGSQVT

SEQ ID NO: 174 - protein, 191 aa, Artificial Sequence
  synthetic fenhexamid-responsive PYR/PYL receptor mutant clone #27C-16
MPSELTPEERSELKNSIAEFHTYQLDPGSCSSLHAQRIHAPPELVWSIVRRFDKPQTYRHFIKSCSVEQNFEMRVGCTRDMIVISGLPANTSTERLDILDDERRVTGFSIIGGEHRLTNHKSVTTVHRFEKENRIWTVVLESYVVDMPEGNSEDDTRIFADTIVKLNLQKLATVAEAMARNSGDGSGSQVT

SEQ ID NO: 175 - protein, 191 aa, Artificial Sequence
  synthetic fenhexamid-responsive PYR/PYL receptor mutant clone #27C-18
MPSELTPEERSKLKNSIAEFHTYQLDPGSCSSLHAQRIHAPPELVWSIVRRFDKPQTYRHFIKSCSVEQNFEMRIGCTRDVIVISGLPANTSTERLNILDDERRVTGFSIIGGEHRLTNHKSVTTVHRFEKENRIWTVVLESYVVDMPEGNSEDDTRMFADTIVKLNLQKLTTVAEAMARNSGDGSGSQVT

SEQ ID NO: 176 - protein, 191 aa, Artificial Sequence
  synthetic fenhexamid-responsive PYR/PYL receptor mutant clone #27C-19
MPSELTPEERSELKNSIAEFHTYQLDPGSCSSFHAQRIHAPSELVWSIVRRFDKPQTYRHFIKSCSVEQNFEMRVGCTRDVIVISGLPANTSTERLDILDDERRVTGFSIIGGEHRLTNHKSITTVHRFEKENRIWTVVLESYVVDMPEGNSEDDTRIFADTVVKLNLQKLATVAEAMARNSGDGSGSQVT

SEQ ID NO: 177 - protein, 191 aa, Artificial Sequence
  synthetic fenhexamid-responsive PYR/PYL receptor mutant clone #27C-20
MPSELTPEERSELKNSIAEFHTYQLDPGSCSSLHAQRIHAPSELVWSIVRRFDKPQTYRHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRIFADTIVKLNLQKLATIAEAMARNSGDGSGSQVT

SEQ ID NO: 178 - protein, 191 aa, Artificial Sequence
  synthetic fenhexamid-responsive PYR/PYL receptor mutant clone #27C-21
MPSELTPEEQSELKNSIAEFHTYQLDPGSCSSLHAQRIHAPSELVWSIVRRFDKPQTYRHFIKSCSVEQNFEMRVGCTRDVIVISGLPANTSTERLNILDDERRVTGFSIIGGEHRLTNHKSVTTVHRFEKENRIWTVVLESYVVDMPEGNSEDDTRMFADTIVKLNLQKLTTVAEAMARNSGDGSGSQVT

SEQ ID NO: 179 - protein, 6 aa, Artificial Sequence
  synthetic N-terminus 6X-histidine tag
HHHHHH

(19) United States
(12) Patent Application Publication
(10) Pub. No.: US 2016/0312240 A1
(43) Pub. Date: Oct. 27, 2016

(54) MODIFIED PYR/PYL RECEPTORS ACTIVATED BY ORTHOGONAL LIGANDS

(71) Applicant: The Regents of the University of California, Oakland, CA (US)

(72) Inventors: Sean R. Cutler, Riverside, CA (US); Sang-Youl Park, Riverside, CA (US)

(21) Appl. No.: 15/145,451

(22) Filed: May 3, 2016

Related U.S. Application Data
(63) Continuation of application No. 13/095,796, filed on Apr. 27, 2011, now abandoned.
(60) Provisional application No. 61/434,407, filed on Jan. 19, 2011.
(60) Provisional application No. 61/328,999, filed on Apr. 28, 2010.

(51) Int. Cl. C12N 15/82; C07K 14/415; A01N 37/24; G01N 33/68

(52) U.S. Cl. CPC C12N 15/8271; G01N 33/6872; C07K 14/415; A01N 37/24; G01N 2333/415

(57) ABSTRACT

The present invention provides polynucleotides encoding mutated PYR/PYL receptor polypeptides that are agonized by chemicals, such as bromoxynil, chloroxynil, ioxynil, coumatetralyl, dichlobenil, fenhexamid, benoxacor, and BTH, that do not agonize wild-type PYR/PYL receptor polypeptides, and expression cassettes and plants comprising the polynucleotides. Particular embodiments of the invention provide polynucleotides encoding mutated PYR/PYL receptor polypeptides having a mutation in the ligand-binding pocket of the PYR/PYL receptor polypeptide.

### CROSS-REFERENCE TO RELATED APPLICATIONS

The present patent application claims priority to U.S. patent application Ser. No. 13/095,796, filed Apr. 27, 2011, which claims the benefit of U.S. Provisional Patent Application No. 61/328,999, filed Apr. 28, 2010, and of U.S. Provisional Patent Application No. 61/434,407, filed Jan. 19, 2011, the contents of each of which is incorporated by reference herein for all purposes.

### STATEMENT AS TO RIGHTS TO INVENTIONS MADE UNDER FEDERALLY SPONSORED RESEARCH AND DEVELOPMENT

This invention was made with Government support under Grant No. 10S0820508, awarded by the National Science Foundation. The Government has certain rights in this invention.

### REFERENCE TO SEQUENCE LISTING

This application includes a Sequence Listing as a text file named “081906-1009646-205420US-SEQLIST.txt” created Mar. 3, 2016, and containing 306,220 bytes. The material contained in this text file is incorporated by reference in its entirety for all purposes.

### BACKGROUND OF THE INVENTION

Rising temperatures and lessening fresh water supplies are two forms of environmental stress, also called abiotic stress, that lower the amount of food produced by agriculture. A key regulator of abiotic stress tolerance is the plant hormone abscisic acid (ABA), which is synthesized by plants in response to various abiotic stresses and orchestrates adaptive responses that enhance plant survival (Cutler, S. et al., Annual Review of Plant Biology (2009); Nambara, E. et al., Annual Review of Plant Biology 56:165-185 (2005)). Crop plants engineered to have increased ABA sensitivity show improved yield under conditions of drought (Wang, Y. et al., Plant J 43:413-424 (2005)). Moreover, the direct application of ABA or ABA analogs to plants in the field has been shown to improve water use efficiency (Hawkins, A. F. et al., Plant Growth Regulators for Agricultural and Amenity Use (British Crop Protection Council) (1987); Kreeb, K. H. et al., Structural and Functional Responses to Environmental Stresses (Balogh Scientific Books) (1989)); however, ABA has not been successfully commercialized for this use given its complicated production routes and high cost.

Interestingly, numerous fungicides and insecticides have shown stress-tolerance “side-effects” of unknown mechanism and have been commercialized for stress-tolerance uses, which demonstrates the strong interest in, and recognized need for chemical methods to control stress tolerance (Asrar, J. et al., In US 2009/0270254 Al (U.S.A., Monsanto Technology) (2003); Beckers, G. J. M. et al., Current Opinion in Plant Biology 10:425-431 (2007); Schulz, A. et al., In US 2007/0124839 Al (U.S.A., Bayer Crop Sciences) (2006)). An important driver of this interest has been the realization that the dramatic increases in corn yield achieved over last 100 years can be attributed largely to improvements in abiotic stress tolerance of new high-yielding corn varieties (Duvick, D. N. et al., Crop Science 39:1622-1630 (1999); Tollenaar, M. et al., Field Crops Research 75:161-169 (2002); Tollenaar, M. et al., Crop Sci 39:1597-1604 (1999)). Because ABA is recognized as the critical hormonal regulator of plant stress physiology, there is intense interest in modulating the ABA pathway in crops. One possible point at which to control the ABA signaling pathway is receptor proteins, which in principle would allow both chemical and genetic modulation of ABA signaling and stress tolerance.

Recently a new family of ABA receptors, the Pyrabactin resistance/PYR-like (“PYR/PYL”) family, was identified as a modulator of ABA signaling (Park, S. Y. et al., Science 324:1068-1071 (2009)). The over-expression of the ABA receptor PYL5 confers drought tolerance on Arabidopsis plants (Santiago, J. et al., The Plant Journal 9999 (2009)), validating this new receptor family as a key target for control of plant stress tolerance. However, gene over-expression can have adverse yield consequences, which are referred to as “yield drag”. Yield drag is thought to occur because the unregulated activation of stress tolerance pathways, which is associated with slowed growth, occurs under normal conditions (i.e. in the absence of drought or other stressors). One way to gain regulated control of ABA signaling is to develop chemical agents that activate ABA receptors (i.e. agonists). These can be applied to plants once drought or other stress conditions have ensued, which allows for selective protection in adverse conditions. This allows the benefits of stress tolerance to be realized without lowering yield under ideal growth conditions.

In principle, ABA could be used as an agonist to realize these advantages. However, it is a natural product that is costly to make and rapidly degraded by both UV photo-isomerization and metabolic inactivation. It also has physiological effects in mammals that could conceivably affect its suitability for use as an agrochemical (Guri, A. J. et al., Clin Nutr. (2010)). Therefore, a cheap, environmentally stable and non-toxic molecule would be an ideal reagent with which to control ABA signaling.

### BRIEF SUMMARY OF THE INVENTION

The present invention provides for plants (or a plant cell, seed, flower, leaf, fruit, or other plant part from such plants or processed food or food ingredient from such plants) comprising a heterologous expression cassette, the expression cassette comprising a promoter operably linked to a polynucleotide encoding a mutated PYR/PYL receptor polypeptide, wherein the mutated PYR/PYL receptor polypeptide is agonized by a chemical when the chemical is contacted to the mutated PYR/PYL receptor polypeptide and wherein the chemical does not significantly agonize a wild-type PYR/PYL receptor polypeptide when the chemical is contacted to the wild-type PYR/PYL receptor polypeptide.

In some embodiments, the chemical comprises a fungicide, an herbicide, a pesticide, a nematicide, a plant activator, a synergist, an herbicide safener, a plant growth regulator, an insect repellant, or a fertilizer.

In some embodiments, the chemical is selected from the group consisting of bromoxynil, chloroxynil, ioxynil, coumatetralyl, dichlobenil, fenhexamid, benoxacor, and BTH (acibenzolar-s-methyl).

In some embodiments, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan.

In some embodiments, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the chemical is bromoxynil, chloroxynil, ioxynil, dichlobenil, benoxacor, or fenhexamid.

In some embodiments, wherein the mutated PYR/PYL receptor polypeptide is agonized by bromoxynil, chloroxynil, or ioxynil when the bromoxynil, chloroxynil, or ioxynil is contacted to the mutated PYR/PYL receptor polypeptide, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one additional mutation at an amino acid corresponding to positions 21, 41, 50, 57, 60, 82, 92, 102, 116, 125, 141, and/or 151 in PYR1 (SEQ ID NO:1) wherein the mutation is selected from H21Y, P41L, R50G, T57A, H60R, I82N, S92T, E102G, R116K, T125A, E141Q, E141D, N151D, or combinations thereof.

In some embodiments, wherein the mutated PYR/PYL receptor polypeptide is agonized by fenhexamid when the fenhexamid is contacted to the mutated PYR/PYL receptor polypeptide, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one additional mutation at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in PYR1 (SEQ ID NO:1) wherein the mutation is selected from R10Q, E12G, E12K, L25R, P27L, S29N, L33F, P42S, E43G, L44F, S47P, V49I, R74C, V75I, V81M, D97N, I110S, Y120C, Y120H, V123I, T124M, N133D, V138M, V139I, V144A, E154G, M158I, V163I, A172T, T173A, V174I, A177T or combinations thereof.

In some embodiments, wherein the mutated PYR/PYL receptor polypeptide is agonized by fenhexamid when the fenhexamid is contacted to the mutated PYR/PYL receptor polypeptide, the mutated PYR/PYL receptor polypeptide comprises mutations at amino acids corresponding to positions 59, 120, and 158 in PYR1 (SEQ ID NO:1) wherein the mutations are K59R, Y120H, and M158I. In some embodiments, the mutated PYR/PYL receptor polypeptide further comprises isoleucine residues at the amino acid positions corresponding to positions 62 and 110 in PYR1 (SEQ ID NO:1).

In some embodiments, wherein the mutated PYR/PYL receptor polypeptide is agonized by dichlobenil when the dichlobenil is contacted to the mutated PYR/PYL receptor polypeptide, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one additional mutation at an amino acid corresponding to positions 27 and/or 63 in PYR1 (SEQ ID NO:1) wherein the mutation is selected from P27L, K63N, or combinations thereof.

In some embodiments, wherein the mutated PYR/PYL receptor polypeptide is agonized by dichlobenil when the dichlobenil is contacted to the mutated PYR/PYL receptor polypeptide, the mutated PYR/PYL receptor polypeptide comprises at least one mutation at an amino acid corresponding to positions 26, 37, 71, and/or 94 in PYR1 (SEQ ID NO:1) wherein the mutation is selected from D26G, R37Q, F71S, E94D, or combinations thereof.

In some embodiments, wherein the mutated PYR/PYL receptor polypeptide is agonized by benoxacor when the benoxacor is contacted to the mutated PYR/PYL receptor polypeptide, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises a mutation at an amino acid corresponding to position 119 in PYR1 (SEQ ID NO:1) wherein the mutation is N119Y.

In some embodiments, wherein the mutated PYR/PYL receptor polypeptide is agonized by benoxacor when the benoxacor is contacted to the mutated PYR/PYL receptor polypeptide, the mutated PYR/PYL receptor polypeptide comprises at least one mutation at an amino acid corresponding to positions 110, 114, and/or 138 in PYR1 (SEQ ID NO:1) wherein the mutation is selected from I110T, E114D, V138M, or combinations thereof.

In some embodiments, wherein the mutated PYR/PYL receptor polypeptide is agonized by BTH when the BTH is contacted to the mutated PYR/PYL receptor polypeptide, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one mutation at an amino acid corresponding to positions 24, 82, 159, and/or 161 in PYR1 (SEQ ID NO:1) wherein the mutation is selected from Q24R, I82T, F159L, D161G, or combinations thereof.

In some embodiments, wherein the mutated PYR/PYL receptor polypeptide is agonized by BTH when the BTH is contacted to the mutated PYR/PYL receptor polypeptide, the mutated PYR/PYL receptor polypeptide comprises at least one mutation at an amino acid corresponding to positions 115 and/or 159 in PYR1 (SEQ ID NO:1) wherein the mutation is selected from H115Y, F159S, F159L, or combinations thereof.

In some embodiments, the mutated PYR/PYL receptor polypeptide is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, or 119 and comprises one or more mutations as described herein.

In some embodiments, the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:131-139 (i.e., any of SEQ ID NO:131, 132, 133, 134, 135, 136, 137, 138, or 139) and the mutated PYR/PYL receptor polypeptide is agonized by bromoxynil, chloroxynil, or ioxynil when the bromoxynil, chloroxynil, or ioxynil is contacted to the mutated PYR/PYL receptor polypeptide.

In some embodiments, the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:124-130 or 165-178 (i.e., any of SEQ ID NO:124, 125, 126, 127, 128, 129, 130, 165, 166, 167, 168, 169, 170, 171, 172, 173, 174, 175, 176, 177, or 178) and the mutated PYR/PYL receptor polypeptide is agonized by fenhexamid when the fenhexamid is contacted to the mutated PYR/PYL receptor polypeptide.

In some embodiments, the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:140-144 (i.e., any of SEQ ID NO:140, 141, 142, 143, or 144) and the mutated PYR/PYL receptor polypeptide is agonized by dichlobenil when the dichlobenil is contacted to the mutated PYR/PYL receptor polypeptide.

In some embodiments, the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:145 or 146 and the mutated PYR/PYL receptor polypeptide is agonized by benoxacor when the benoxacor is contacted to the mutated PYR/PYL receptor polypeptide.

In some embodiments, the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:147, 148, or 164, and the mutated PYR/PYL receptor polypeptide is agonized by BTH (acibenzolar-s-methyl) when the BTH is contacted to the mutated PYR/PYL receptor polypeptide.

In some embodiments, the mutated PYR/PYL receptor polypeptide comprises at least one mutation at an amino acid residue comprising the ligand-binding pocket of the PYR/PYL receptor polypeptide.

In some embodiments, the plant has improved abiotic stress tolerance when contacted with the chemical as compared to a plant lacking the expression cassette.

The present invention also provides for methods of improving abiotic stress tolerance in such plants as described above by contacting the plant with a chemical selected from the group consisting of bromoxynil, chloroxynil, ioxynil, coumatetralyl, dichlobenil, fenhexamid, benoxacor, and BTH (acibenzolar-s-methyl).

The present invention also provides for polypeptides comprising the mutated PYR/PYL receptor polypeptides of the invention (e.g., as described herein). In some embodiments, a polypeptide comprises a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:124-148 or 164-178 (i.e., any of SEQ ID NO:124, 125, 126, 127, 128, 129, 130, 131, 132, 133, 134, 135, 136, 137, 138, 139, 140, 141, 142, 143, 144, 145, 146, 147, 148, 164, 165, 166, 167, 168, 169, 170, 171, 172, 173, 174, 175, 176, 177, or 178).

The present invention further provides for polynucleotides encoding one or more of the mutated PYR/PYL receptor polypeptides of the invention (e.g., as described herein). In some embodiments, a polynucleotide encodes one or more mutated PYR/PYL receptor polypeptides that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:124-148 or 164-178 (i.e., any of SEQ ID NO:124, 125, 126, 127, 128, 129, 130, 131, 132, 133, 134, 135, 136, 137, 138, 139, 140, 141, 142, 143, 144, 145, 146, 147, 148, 164, 165, 166, 167, 168, 169, 170, 171, 172, 173, 174, 175, 176, 177, or 178). The present invention further provides for isolated nucleic acids comprising a polynucleotide sequence encoding one or more of the mutated PYR/PYL receptor polypeptides of the invention (e.g., as described herein).

The present invention also provides for methods of making a mutated PYR/PYL receptor polypeptide that is agonized by a chemical when the chemical is contacted to the mutated PYR/PYL receptor polypeptide, wherein the chemical does not significantly agonize a wild-type PYR/PYL receptor polypeptide when the chemical is contacted to the wild-type PYR/PYL receptor polypeptide, the method comprising
(a) mutagenizing the wild-type PYR/PYL receptor polypeptide;
(b) contacting one or more mutated PYR/PYL receptor polypeptides with the chemical; and
(c) determining whether the chemical activates the one or more mutated PYR/PYL receptor polypeptides, wherein activation identifies the one or more mutated PYR/PYL receptor polypeptides as being agonized by the chemical.

In some embodiments, the method further comprises, prior to step (b), screening the chemical to determine whether the chemical binds to the wild-type PYR/PYL receptor polypeptide prior to contacting the one or more mutated PYR/PYL receptor polypeptides with the chemical.

In some embodiments, determining step (c) of the method comprises contacting the chemical to a cell comprising a two-hybrid system, wherein the two-hybrid system detects interaction of the mutated PYR/PYL receptor polypeptide with a type 2 protein phosphatase (PP2C), and wherein chemical-specific interaction of the mutated PYR/PYL receptor polypeptide with the PP2C identifies the mutated PYR/PYL receptor polypeptide as being agonized by the chemical.

In some embodiments, the chemical of the method comprises a fungicide, an herbicide, a pesticide, a nematicide, a plant activator, a synergist, an herbicide safener, a plant growth regulator, an insect repellant, or a fertilizer.

In some embodiments, the chemical of the method is selected from the group consisting of bromoxynil, chloroxynil, ioxynil, coumatetralyl, dichlobenil, fenhexamid, benoxacor, and BTH (acibenzolar-s-methyl).

The present invention also provides for expression cassettes comprising a promoter operably linked to a polynucleotide encoding a mutated PYR/PYL receptor polypeptide, wherein the mutated PYR/PYL receptor polypeptide is agonized by a chemical when the chemical is contacted to the mutated PYR/PYL receptor polypeptide and wherein the chemical does not significantly agonize a wild-type PYR/PYL receptor polypeptide when the chemical is contacted to the wild-type PYR/PYL receptor polypeptide.

In some embodiments, the expression cassette comprises a promoter operably linked to a polynucleotide encoding a mutated PYR/PYL receptor polypeptide, wherein the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan. In some embodiments, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the chemical is bromoxynil, chloroxynil, ioxynil, dichlobenil, benoxacor, or fenhexamid.

In some embodiments, the expression cassette comprises a promoter operably linked to a polynucleotide encoding a mutated PYR/PYL receptor polypeptide, wherein the mutated PYR/PYL receptor polypeptide is agonized by bromoxynil, chloroxynil, or ioxynil when the bromoxynil, chloroxynil, or ioxynil is contacted to the mutated PYR/PYL receptor polypeptide, and the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one additional mutation at an amino acid corresponding to positions 21, 41, 50, 57, 60, 82, 92, 102, 116, 125, 141, and/or 151 in PYR1 (SEQ ID NO:1) wherein the mutation is selected from H21Y, P41L, R50G, T57A, H60R, 182N, S92T, E102G, R116K, T125A, E141Q, E141D, N151D, or combinations thereof.

In some embodiments, the expression cassette comprises a promoter operably linked to a polynucleotide encoding a mutated PYR/PYL receptor polypeptide, wherein the mutated PYR/PYL receptor polypeptide is agonized by fenhexamid when the fenhexamid is contacted to the mutated PYR/PYL receptor polypeptide, and the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one additional mutation at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in PYR1 (SEQ ID NO:1) wherein the mutation is selected from R10Q, E12G, E12K, L25R, P27L, S29N, L33F, P42S, E43G, L44F, S47P, V491, R74C, V751, V81M, D97N, I110S, Y120C, Y120H, V1231, T124M, N133D, V138M, V139I, V144A, E154G, M158I, V163I, A172T, T173A, V174I, A177T or combinations thereof.

In some embodiments, the expression cassette comprises a promoter operably linked to a polynucleotide encoding a mutated PYR/PYL receptor polypeptide, wherein the mutated PYR/PYL receptor polypeptide is agonized by fenhexamid when the fenhexamid is contacted to the mutated PYR/PYL receptor polypeptide, and the mutated PYR/PYL receptor polypeptide comprises mutations at amino acids corresponding to positions 59, 120, and 158 in PYR1 (SEQ ID NO:1) wherein the mutations are K59R, Y120H, and M158I. In some embodiments, the mutated PYR/PYL receptor polypeptide further comprises isoleucine residues at the amino acid positions corresponding to positions 62 and 110 in PYR1 (SEQ ID NO:1).

In some embodiments, the expression cassette comprises a promoter operably linked to a polynucleotide encoding a mutated PYR/PYL receptor polypeptide, wherein the mutated PYR/PYL receptor polypeptide is agonized by dichlobenil when the dichlobenil is contacted to the mutated PYR/PYL receptor polypeptide, and the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one additional mutation at an amino acid corresponding to positions 27 and/or 63 in PYR1 (SEQ ID NO:1) wherein the mutation is selected from P27L, K63N, or combinations thereof.

In some embodiments, the expression cassette comprises a promoter operably linked to a polynucleotide encoding a mutated PYR/PYL receptor polypeptide, wherein the mutated PYR/PYL receptor polypeptide is agonized by dichlobenil when the dichlobenil is contacted to the mutated PYR/PYL receptor polypeptide, and the mutated PYR/PYL receptor polypeptide comprises at least one mutation at an amino acid corresponding to positions 26, 37, 71, and/or 94 in PYR1 (SEQ ID NO:1) wherein the mutation is selected from D26G, R37Q, F71S, E94D, or combinations thereof.

In some embodiments, the expression cassette comprises a promoter operably linked to a polynucleotide encoding a mutated PYR/PYL receptor polypeptide, wherein the mutated PYR/PYL receptor polypeptide is agonized by benoxacor when the benoxacor is contacted to the mutated PYR/PYL receptor polypeptide, and the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises a mutation at an amino acid corresponding to position 119 in PYR1 (SEQ ID NO:1) wherein the mutation is N119Y.

In some embodiments, the expression cassette comprises a promoter operably linked to a polynucleotide encoding a mutated PYR/PYL receptor polypeptide, wherein the mutated PYR/PYL receptor polypeptide is agonized by benoxacor when the benoxacor is contacted to the mutated PYR/PYL receptor polypeptide, and the mutated PYR/PYL receptor polypeptide comprises at least one mutation at an amino acid corresponding to positions 110, 114, and/or 138 in PYR1 (SEQ ID NO:1) wherein the mutation is selected from I110T, E114D, V138M, or combinations thereof.

In some embodiments, the expression cassette comprises a promoter operably linked to a polynucleotide encoding a mutated PYR/PYL receptor polypeptide, wherein the mutated PYR/PYL receptor polypeptide is agonized by BTH when the BTH is contacted to the mutated PYR/PYL receptor polypeptide, and the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan, and the mutated PYR/PYL receptor polypeptide further comprises at least one mutation at an amino acid corresponding to positions 24, 82, 159, and/or 161 in PYR1 (SEQ ID NO:1) wherein the mutation is selected from Q24R, I82T, F159L, D161G, or combinations thereof.

In some embodiments, the expression cassette comprises a promoter operably linked to a polynucleotide encoding a mutated PYR/PYL receptor polypeptide, wherein the mutated PYR/PYL receptor polypeptide is agonized by BTH when the BTH is contacted to the mutated PYR/PYL receptor polypeptide, and the mutated PYR/PYL receptor polypeptide comprises at least one mutation at an amino acid corresponding to positions 115 and/or 159 in PYR1 (SEQ ID NO:1) wherein the mutation is selected from H115Y, F159S, F159L, or combinations thereof.

In some embodiments, the expression cassette comprises a promoter operably linked to a polynucleotide encoding a mutated PYR/PYL receptor polypeptide, wherein the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:131-139 (i.e., any of SEQ ID NO:131, 132, 133, 134, 135, 136, 137, 138, or 139) and the mutated PYR/PYL receptor polypeptide is agonized by bromoxynil, chloroxynil, or ioxynil when the bromoxynil, chloroxynil, or ioxynil is contacted to the mutated PYR/PYL receptor polypeptide.

In some embodiments, the expression cassette comprises a promoter operably linked to a polynucleotide encoding a mutated PYR/PYL receptor polypeptide, wherein the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:124-130 or 165-178 (i.e., any of SEQ ID NO:124, 125, 126, 127, 128, 129, 130, 165, 166, 167, 168, 169, 170, 171, 172, 173, 174, 175, 176, 177, or 178) and the mutated PYR/PYL receptor polypeptide is agonized by fenhexamid when the fenhexamid is contacted to the mutated PYR/PYL receptor polypeptide.

In some embodiments, the expression cassette comprises a promoter operably linked to a polynucleotide encoding a mutated PYR/PYL receptor polypeptide, wherein the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:140-144 (i.e., any of SEQ ID NO:140, 141, 142, 143, or 144) and the mutated PYR/PYL receptor polypeptide is agonized by dichlobenil when the dichlobenil is contacted to the mutated PYR/PYL receptor polypeptide.

In some embodiments, the expression cassette comprises a promoter operably linked to a polynucleotide encoding a mutated PYR/PYL receptor polypeptide, wherein the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:145 or 146 and the mutated PYR/PYL receptor polypeptide is agonized by benoxacor when the benoxacor is contacted to the mutated PYR/PYL receptor polypeptide.

In some embodiments, the expression cassette comprises a promoter operably linked to a polynucleotide encoding a mutated PYR/PYL receptor polypeptide, wherein the mutated PYR/PYL receptor polypeptide has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:147, 148, or 164, and the mutated PYR/PYL receptor polypeptide is agonized by BTH (acibenzolar-s-methyl) when the BTH is contacted to the mutated PYR/PYL receptor polypeptide.

In some embodiments, the expression cassette comprises a promoter operably linked to a polynucleotide encoding a mutated PYR/PYL receptor polypeptide, wherein the mutated PYR/PYL receptor polypeptide comprises at least one mutation at an amino acid residue comprising the ligand-binding pocket of the PYR/PYL receptor polypeptide.

The present invention also provides for expression vectors comprising an expression cassette of the invention (e.g., as described herein).

The present invention further provides for polynucleotide sequences comprising an expression cassette of the invention (e.g., as described herein).

The present invention further provides for methods of producing plants having increased stress tolerance. In some embodiments, the method comprises growing a transgenic plant comprising at least one polynucleotide sequence encoding a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:124-148 or 164-178 (i.e., any of SEQ ID NO:124, 125, 126, 127, 128, 129, 130, 131, 132, 133, 134, 135, 136, 137, 138, 139, 140, 141, 142, 143, 144, 145, 146, 147, 148, 164, 165, 166, 167, 168, 169, 170, 171, 172, 173, 174, 175, 176, 177, or 178), whereby the transgenic plant expresses the mutated PYR/PYL receptor polypeptide.

In some embodiments, the method comprises growing a transgenic plant comprising at least one polynucleotide sequence encoding a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:131-139, whereby the transgenic plant expresses the mutated PYR/PYL receptor polypeptide and the mutated PYR/PYL receptor polypeptide is agonized by bromoxynil, chloroxynil, or ioxynil when the bromoxynil, chloroxynil, or ioxynil is contacted to the mutated PYR/PYL receptor polypeptide.

In some embodiments, the method comprises growing a transgenic plant comprising at least one polynucleotide sequence encoding a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:124-130 or 165-178, whereby the transgenic plant expresses the mutated PYR/PYL receptor polypeptide and the mutated receptor polypeptide is agonized by fenhexamid when the fenhexamid is contacted to the mutated PYR/PYL receptor polypeptide.

In some embodiments, the method comprises growing a transgenic plant comprising at least one polynucleotide sequence encoding a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:140-144, whereby the transgenic plant expresses the mutated PYR/PYL receptor polypeptide and the mutated PYR/PYL receptor polypeptide is agonized by dichlobenil when the dichlobenil is contacted to the mutated PYR/PYL receptor polypeptide.

In some embodiments, the method comprises growing a transgenic plant comprising at least one polynucleotide sequence encoding a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:145-146, whereby the transgenic plant expresses the mutated PYR/PYL receptor polypeptide and the mutated PYR/PYL receptor polypeptide is agonized by benoxacor when the benoxacor is contacted to the mutated PYR/PYL receptor polypeptide.

In some embodiments, the method comprises growing a transgenic plant comprising at least one polynucleotide sequence encoding a mutated PYR/PYL receptor polypeptide that has the sequence of, or is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:147-148 or 164, whereby the transgenic plant expresses the mutated PYR/PYL receptor polypeptide and the mutated PYR/PYL receptor polypeptide is agonized by BTH (acibenzolar-s-methyl) when the BTH is contacted to the mutated PYR/PYL receptor polypeptide.

In some embodiments, the method comprises growing a transgenic plant comprising at least one polynucleotide sequence encoding a mutated PYR/PYL receptor polypeptide that is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:1-119 (i.e., any of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, or 119) and comprises an amino acid X at the position corresponding to position K59 of SEQ ID NO:1, wherein X is alanine, cysteine, aspartic acid, glutamic acid, phenylalanine, glycine, histidine, leucine, methionine, glutamine, arginine, serine, threonine, valie, tyrosine, asparagine, or tryptophan; whereby the transgenic plant expresses the mutated PYR/PYL receptor polypeptide. In some embodiments, the mutated PYR/PYL receptor polypeptide further comprises at least one additional mutation at an amino acid corresponding to positions 21, 41, 50, 57, 60, 82, 92, 102, 116, 125, 141, and/or 151 in SEQ ID NO:1 wherein the mutation is selected from H21Y, P41L, R50G, T57A, H60R, I82N, S92T, E102G, R116K, T125A, E141Q, E141D, N151D, or combinations thereof. In some embodiments, the mutated PYR/PYL receptor polypeptide further comprises at least one additional mutation at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in SEQ ID NO:1 wherein the mutation is selected from R10Q, E12G, E12K, L25R, P27L, S29N, L33F, P42S, E43G, L44F, S47P, V49I, R74C, V75I, V81M, D97N, I110S, Y120C, Y120H, V123I, T124M, N133D, V138M, V139I, V144A, E154G, M158I, V1631, A172T, T173A, V174I, A177T or combinations thereof. In some embodiments, the mutated PYR/PYL receptor polypeptide further comprises at least one additional mutation at an amino acid corresponding to positions 27 and/or 63 in SEQ ID NO:1 wherein the mutation is selected from P27L, K63N, or combinations thereof. In some embodiments, the mutated PYR/PYL receptor polypeptide further comprises a mutation at an amino acid corresponding to position 119 in SEQ ID NO:1 wherein the mutation is N119Y. In some embodiments, the mutated PYR/PYL receptor polypeptide further comprises at least one mutation at an amino acid corresponding to positions 24, 82, 159, and/or 161 in SEQ ID NO:1 wherein the mutation is selected from Q24R, I82T, F159L, D161G, or combinations thereof.

In some embodiments, the method comprises growing a transgenic plant comprising at least one polynucleotide sequence encoding a mutated PYR/PYL receptor polypeptide that is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:1-119 and comprises at least one mutation at an amino acid corresponding to positions 26, 37, 71, and/or 94 in SEQ ID NO:1 wherein the mutation is selected from R37Q, F71S, E94D, or combinations thereof; whereby the transgenic plant expresses the mutated PYR/PYL receptor polypeptide.

In some embodiments, the method comprises growing a transgenic plant comprising at least one polynucleotide sequence encoding a mutated PYR/PYL receptor polypeptide that is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:1-119 and comprises at least one mutation at an amino acid corresponding to positions 110, 114, and/or 138 in SEQ ID NO:1 wherein the mutation is selected from I110T, E114D, V138M, or combinations thereof; whereby the transgenic plant expresses the mutated PYR/PYL receptor polypeptide.

In some embodiments, the method comprises growing a transgenic plant comprising at least one polynucleotide sequence encoding a mutated PYR/PYL receptor polypeptide that is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:1-119 and the mutated PYR/PYL receptor polypeptide comprises at least one mutation at an amino acid corresponding to positions 115 and/or 159 in SEQ ID NO:1 wherein the mutation is selected from H115Y, F159S, F159L, or combinations thereof; whereby the transgenic plant expresses the mutated PYR/PYL receptor polypeptide.

In some embodiments, the method comprises growing a transgenic plant comprising at least one polynucleotide sequence encoding a mutated PYR/PYL receptor polypeptide that is substantially identical to (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical to) any of SEQ ID NOs:1-119 and the mutated PYR/PYL receptor polypeptide comprises mutations at amino acid corresponding to positions 59, 120, and 158 in SEQ ID NO:1 wherein the mutations are K59R, Y120H, and M158I; whereby the transgenic plant expresses the mutated PYR/PYL receptor polypeptide. In some embodiments, the mutated PYR/PYL receptor polypeptide further comprises isoleucine residues at the amino acid positions corresponding to positions 62 and 110 in SEQ ID NO:1.

### BRIEF DESCRIPTION OF THE DRAWINGS

FIGS. 1A-1B. PYR1 mutant 27-18 confers responsiveness to fenhexamid but not ABA. Representative PYR1 mutant 27-18 (comprising S47P, K59R, and Y120H substitution mutations) was tested for responsiveness to abscisic acid (“ABA”) (FIG. 1A) and fenhexamid (FIG. 1B) at increasing concentrations as compared to a wild-type PYR1 control using an in vitro PP2C phosphatase activity assay.

FIGS. 2A-2B. Identification of residues essential for fenhexamid responsiveness in the 27C-2 mutant. FIG. 2A. The mutants (listed on left side of panel) were constructed in pBD-PYR1 and transformed into Y190 pAD-HAB1 yeast cells. The transformants were then grown on increasing concentrations of fenhexamid and scored for responsiveness using X-gal staining, performed as described in Park et al., 2009. As shown, the mutations K59R, Y120H, and M1581 are necessary for fenhexamid response by 27C-2. FIG. 2B. In vitro characterization of the PYR1 K59R, Y120H, M158I mutant. Recombinant wild-type and mutant PYR1 proteins were produced as described previously and used in PP2C assays, as described previously (Park et al., 2009), with the exception that a 2:1 ratio of receptor to phosphatase was utilized.

FIGS. 3A-3B. Engineering of fenhexamid responsiveness into PYL2. FIG. 3A. Mutations homologous to those sufficient for fenhexamid responsiveness in PYR1 (K59R, Y120H, M158I) were introduced into PYL2 (K64R, Y124H, M164I). In addition, pocket residue mutations at V114I and/or V67I were introduced. The mutants (listed on left side of panel) were constructed in pBD-PYL2 and transformed into Y190 pAD-HAB1 yeast cells. The transformants were then grown on increasing concentrations of fenhexamid and scored for responsiveness using X-gal staining, performed as described in Park et al., 2009. As shown, fenhexamid responsiveness can be engineered by the combination of these mutations. FIG. 3B. In vitro characterization of PYL2 K64R, Y124H, M164I, V67I, V114I mutant. Recombinant wild-type and mutant PYL2 proteins were produced as described previously and used in PP2C assays, as described previously (Park et al., 2009), with the exception that a 2:1 ratio of receptor for phosphatase was used.

FIG. 4. Characterization of seedling responses to fenhexamid in 35S::GFP-PYL2 K64R, Y124H, M164I, V67I, V114I transgenic plants. To investigate the sensitivity of the fenhexamid responsive PYL2 receptor in vivo, transgenic plants expressing the wild-type and mutant receptor proteins were made by agrobacterium mediated transformation. Shown are segregating GFP-expressing T2 seedlings from primary transgenic plants (T1). Columbia is the wild-type background and is non-transgenic. Two independent PYL2 K64R, Y124H, M164I, V67I, V114I (“PYL2 MUT”) transgenic lines were characterized in addition to a wild-type PYL2 overexpression line. Seeds from respective lines were germinated in the dark on media containing 100 μM fenhexamid and scored two days post-imbibition. As shown, the two transgenic lines show strong post-germination arrest while the control lines do not. The top panel shows transmitted light images of representative seedlings and the bottom panel shows epi-fluorescence confirming GFP expression of lines and showing that the expression levels between lines is similar. The data show that the PYL2 K64R, Y124H, M164I, V67I, V114I receptor protein enables activation of a well-characterized ABA response (growth inhibition) in response to fenhexamid.

FIG. 5. Characterization of seedling transcriptional responses to fenhexamid in 35S::GFP-PYL2 K64R, Y124H, M164I, V67I, V114I transgenic plants. GFP-expressing T2 seedlings from respective lines were isolated after germination on petri plates and pre-screened by epi-fluorescence microscopy and subsequently grown in liquid media culture for 7 days, after which seedlings were treated with either 100 μM fenhexamid or control (0.1% DMSO) for 6 hours. RNA was subsequently isolated from the samples and utilized in quantitative RT-PCR experiments using three well-characterized ABA responsive transcripts (as described previously, Park et al., 2009). The primer sequences used for RT-PCR analysis were obtained from Roche and synthesized by Invitrogen. Quantitative RT-PCR was performed using SybrGreen on a BioRad CFX 96 real-time PCR instrument. mRNA copy number is expressed as copy number per nanogram total RNA. Data was normalized using an ACT2 gene. Copy number was established using a standard curve of known concentration of the target genes. The data obtained show that the PYL2 K64R, Y124H, M164I, V67I, V114I receptor protein enables activation of the ABA signaling pathway in response to fenhexamid. Data shown are the average of triplicate technical replicates and error bars show standard deviation.

FIGS. 6A-6C. Fenhexamid reduces water loss in transgenic PYL2^{K64R, Y124H, M164I, V67I, V114I} expressing plants. Shown are three independent water loss experiments (FIGS. 6A-C) conducted on transgenic plants treated with control, 100 μM fenhexamid, or 100 μM ABA containing solutions. Plants were treated as described in the Examples section below and water loss from aerial rosettes was measured after detachment. In all experiments, the transgenic PYL2 line displayed reduced water loss in response to fenhexamid treatment.

FIG. 7. Fenhexamid pre-treatment does not reduce water loss in wild-type Columbia plants. Columbia plants were treated with control, 100 μM fenhexamid, or 100 μM ABA containing solutions. Plants were treated as described in the Examples section below and water loss from aerial rosettes was measured after detachment.

### DEFINITIONS

The term “PYR/PYL receptor polypeptide” refers to a protein characterized in part by the presence of one or more or all of a polyketide cyclase domain 2 (PF10604), a polyketide cyclase domain 1 (PF03364), and a Bet V I domain (PF03364), which in wild-type form mediates abscisic acid (ABA) and ABA analog signaling. A wide variety of PYR/PYL receptor polypeptide sequences are known in the art. In some embodiments, a PYR/PYL receptor polypeptide comprises a polypeptide that is substantially identical to PYR1 (SEQ ID NO:1), PYL1 (SEQ ID NO:2), PYL2 (SEQ ID NO:3), PYL3 (SEQ ID NO:4), PYL4 (SEQ ID NO:5), PYL5 (SEQ ID NO:6), PYL6 (SEQ ID NO:7), PYL7 (SEQ ID NO:8), PYL8 (SEQ ID NO:9), PYL9 (SEQ ID NO:10), PYL10 (SEQ ID NO:11), PYL11 (SEQ ID NO:12), PYL12 (SEQ ID NO:13), or PYL13 (SEQ ID NO:14), or to any of SEQ ID NOs:15-119.

A “wild-type PYR/PYL receptor polypeptide” refers to a naturally occurring PYR/PYL receptor polypeptide that mediates abscisic acid (ABA) and ABA analog signaling.

A “mutated PYR/PYL receptor polypeptide” or “modified PYR/PYL receptor polypeptide” refers to a PYR/PYL receptor polypeptide that is a variant from a naturally-occurring (i.e., wild-type) PYR/PYL receptor polypeptide. As used herein, a mutated or modified PYR/PYL receptor polypeptide comprises one or more amino acid substitutions relative to a corresponding wild-type PYR/PYL receptor polypeptide. In this context, a “mutated” polypeptide or “modified” polypeptide can be generated by any method for generating non-wild type nucleotide sequences. A mutated PYR/PYL receptor polypeptide may or may not mediate abscisic acid (ABA) and ABA analog signaling.

An amino acid “corresponding to position [X] of [specific sequence]” refers to an amino acid in a polypeptide of interest that aligns with the equivalent amino acid of a specified sequence. Generally, as described herein, the amino acid corresponding to a position of a PYR/PYL receptor polypeptide can be determined using an alignment algorithm such as BLAST. In typical embodiments of the present invention, “correspondence” of amino acid positions is determined by aligning to a region of the PYR/PYL receptor polypeptide comprising SEQ ID NO:1, as discussed further herein. When a PYR/PYL receptor polypeptide sequence differs from SEQ ID NO:1 (e.g., by changes in amino acids or addition or deletion of amino acids), it may be that a particular mutation associated with agonization by a chemical that does not agonize wild-type PYR/PYL will not be in the same position number as it is in SEQ ID NO:1. For example, amino acid position K86 of PYL1 (SEQ ID NO:2) aligns with amino acid position K59 in SEQ ID NO:1, as can be readily illustrated in an alignment of the two sequences. In this example, a mutation at amino acid position 86 in SEQ ID NO:2 corresponds to position 59 in SEQ ID NO:1.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., U.S.A.).

The phrase “substantially identical,” used in the context of two nucleic acids or polypeptides, refers to a sequence that has at least 60% sequence identity with a reference sequence. Alternatively, percent identity can be any integer from 60% to 100%. Some embodiments include at least: 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below. Embodiments of the present invention provide for nucleic acids encoding polypeptides that are substantially identical to any of SEQ ID NO:1-119.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. U.S.A. 89:10915 (1989)).

The BLAST algorithm also performs a statistical analysis of the similarity between two sequences (see, e.g., Karlin & Altschul, Proc. Nat'l. Acad. Sci. U.S.A. 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W). (see, e.g., Creighton, Proteins (1984)).

It is contemplated that a substitution mutation in a mutated PYR/PYL receptor polypeptide includes not only those specific amino acid substitutions called out in the specification, e.g. in the Examples section or in any of the Figures or Tables of the specification, but also includes amino acids that are conservative substitutions for those specific amino acids, so long as the conservatively substituted amino acid is not the wild-type amino acid. As a non-limiting example, where a mutated PYR/PYL receptor polypeptide comprises a serine-to-threonine substitution, it is contemplated that the mutated PYR/PYL receptor polypeptide may alternatively comprise a serine-to-alanine substitution, as threonine and alanine are conservative substitutions for one another; but the mutated PYR/PYL receptor polypeptide would not comprise a serine-to-serine substitution, as serine is the amino acid that is present in the wild-type PYR/PYL polypeptide.

As used herein, the term “agonist” or “agonists” refers to a molecule identified using in vitro and in vivo assays for activity of a described target protein as described elsewhere herein. Agonists are agents that, e.g., induce or activate the expression of a described target protein or bind to, stimulate, increase, open, activate, facilitate, enhance activation, sensitize or up-regulate the activity of described target protein (or encoding polynucleotide). Agonists include naturally occurring and synthetic molecules. In some embodiments, the agonists are agrichemicals, e.g., fungicides, herbicides, pesticides, and/or fertilizers. Assays for determining whether an agonist “agonizes” or “does not agonize” a target protein include, e.g., contacting putative agonists to purified target protein(s) and then determining the functional effects on the described target protein activity, as described above, or contacting putative agonists to cells expressing the target protein(s) and then determining the functional effects on the described target protein activity, as described above. One of skill in the art will be able to determine whether an assay is suitable for determining whether an agonist agonizes or does not agonize a target protein. Samples or assays comprising described target protein that are treated with a putative agonist are compared to control samples without the agonist to examine the extent of effect. Control samples (untreated with agonists) are assigned a relative activity value of 100%. Agonism of the described target protein is achieved when the activity value relative to the control is 110%, optionally 150%, optionally 200%, 300%, 400%, 500%, or 1000-3000% or more higher.

As used herein, the term “orthogonal receptor” refers to a receptor that has been modified to selectively recognize new ligands (“orthogonal ligands”). As used herein, the term “orthogonal ligand” refers to an agent that agonizes a mutated or modified PYR/PYL receptor polypeptide but which does not agonize a wild-type PYR/PYL receptor polypeptide. In some embodiments, the orthogonal ligands are agrichemicals, e.g., fungicides, herbicides, pesticides, nematicides, plant activators, synergists, herbicide safeners, plant growth regulators, insect repellants, and/or fertilizers.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct that, when introduced into a host cell, results in transcription and/or translation of an RNA or polypeptide, respectively.

Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence.

As used herein, the terms “abiotic stress,” “stress,” or “stress condition” refer to the exposure of a plant, plant cell, or the like, to a non-living (“abiotic”) physical or chemical agent that has an adverse effect on metabolism, growth, development, propagation, or survival of the plant (collectively, “growth”). A stress can be imposed on a plant due, for example, to an environmental factor such as water (e.g., flooding, drought, or dehydration), anaerobic conditions (e.g., a lower level of oxygen or high level of CO₂), abnormal osmotic conditions, salinity, or temperature (e.g., hot/heat, cold, freezing, or frost), a deficiency of nutrients or exposure to pollutants, or by a hormone, second messenger, or other molecule. Anaerobic stress, for example, is due to a reduction in oxygen levels (hypoxia or anoxia) sufficient to produce a stress response. A flooding stress can be due to prolonged or transient immersion of a plant, plant part, tissue, or isolated cell in a liquid medium such as occurs during monsoon, wet season, flash flooding, or excessive irrigation of plants, or the like. A cold stress or heat stress can occur due to a decrease or increase, respectively, in the temperature from the optimum range of growth temperatures for a particular plant species. Such optimum growth temperature ranges are readily determined or known to those skilled in the art. Dehydration stress can be induced by the loss of water, reduced turgor, or reduced water content of a cell, tissue, organ or whole plant. Drought stress can be induced by or associated with the deprivation of water or reduced supply of water to a cell, tissue, organ or organism. Salinity-induced stress (salt-stress) can be associated with or induced by a perturbation in the osmotic potential of the intracellular or extracellular environment of a cell. As used herein, the term “abiotic stress tolerance” or “stress tolerance” refers to a plant's increased resistance or tolerance to abiotic stress as compared to plants under normal conditions and the ability to perform in a relatively superior manner when under abiotic stress conditions. As used herein, the terms “drought resistance” and “drought tolerance” are used to refer to a plant's increased resistance or tolerance to stress induced by a reduction in water availability, as compared to normal circumstances, and the ability of the plant to function and survive in lower-water environments, and perform in a relatively superior manner.

### DETAILED DESCRIPTION OF THE INVENTION

### I. Introduction

Surprisingly, proteins belonging to a family of abscisic acid (ABA) receptors, the PYR/PYL receptor family, can be mutated to bind and respond to chemicals other than ABA. It was found that certain agrochemicals, when contacted to wild-type PYR/PYL receptor polypeptides in the presence of ABA, lowered the level of PYR/PYL receptor activation by ABA. An additional surprising revelation was the discovery that the amino acid corresponding to residue K59 of PYR1, a conserved residue of the PYR/PYL ligand-binding pocket that contacts ABA in wild-type PYR/PYL receptors, can be mutated to many variants to enable the creation of orthogonal receptors for multiple orthogonal ligands.

When PYR/PYL receptor polypeptides were subsequently mutagenized and screened to establish whether PYR/PYL receptor binding to these new chemical agonists could be improved, it was unexpectedly discovered that certain mutations in PYR/PYL receptor polypeptides resulted in activation of the mutated PYR/PYL polypeptide by the non-natural ligands (orthogonal ligands). Moreover, in some cases, the mutations restructured the ligand-binding pockets of PYR/PYL and thus simultaneously abolished the ability of the natural ligand (ABA) to activate the mutated PYR/PYL polypeptide.

Thus, it is possible to alter ABA receptors such as PYR/PYL receptor polypeptides so that a compound other than ABA can be used to selectively activate them. Moreover, because the mutated PYR/PYL receptor (orthogonal receptor) can be selectively activated by applying an orthogonal ligand (e.g., as part of a program to improve plant response to water deficit), the problem of “yield drag” can be avoided. Yield drag is traditionally associated with receptor over-expression, in which gene over-expression during normal or optimal growth conditions (i.e., in the absence of drought or other stressors) is associated with slowed growth.

### II. Mutated PYR/PYL Receptor Polypeptides

The present invention provides for mutated PYR/PYL receptor polypeptides that are agonized by chemicals that do not agonize wild-type PYR/PYL receptor polypeptides, as well as polynucleotides encoding mutated PYR/PYL receptor polypeptides that are agonized by chemicals that do not agonize wild-type PYR/PYL receptor polypeptides; expression cassettes and expression vectors comprising polynucleotides encoding mutated PYR/PYL receptor polypeptides that are agonized by chemicals that do not agonize wild-type PYR/PYL receptor polypeptides; plants comprising mutated PYR/PYL receptor polypeptides that are agonized by chemicals that do not agonize wild-type PYR/PYL receptor polypeptides; methods of making plants comprising mutated PYR/PYL receptor polypeptides that are agonized by chemicals that do not agonize wild-type PYR/PYL receptor polypeptides; and methods of making mutated PYR/PYL receptor polypeptides.

In some embodiments, the mutated PYR/PYL receptor polypeptide, but not the wild-type PYR/PYL receptor polypeptide, is agonized by the chemical bromoxynil, chloroxynil, ioxynil, coumatetralyl, dichlobenil, fenhexamid, benoxacor, or BTH (acibenzolar-s-methyl) when the chemical is contacted to the PYR/PYL receptor polypeptide. In another embodiment of the present invention, mutated PYR/PYL receptor polypeptides are agonized by chemicals that do not agonize wild-type PYR/PYL receptor polypeptides and also are agonized by ABA, a compound that does agonize wild-type PYR/PYL receptor polypeptides.

A wide variety of wild-type (naturally occurring) PYR/PYL polypeptide sequences are known in the art. Although PYR1 was originally identified as an abscisic acid (ABA) receptor in Arabidopsis, in fact PYR1 is a member of a group of at least 14 proteins (PYR/PYL proteins) in the same protein family in Arabidopsis that also mediate ABA signaling. This protein family is also present in other plants (see, e.g., SEQUENCE LISTING) and is characterized in part by the presence of one or more or all of a polyketide cyclase domain 2 (PF10604), a polyketide cyclase domain 1 (PF03364), and a Bet V I domain (PF03364). START/Bet v 1 superfamily domain are described in, for example, Radauer, BMC Evol. Biol. 8:286 (2008). In some embodiments, a wild-type PYR/PYL receptor polypeptide comprises any of SEQ ID NOs:1-119. In some embodiments, a wild-type PYR/PYL receptor polypeptide is substantially identical to (e.g., at least 70%, 75%, 80%, 85%, 90%, 91%, 92%, 93%, 94% 95%, 96%, 97%, 98%, or 99% identical to) any of SEQ ID NOs:1-119.

Mutated PYR/PYL receptor polypeptides are variants from naturally-occurring (i.e., wild-type) PYR/PYL receptor polypeptides. Variants include, e.g., fusion proteins, deletions, insertions, or mutations that retain activity. In some embodiments, a mutated PYR/PYL receptor polypeptide is substantially identical to (e.g., at least 70%, 75%, 80%, 85%, 90%, 91%, 92%, 93%, 94% 95%, 96%, 97%, 98%, or 99% identical to) any of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, or 119 and comprises 1, 2, 3, 4, 5, 6, or more mutations as described herein relative to a corresponding wild-type PYR/PYL receptor polypeptide. In addition, in some embodiments, the mutated PYR/PYL receptor polypeptide further comprises an amino- and/or carboxyl terminal fusion with a heterologous amino acid sequence.

The inventors have found a number of mutations that affect response to chemicals. Some mutations occur across different chemicals tested and appear to allow in some cases for the modified PYR/PYL receptor protein to function in response to a diverse array of chemicals. This is not always the case; some mutations appear to be selective for one chemical and do not appear to promote activation of the modified PYR/PYL receptor protein by other chemicals. In some cases, a single mutation is sufficient for a modified PYR/PYL receptor protein to be agonized by a chemical agonist. In other cases, multiple mutations allow a modified PYR/PYL receptor protein to be agonized by a chemical agonist. In some cases, the modified PYR/PYL receptor protein contains two, three, four, five, six, or more mutations in order for the protein to be agonized by a chemical agonist.

The modified PYR/PYL receptor proteins may be mutated at any location in the PYR/PYL receptor polypeptide sequence. In some cases, and as discussed herein, the mutation may occur in the ligand-binding pocket of the PYR/PYL polypeptide. However, the mutation need not occur in the ligand-binding pocket of the PYR/PYL polypeptide in order to effectuate activation by an orthogonal ligand. Without being bound to a particular theory, it is hypothesized that mutations outside the ligand-binding pocket that result in activation of a PYR/PYL receptor protein by an orthogonal ligand have an indirect effect on ligand-binding architecture that results in the ability of the receptor to be activated by the orthogonal ligand. It is further hypothesized that specific mutations at the position corresponding to amino acid K59 in PYR1 (SEQ ID NO:1) enable PYR/PYL receptor polypeptides to be more easily activated by orthogonal ligands than wild-type PYR/PYL receptor polypeptides, which is a desirable property for engineering orthogonal receptors.

Any of the mutations described herein can be made in the polypeptides of any of SEQ ID NOs:1-119 or in polypeptides substantially identical to any of SEQ ID NOs:1-119. Alternatively, any of the mutations described above can be made in a polypeptide comprising any of the consensus sequences that identify PYR/PYL proteins, for example as set forth below.

### Consensus Sequences

PYR/PYL receptor proteins can be described by reference to sequence alignments that identify conserved amino acid or motifs (i.e., where alteration in sequences may alter protein function) and regions where variation occurs in alignment of sequences (i.e., where variation of sequence is not likely to significantly affect protein activity). SEQ ID NOs:120-123 provide consensus sequences useful for identifying wild-type PYR/PYL receptor polypeptides. The consensus sequences of SEQ ID NOs:120-123 were generated by aligning all 14 members of the Arabidopsis PYR/PYL receptor protein family. In the consensus sequences of SEQ ID NOs:120-123, the capitalized letter represents an amino acid residue that is absolutely conserved among all 14 members of the Arabidopsis PYR/PYL receptor protein family, while “x” represents an amino acid residue that is not absolutely conserved among all 14 family members and which can be any amino acid. It will be appreciated that when selecting an amino acid to insert at a position marked by an “x” that in some embodiments, the amino acid is selected from those amino acids found at the corresponding position in a wild-type or mutated PYR/PYL protein.

### PYR1 to PYL13

(SEQ ID NO: 120)
CxSxxxxxxxAPxxxxWxxxxxFxxPxxxxxFxxxC
(SEQ ID NO: 121)
GxxRxVxxxSxxPAxxSxExLxxxD
(SEQ ID NO: 122)
GGxHRLxNYxS
(SEQ ID NO: 123)
ESxxVDxPxGxxxxxTxxFxxxxxxxNLxxL

Consensus sequence CxSxxxxxxxAPxxxxWxxxxxFxxPxxxxxFxxxC (SEQ ID NO:120) comprises the region corresponding to amino acids 30 to 65 of PYR1 (SEQ ID NO:1). Consensus sequence GxxRxVxxxSxxPAxxSxExLxxxD (SEQ ID NO:121) comprises the region corresponding to amino acids 76 to 100 of PYR1 (SEQ ID NO:1). Consensus sequence GGxHRLxNYxS (SEQ ID NO:122) comprises the region corresponding to amino acids 112 to 122 of PYR1 (SEQ ID NO:1). ESxxVDxPxGxxxxxTxxFxxxxxxxNLxxL (SEQ ID NO:123) comprises the region corresponding to amino acids 141 to 171 of PYR1 (SEQ ID NO:1).

In some cases, a PYR/PYL mutation occurs at a residue within a consensus sequence of SEQ ID NOs:120-123. In some cases, the mutation occurs at a residue that is absolutely conserved among all 14 members of the PYR/PYL receptor protein family. In some cases, the mutation occurs at a residue that is not absolutely conserved. As described herein, a substitution mutation at a residue within a consensus sequence is depicted as a bracket in place of an amino acid of the consensus sequence. Where more than one amino acid is enclosed by said bracket, it indicates that any of the amino acids enclosed by said bracket may be substituted for the wild-type amino acid at that residue of the consensus sequence.

Additionally, the modified PYR/PYL receptor protein may comprise more than one mutation within a consensus sequence, or may comprise at least two or more consensus sequences with each consensus sequence having at least one mutation.

K59. In some cases, the modified PYR/PYL receptor comprises the consensus sequence CxSxxxxxxxAPxxxxWxxxxxFxxPxxx[ACDEFGHLMNQRSTVYW]xFxxxC (SEQ ID NO:149).

Y120. In some cases, the modified PYR/PYL receptor comprises the consensus sequence GGxHRLxN[HC]xS (SEQ ID NO:150).

I110. In some cases, the modified PYR/PYL receptor comprises the consensus sequence [STCAYW]xGGxHRLxNYxS (SEQ ID NO:151).

P42. In some cases, the modified PYR/PYL receptor comprises the consensus sequence CxSxxxxxxxAP[ST]xxxWxxxxxFxxPxxxxxFxxxC (SEQ ID NO:152).

S47. In some cases, the modified PYR/PYL receptor comprises the consensus sequence CxSxxxxxxxAPxxxxW[PRA]xxxxFxxPxxxxxFxxxC (SEQ ID NO:153).

K59 and Y120. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and GGxHRLxN[HC]Xs (SEQ ID NO:150).

K59 and I110. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and [STCAYW]xGGxHRLxNYxS (SEQ ID NO:151).

K59 and P42. In some cases, the modified PYR/PYL receptor comprises the consensus sequence CxSxxxxxxxAP[ST]xxxWxxxxxFxxPxxx[ACDEFGHLMNQRSTVYW]xFxxxC (SEQ ID NO:154).

K59 and S47. In some cases, the modified PYR/PYL receptor comprises the consensus sequence CxSxxxxxxxAPxxxxW[PRA]xxxxFxxPxxx[ACDEFGHLMNQRSTVYW]xFxxxC (SEQ ID NO:155).

H60. In some cases, the modified PYR/PYL receptor comprises the consensus sequence CxSxxxxxxxAPxxxxWxxxxxFxxPxxxx[R]FxxxC (SEQ ID NO:156).

S92. In some cases, the modified PYR/PYL receptor comprises the consensus sequence GxxRxVxxxSxxPAxx[T]xExLxxxD (SEQ ID NO:157).

E140. In some cases, the modified PYR/PYL receptor comprises the consensus sequence [GQD]SxxVDxPxGNxxxxTxxFxxxxxxxNLxxL (SEQ ID NO:158).

K59 and H60. In some cases, the modified PYR/PYL receptor comprises the consensus sequence CxSxxxxxxxAPxxxxWxxxxxFxxPxxx[ACDEFGHLMNQRSTVYW][R]FxxxC (SEQ ID NO:159).

K59 and S92. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and GxxRxVxxxSxxPAxx[T]xExLxxxD (SEQ ID NO:157).

K59 and E140. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and [GQD]SxxVDxPxGNxxxxTxxFxxxxxxxNLxxL (SEQ ID NO:158).

E94. In some cases, the modified PYR/PYL receptor comprises the consensus sequence GxxRxVxxxSxxPAxxSx[D]xLxxxD (SEQ ID NO:160).

K59 and E94. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and GxxRxVxxxSxxPAxxSx[D]xLxxxD (SEQ ID NO:160).

N119. In some cases, the modified PYR/PYL receptor comprises the consensus sequence GGxHRLx[Y]YxS (SEQ ID NO:161).

K59 and N119. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and GGxHRLx[Y]YxS (SEQ ID NO:161).

H115. In some cases, the modified PYR/PYL receptor comprises the consensus sequence GGx[Y]RLxNYxS (SEQ ID NO:162).

F159. In some cases, the modified PYR/PYL receptor comprises the consensus sequence ESxxVDxPxGNxxxxTxx[SL]xxxxxxxNLxxL (SEQ ID NO:163).

K59 and F159. In some cases, the modified PYR/PYL receptor comprises the consensus sequences CxSxxxxxxxAPxxxxWxxxxxFxxPxxx[ACDEFGHLMNQRSTVYW]xFxxxC (SEQ ID NO:149) and ESxxVDxPxGNxxxxTxx[SL]xxxxxxxNLxxL (SEQ ID NO:163).

Accordingly, in some embodiments, the mutated PYR/PYL receptor polypeptides of the invention comprise one or more of the above-described consensus sequences (SEQ ID NOs:149-163) or conservative variants thereof. In some embodiments, the present invention provides for polynucleotides encoding one or more mutated PYR/PYL receptor polypeptides comprising one or more of the above-described consensus sequences (SEQ ID NOs:149-163) or conservative variants thereof.

Modified PYR/PYL receptor proteins can alternatively be described by reference to sequence identifiers (“SEQ IDs”) for PYR/PYL polypeptides. Modified PYR/PYL receptors may comprise a SEQ ID listed herein and further comprise at least one (e.g., 1, 2, 3, 4, 5, 6, 7, 8, 9, 10, etc.) mutation at an amino acid position (e.g., a substitution, deletion, or insertion mutation) as described herein. Thus, in some embodiments, a mutated PYR/PYL receptor polypeptide comprises any of SEQ ID NOs:1-119 and further comprises at least one mutation at any amino acid position.

Alternatively, modified PYR/PYL receptors may be substantially identical to a SEQ ID listed herein and further comprise at least one (e.g., 1, 2, 3, 4, 5, 6, 7, 8, 9, 10, etc.) mutation at an amino acid position. In some embodiments, a mutated PYR/PYL receptor polypeptide is substantially identical to any of SEQ ID NOs:1-119 and further comprises at least one mutation at any amino acid position.

In some instances, the modified PYR/PYL receptor protein has been modified at the residue corresponding to amino acid position 59 in PYR1 (SEQ ID NO:1). A mutation at this residue, also called “K59,” disrupts ABA responsiveness in the modified PYR/PYL receptor protein.

In some cases, a K59 mutation is not only sufficient to abolish ABA sensitivity in the modified PYR/PYL receptor protein, it is also sufficient to confer sensitivity to a new chemical agonist (“orthogonal ligand”) on a modified PYR/PYL receptor protein. In some cases, the K59 mutation that results in receptor activation by an orthogonal ligand is a substitution of an alanine residue for the wild-type residue, a substitution of a cysteine residue for the wild-type residue, a substitution of an aspartic acid residue for the wild-type residue, a substitution of a glutamic acid residue for the wild-type residue, a substitution of a phenylalanine residue for the wild-type residue, a substitution of a glycine residue for the wild-type residue, a substitution of a histidine residue for the wild-type residue, a substitution of a leucine residue for the wild-type residue, a substitution of a methionine residue for the wild-type residue, a substitution of an asparagine residue for the wild-type residue, a substitution of a glutamine residue for the wild-type residue, a substitution of an arginine residue for the wild-type residue, a substitution of a serine residue for the wild-type residue, a substitution of a threonine residue for the wild-type residue, a substitution of a valine residue for the wild-type residue, a substitution of a tyrosine residue for the wild-type residue, or a substitution of a tryptophan residue for the wild-type residue.

Additionally, the modified PYR/PYL receptor protein may comprise at least one more mutation in addition to the K59 mutation. It was found that numerous chemical agonists activate modified PYR/PYL receptor proteins when the modifications include a K59 mutation and one, two, three, four, or five additional mutations at other residues in the protein, as shown below in Tables 1-5. A non-limiting list of exemplary combinations of mutations, for which K59 is one mutation site, and for which the modified PYR/PYL receptor is not agonized by ABA but is agonized by an orthogonal ligand, includes:
547, K59, and Y120
K59, Y120, and V144
P42, K59, and Y120
P42, K59, Y120, and T124
P42, K59, Y120, and E154
E12, E43, K59, I110, and N133
E12, L25, E43, K59, I110, and N133
P27, K59, and K63
T57 and K59
R50, K59, and E141
K59, H60, and N151
K59, H60, E102, T125, and E141
K59 and 182
K59 and S92
H21, K59, H60, S92, and R116
P41, K59, and H60
K59 and N119
Q24, K59, I82, F159, and D161
K59, Y120, and M158
P42, K59, Y120, and M158
P42, K59, D97,Y120, V163, and A172
P42, L44, K59, Y120, V138, and M158
P42, K59, Y120, V123, V139, and M158
S47, V49, K59, Y120, M158, and A177
K59, V81, Y120, M158, and V163
P27, P42, K59, D97, Y120, M158, and T173
P42, K59, R74, Y120, and M158
S29, K59, D97, Y120, V163, and A172
P42, K59, Y120, V123, V139, M158, and V163
K59, V81, Y120, M158, and V163
E12, K59, V75, D97, Y120, V163, and A172
L33, P42, K59, Y120, V123, and M158
P42, K59, Y120, M158, V163, and V174
R10, P42, K59, D97, Y120, V163, and A172

Thus, in some embodiments, the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, asparagine, glutamine, arginine, serine, threonine, valine, tyrosine, or tryptophan.

In some embodiments, the mutated PYR/PYL receptor polypeptide further comprises at least one additional mutation from a naturally-occurring residue to a non-naturally-occurring residue at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in PYR1 (SEQ ID NO:1). In some embodiments, the mutated PYR/PYL receptor polypeptide comprises at least one additional mutation from a naturally-occurring residue at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in PYR1 (SEQ ID NO:1) to an alanine residue, a cysteine residue, an aspartic acid residue, a glutamic acid residue, a phenylalanine residue, a glycine residue, a histidine residue, an isoleucine residue, a lysine residue, a leucine residue, a methionine residue, an asparagine residue, a proline residue, a glutamine residue, an arginine residue, a serine residue, a threonine residue, a valine residue, a tryptophan residue, or a tyrosine residue. In some embodiments, the mutated PYR/PYL receptor polypeptide comprises at least one additional mutation at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in PYR1 (SEQ ID NO:1) wherein the mutation is selected from R10Q, E12G, E12K, L25R, P27L, S29N, L33F, P42S, E43G, L44F, S47P, V49I, R74C, V75I, V81M, D97N, I110S, Y120C, Y120H, V1231, T124M, N133D, V138M, V139I, V144A, E154G, M158I, V163I, A172T, T173A, V174I, A177T or combinations thereof. In some embodiments, the mutated PYR/PYL receptor polypeptide still further comprises an isoleucine residue at one or more of the amino acid positions corresponding to positions 62 and 110 in PYR1 (SEQ ID NO:1). In some embodiments, the mutated PYR/PYL receptor polypeptide comprises a valine-to-isoleucine mutation at one or more of the amino acid positions corresponding to positions 62 and 110 in PYR1 (SEQ ID NO:1). In some embodiments, the present invention provides for a polynucleotide encoding one or more of said mutated PYR/PYL receptor polypeptides.

In some instances, the modified PYR/PYL receptor protein has been modified at a residue other than the K59 residue. A non-limiting exemplary list of mutations other than K59 which abolish ABA agonization but promote orthogonal ligand activation of a modified PYR/PYL receptor protein includes:
D26
E94
F159

Thus, in some embodiments, the mutated PYR/PYL receptor polypeptide comprises a mutation at an amino acid corresponding to amino acid position 26 in PYR1 (SEQ ID NO:1) wherein the mutation is D26G. In some embodiments, the mutated PYR/PYL receptor polypeptide comprises a mutation at an amino acid corresponding to amino acid position 94 in PYR1 (SEQ ID NO:1) wherein the mutation is E94D. In some embodiments, the mutated PYR/PYL receptor polypeptide comprises a mutation at an amino acid corresponding to amino acid position 159 in PYR1 (SEQ ID NO:1) wherein the mutation is F159L. In some embodiments, the present invention provides for a polynucleotide encoding one or more of said mutated PYR/PYL receptor polypeptides.

In some instances, the modified PYR/PYL receptor protein has been modified at least two residues other than the K59 residue. A non-limiting list of exemplary combinations of mutations, which do not include a K59 mutation, and which abolish ABA agonization but promote orthogonal ligand activation of a modified PYR/PYL receptor protein, includes:
R37 and F71
I110, E114, and V138
H115 and F159

Thus, in some embodiments, the mutated PYR/PYL receptor polypeptide comprises at least two mutations at amino acids corresponding to positions 37, 71, 110, 114, 115, 138, and/or 159 in PYR1 (SEQ ID NO:1) wherein the mutations are selected from R37Q, F71S, I110T, E114D, H115Y, V138M, F159S, or combinations thereof. In some embodiments, the present invention provides for a polynucleotide encoding one or more of said mutated PYR/PYL receptor polypeptides.

In some embodiments, a mutated PYR/PYL receptor polypeptide comprises any of SEQ ID NOs:124-148 or 164-178.

### Ligand-Binding Pocket of PYR/PYL

PYR/PYL receptor proteins have a conserved START-domain ligand-binding pocket flanked by two loops called the “gate” and the “latch” (Melcher, K. et al., Nature 462 (2009)). ABA binds to a PYR/PYL receptor protein at the ligand-binding pocket and ABA binding induces closure of the loops to seal ABA inside the ligand-binding pocket. In wild-type PYR/PYL receptor proteins, residues comprising the ligand-binding pocket are those residues with side chains that are within 4 angstroms of ABA or the water molecules that accompany ABA when ABA binds in the pocket of the PYR/PYL receptor protein. For example, the residues comprising the ligand-binding pocket of PYR1 (SEQ ID NO:1) are: P55, K59, F61, I62, R79, V81, V83, P88, A89, S92, E94, F108, I110, H115, L117, Y120, E141, F159, V163, and N167.

In some embodiments, the PYR/PYL receptor polypeptides are mutated at least one amino acid residue comprising the ligand-binding pocket of the PYR/PYL receptor protein. Accordingly, in some embodiments the mutated PYR/PYL receptor polypeptide comprises at least one mutation at an amino acid corresponding to positions 55, 59, 61, 62, 79, 81, 83, 88, 89, 92, 94, 108, 110, 115, 117, 120, 141, 159, 163, and/or 167 in PYR1 (SEQ ID NO:1) wherein the mutation is selected from P55, K59, F61, I62, R79, V81, V83, P88, A89, S92, E94, F108, I110, H115, L117, Y120, E141, F159, V163, N167, or combinations thereof. In some embodiments, the present invention provides for a polynucleotide encoding one or more of said mutated PYR/PYL receptor polypeptides.

Embodiments of the present invention provide for use of the above proteins and/or nucleic acid sequences, encoding such polypeptides, in the methods and compositions (e.g., expression cassettes, plants, etc.) of the present invention. The isolation of a polynucleotide sequence encoding a plant wild-type PYR/PYL receptor (e.g., from plants where PYR/PYL sequences have not yet been identified) may be accomplished by a number of techniques. For instance, oligonucleotide probes based on the PYR/PYL coding sequences disclosed (e.g., as listed in the SEQUENCE LISTING) here can be used to identify the desired wild-type PYR/PYL gene in a cDNA or genomic DNA library. To construct genomic libraries, large segments of genomic DNA are generated by random fragmentation, e.g., using restriction endonucleases, and are ligated with vector DNA to form concatemers that can be packaged into the appropriate vector. To prepare a cDNA library, mRNA is isolated from the desired tissue, such as a leaf from a particular plant species, and a cDNA library containing the gene transcript of interest is prepared from the mRNA. Alternatively, cDNA may be prepared from mRNA extracted from other tissues in which PYR/PYL gene is expressed.

The cDNA or genomic library can then be screened using a probe based upon the sequence of a PYR/PYL gene disclosed here. Probes may be used to hybridize with genomic DNA or cDNA sequences to isolate homologous genes in the same or different plant species. Alternatively, antibodies raised against a polypeptide can be used to screen an mRNA expression library.

Alternatively, the nucleic acids encoding PYR/PYL can be amplified from nucleic acid samples using amplification techniques. For instance, polymerase chain reaction (PCR) technology can be used to amplify the coding sequences of PYR/PYL directly from genomic DNA, from cDNA, from genomic libraries or cDNA libraries. PCR and other in vitro amplification methods may also be useful, for example, to clone polynucleotide sequences encoding PYR/PYL to be expressed, to make nucleic acids to use as probes for detecting the presence of the desired mRNA in samples, for nucleic acid sequencing, or for other purposes. For a general overview of PCR see PCR Protocols: A Guide to Methods and Applications. (Innis, M., Gelfand, D., Sninsky, J. and White, T., eds.), Academic Press, San Diego (1990). Appropriate primers and probes for identifying sequences from plant tissues are generated from comparisons of the sequences provided here with other related genes.

In some embodiments, the partial or entire genome of a number of plants has been sequenced and open reading frames identified. By a BLAST search, one can identify the coding sequence for wild-type PYR/PYL in various plants.

### III. Chemical Agonists and Agonist Formulations

Embodiments of the present invention provide for agricultural chemical formulations formulated for contacting to mutated PYR/PYL receptor polypeptides and/or plants comprising mutated PYR/PYL receptor polypeptides, wherein the formulation comprises an agonist of a mutated PYR/PYL polypeptide of the present invention. In some embodiments, the agricultural chemical agonist comprises a fungicide, an herbicide, a pesticide, a nematicide, a plant activator, a synergist, an herbicide safener, a plant growth regulator, an insect repellant, or a fertilizer. In some embodiments, the agricultural chemical agonist is selected from the group consisting of bromoxynil, chloroxynil, ioxynil, coumatetralyl, dichlobenil, fenhexamid, benoxacor, and BTH (acibenzolar-s-methyl).

Agrochemicals are often prepared and applied to plants as esters or salts, which may improve uptake and efficacy. The action of ubiquitous cellular esterases can convert esters (or homologous compounds such as the S-methyl derivatives of acibenzolar) into free acids or alcohols, which are the bioactive forms. For example, bromoxynil butyrate, bromoxynil heptanoate, bromoxynil octanoate, and bromoxynil-potassium are alternate formulations of bromoxynil. After absorption these compounds form the same bioactive species (bromoxynil) in plants. Similar variants of chloroxynil and ioxynil can be made and are known (for example, ioxynil-lithium, ioxynil octanoate, ioxynil-sodium, etc.). BTH is an S-methyl derivative of acibenzolar, the active form of BTH in plants.

### Fenhexamid

It was found that mutating the amino acid corresponding to K59 in SEQ ID NO:1, along with mutating at least one more amino acid in the PYR/PYL receptor polypeptide, resulted in activation of the modified receptor by fenhexamid (Tables 1, 7, and 8). A non-limiting list of exemplary combinations of mutations that result in modified PYR/PYL receptor being agonized by fenhexamid includes:
S47P, K59R, and Y120H
K59R, Y120H, and V144A
P42S, K59R, and Y120C
P42S, K59R, Y120C, and T124M
P42S, K59R, Y120C, and E154G
E12G, E43G, K59R, I110S, and N133D
E12G, L25R, E43G, K59R, I110S, and N133D
K59R, Y120H, and M158I
P42S, K59R, Y120H, and M158I
P42S, K59R, D97N, Y120H, V163I, and A172T
P42S, L44F, K59R, Y120H, V138M, and M158I
P42S, K59R, Y120H, V123I, V139I, and M158I
S47P, V49I, K59R, Y120H, M158I, and A177T
K59R, V81M, Y120C, M158I, and V163I
P27L, P42S, K59R, D97N, Y120H, M158I, and T173A
P42S, K59R, R74C, Y120H, and M158I
S29N, K59R, D97N, Y120H, V163I, and A172T
P42S, K59R, Y120H, V123I, V139I, M158I, and V163I
K59R, V81M, Y120H, M158I, and V163I
E12K, K59R, V75I, D97N, Y120H, V163I, and A172T
L33F, P42S, K59R, Y120H, V123I, and M158I
P42S, K59R, Y120H, M158I, V163I, and V174I
R10Q, P42S, K59R, D97N, Y120H, V163I, and A172T

Thus, in some embodiments a modified PYR/PYL receptor is agonized by fenhexamid. In some embodiments, the modified PYR/PYL protein comprises one or more sets of mutations corresponding to those set forth above. In some embodiments, the modified PYR/PYL receptor protein comprising any of the sets of mutations corresponding to those set forth above further comprises an isoleucine residue at one or more of the amino acid positions corresponding to positions 62 and 110 in PYR1 (SEQ ID NO:1). In some embodiments, the modified PYR/PYL receptor protein comprises a valine-to-isoleucine mutation at one or more the amino acid positions corresponding to positions 62 and 110 in PYR1 (SEQ ID NO:1) (e.g., valine-to-isoleucine mutations at amino acid positions 64 and/or 114 in PYL2 (SEQ ID NO:3), which correspond to positions 62 and 110, respectively, in PYR1). In some embodiments, the modified PYR/PYL receptor proteins are substantially identical to any of SEQ ID NOs:1-119 and/or comprise one or more consensus sequence selected from SEQ ID NOs:149-163. In some embodiments, the present invention provides for a polynucleotide encoding one or more of said modified PYR/PYL receptor polypeptides.

Additionally, in some embodiments of the present invention, a method of screening for modified PYR/PYL receptor polypeptides includes screening the modified polypeptide to determine whether it is agonized by fenhexamid.

### Bromoxynil

It was also found that mutating the amino acid corresponding to K59 in SEQ ID NO:1, along with mutating at least one more amino acid in the PYR/PYL receptor polypeptide, resulted in activation of the modified receptor by bromoxynil (Table 2). A non-limiting list of exemplary combinations of mutations that result in modified PYR/PYL receptor being agonized by bromoxynil includes:
T57A and K59R
R50G, K59R, and E141Q
K59, H60R, and N151D
K59R, H60R, E102G, T125A, and E141D
K59R and I82N
K59R and S92T
H21Y, K59R, H60R, S92T, and R116K
P41L, K59R, and H60R

Thus, in some embodiments a modified PYR/PYL receptor of the present invention is agonized by bromoxynil or the bromoxynil analogs chloroxynil and ioxynil. In some embodiments, the modified PYR/PYL protein comprises one or more sets of mutations corresponding to those set forth above. In some embodiments, such proteins are substantially identical to any of SEQ ID NOs:1-119 and/or comprise one or more consensus sequence selected from SEQ ID NOs:149-163. In some embodiments, the present invention provides for a polynucleotide encoding one or more of said modified PYR/PYL receptor polypeptides.

Additionally, in some embodiments of the present invention, a method of screening for modified PYR/PYL receptor polypeptides includes screening the modified polypeptide to determine whether it is agonized by bromoxynil or the bromoxynil analogs chloroxynil and ioxynil.

### Dichlobenil

In some cases, mutating the amino acid corresponding to K59 in SEQ ID NO:1 alone results in activation of the modified receptor by dichlobenil (Table 3). In some cases, mutating the amino acid corresponding to K59 in SEQ ID NO:1, along with mutating at least one more amino acid in the PYR/PYL receptor polypeptide, results in activation of the modified receptor by dichlobenil. In some cases, the amino acid corresponding to K59 in SEQ ID NO:1 does not need be mutated in the modified PYR/PYL receptor polypeptide in order for dichlobenil to activate the receptor, so long as there is at least one mutation in another amino acid in the PYR/PYL receptor polypeptide. A non-limiting list of exemplary mutations and combinations of mutations that result in modified PYR/PYL receptor being agonized by dichlobenil includes:
K59R
D26G
E94D
R37Q and F71S
P27L, K59R, and K63N

Thus, in some embodiments a modified PYR/PYL receptor is agonized by dichlobenil. In some embodiments, the modified PYR/PYL protein comprises one or more sets of mutations corresponding to those set forth above. In some embodiments, such proteins are substantially identical to any of SEQ ID NOs:1-119 and/or comprise one or more consensus sequence selected from SEQ ID NOs:149-163. In some embodiments, the present invention provides for a polynucleotide encoding one or more of said modified PYR/PYL receptor polypeptides.

Additionally, in some embodiments of the present invention, a method of screening for modified PYR/PYL receptor polypeptides includes screening the modified polypeptide to determine whether it is agonized by dichlobenil.

### Benoxacor

In some cases, mutating the amino acid corresponding to K59 in SEQ ID NO:1 alone results in activation of the modified receptor by benoxacor (Table 4). In some cases, the amino acid corresponding to K59 in SEQ ID NO:1 does not need be mutated in the modified PYR/PYL receptor polypeptide in order for benoxacor to activate the receptor, so long as there is at least one mutation in another amino acid in the PYR/PYL receptor polypeptide. A non-limiting list of exemplary combinations of mutations that result in modified PYR/PYL receptor being agonized by benoxacor includes:
K59R and N119Y
I110T, E114D, and V138M

Thus, in some embodiments a modified PYR/PYL receptor is agonized by benoxacor. In some embodiments, the modified PYR/PYL protein comprises one or more sets of mutations corresponding to those set forth above. In some embodiments, such proteins are substantially identical to any of SEQ ID NOs:1-119 and/or comprise one or more consensus sequence selected from SEQ ID NOs:149-163. In some embodiments, the present invention provides for a polynucleotide encoding one or more of said modified PYR/PYL receptor polypeptides.

Additionally, in some embodiments of the present invention, a method of screening for modified PYR/PYL receptor polypeptides includes screening the modified polypeptide to determine whether it is agonized by benoxacor.

### BTH

In some cases, mutating one amino acid in the PYR/PYL receptor polypeptide results in activation of the modified receptor by BTH (Table 5). In some cases, mutating two or more amino acids in the PYR/PYL receptor polypeptide results in activation of the modified receptor by BTH. A non-limiting list of exemplary mutations and combinations of mutations that result in modified PYR/PYL receptor being agonized by BTH includes:
Q24R, K59R, I82T, F159L, and D161G
H115Y and F159S
F159L

Thus, in some embodiments a modified PYR/PYL receptor is agonized by BTH. In some embodiments, the modified PYR/PYL protein comprises one or more sets of mutations corresponding to those set forth above. In some embodiments, such proteins are substantially identical to any of SEQ ID NOs:1-119 and/or comprise one or more consensus sequence selected from SEQ ID NOs:149-163. In some embodiments, the present invention provides for a polynucleotide encoding one or more of said modified PYR/PYL receptor polypeptides.

Additionally, in some embodiments of the present invention, a method of screening for modified PYR/PYL receptor polypeptides includes screening the modified polypeptide to determine whether it is agonized by BTH.

Chemical agonists can be prepared by a variety of methods known to one of skill in the art, for example, those described in Comprehensive Organic Transformations, 2d ed., Richard C. Larock, 1999. The starting materials for the methods described above are commercially available (Sigma-Aldrich) or can be prepared by methods known to one of skill in the art.

In some embodiments, the agricultural chemical formulations contemplated are formulated for contacting to plants. The formulations can be suitable for treating plants or plant propagation material, such as seeds, in accordance with the present invention, e.g., in a carrier. Suitable additives include buffering agents, wetting agents, coating agents, polysaccharides, and abrading agents. Exemplary carriers include water, aqueous solutions, slurries, solids and dry powders (e.g., peat, wheat, bran, vermiculite, clay, pasteurized soil, many forms of calcium carbonate, dolomite, various grades of gypsum, bentonite and other clay minerals, rock phosphates and other phosphorous compounds, titanium dioxide, humus, talc, alginate and activated charcoal). Any agriculturally suitable carrier known to one skilled in the art would be acceptable and is contemplated for use in the present invention. Optionally, the formulations can also include at least one surfactant, herbicide, fungicide, pesticide, or fertilizer.

Contacting the agricultural chemical formulation to the mutated PYR/PYL receptor polypeptide can be performed in vitro (e.g., wherein the mutated PYR/PYL receptor polypeptide exists in a purified form or is expressed in yeast cells) or in vivo (e.g., wherein the mutated PYR/PYL receptor polypeptide is expressed by a plant). Contacting the agricultural chemical formulation to the mutated PYR/PYL receptor polypeptide in vitro can be performed using a variety of known methods, e.g., by applying the formulation to protein binding assays, mammalian or yeast two-hybrid assays, competition assays, or cell-based assays using other organisms.

Contacting the agricultural chemical formulation to the mutated PYR/PYL receptor polypeptide in vivo (e.g., to a plant) can be performed using a variety of known methods, e.g., by spraying, atomizing, dusting or scattering the compositions over the propagation material or brushing or pouring or otherwise contacting the compositions over the plant or, in the event of seed, by coating, encapsulating, or otherwise treating the seed. In an alternative to directly treating a plant or seed before planting, the formulations of the invention can also be introduced into the soil or other media into which the seed is to be planted. In some embodiments, a carrier is also used in this embodiment. The carrier can be solid or liquid, as noted above. In some embodiments peat is suspended in water as a carrier of the chemical agonist, and this mixture is sprayed into the soil or planting media and/or over the seed as it is planted.

### IV. Methods of Making Mutated PYR/PYL Receptor Polypeptides

Embodiments of the present invention provide for methods of making mutated PYR/PYL receptor polypeptides that are agonized by a chemical agonist that does not agonize a wild-type PYR/PYL receptor polypeptide. In some embodiments the method comprises mutagenizing the wild-type PYR/PYL receptor polypeptide, contacting one or more mutated PYR/PYL receptor polypeptides with the putative chemical agonist, and determining whether the chemical activates the one or more mutated PYR/PYL receptor polypeptides, wherein activation identifies the one or more mutated PYR/PYL receptor polypeptides as being agonized by the chemical.

Mutated PYR/PYL receptor polypeptides can be constructed by mutating the DNA sequences that encode the corresponding wild-type PYR/PYL receptor polypeptide (e.g., a wild-type PYR/PYL polypeptide of any of SEQ ID NOs:1-119 or a corresponding variant from which the mutant PYR/PYL receptor polypeptide of the invention is derived), such as by using techniques commonly referred to as site-directed mutagenesis. Nucleic acid molecules encoding the wild-type PYR/PYL receptor polypeptide can be mutated by a variety of polymerase chain reaction (PCR) techniques well-known to one of ordinary skill in the art. (See, e.g., PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, NY, 1990).

By way of non-limiting example, mutagenesis may be accomplished by means of error-prone PCR amplification (ePCR), which modifies PCR reaction conditions (e.g., using error-prone polymerases, varying magnesium or manganese concentration, or providing unbalanced dNTP ratios) in order to promote increased rates of error in DNA replication. Kits for ePCR mutagenesis are commercially available, such as the GeneMorph® PCR Mutagenesis kit (Stratagene) and Diversify® PCR Random Mutagenesis Kit (Clontech). Briefly, DNA polymerase (e.g., Taq polymerase), salt (e.g., MgCl2, MgSO4, or MnSO4), dNTPs in unbalanced ratios, reaction buffer, and DNA template are combined and subjected to standard PCR amplification according to manufacturer's instructions. Following ePCR amplification, the reaction products are cloned into a suitable vector to construct a mutagenized library, which can then be transformed into suitable cells (e.g., yeast cells) for subsequent screening (e.g., via a two-hybrid screen) as described below.

Alternatively, mutagenesis can be accomplished by recombination (i.e. DNA shuffling). Briefly, a shuffled mutant library is generated through DNA shuffling using in vitro homologous recombination by random fragmentation of a parent DNA followed by reassembly using PCR, resulting in randomly introduced point mutations. Methods of performing DNA shuffling are known in the art (see, e.g., Stebel, S. C. et al., Methods Mol Biol 352:167-190 (2007)).

Optionally, multiple rounds of mutagenesis may be performed in order to improve the efficiency of mutant proteins isolated. Thus, in some embodiments, PYR/PYL mutants isolated from ePCR and subsequent screening may be pooled and used as templates for later rounds of mutagenesis.

### V. Screening for Agonism of Mutated PYR/PYL Receptor Polypeptides

Embodiments of the present invention also provide for methods of screening putative chemical agonists to determine whether the putative agonist agonizes a mutated PYR/PYL receptor polypeptide, but does not significantly agonize a wild-type PYR/PYL receptor polypeptide, when the putative agonist is contacted to the PYR/PYL receptor polypeptide. As used herein, an agent “agonizes” a PYR/PYL receptor protein if the presence of the agent results in activation or up-regulation of activity of the receptor, e.g., to increase downstream signaling from the PYR/PYL receptor. For the present invention, an agent agonizes a PYR/PYL receptor if, when the agent is present at a concentration no greater than 200 μM, contacting the agent to the PYR/PYL receptor results in activation or up-regulation of the activity of the PYR/PYL receptor. If an agent does not induce activation or up-regulation of a PYR/PYL receptor protein's activity when the agent is present at a concentration no greater than 200 μM, then the agent does not significantly agonize the PYR/PYL receptor. As used herein, “activation” requires a minimum threshold of activity to be induced by the agent. Determining whether this minimum threshold of activity has been met can be accomplished, e.g., by using an enzymatic phosphatase assay that sets a minimum value for the level of enzymatic activity that must be induced, or by using an enzymatic phosphatase assay in the presence of a colorimetric detection reagent (e.g., para-nitrophenylphosphate) wherein the minimum threshold of activity has been met if a color change is observed.

A number of different screening protocols can be utilized to identify chemical agents that agonize a mutated PYR/PYL receptor polypeptide but not a wild-type PYR/PYL receptor polypeptide. Screening can take place using isolated, purified or partially purified reagents. In some embodiments, purified or partially purified PYR/PYL polypeptide can be used.

Alternatively, cell-based or plant-based methods of screening can be used. For example, cells that naturally express a wild-type PYR/PYL receptor polypeptide or that recombinantly express a wild-type or mutated PYR/PYL receptor polypeptide can be used. In some embodiments, the cells used are plant cells, animal cells, bacterial cells, fungal cells, including but not limited to yeast cells, insect cells, or mammalian cells. In general terms, the screening methods involve screening one or more chemical agents to identify an agent that agonizes the activity of a mutated PYR/PYL receptor polypeptide (e.g., activating the mutated PYR/PYL receptor polypeptide or increasing expression of the mutated PYR/PYL receptor polypeptide or of a transcript encoding a mutated PYR/PYL receptor polypeptide), but that does not agonize the activity of a wild-type PYR/PYL receptor polypeptide. Optionally, the screening method may involve two screening processes: first, screening a plurality of putative agonists to identify compounds that weakly interact with a wild-type PYR/PYL receptor polypeptide (“weak ligands”), then screening those weak ligands against wild-type PYR/PYL receptor polypeptide and a plurality of mutagenized PYR/PYL receptor polypeptides to determine which mutated PYR/PYL receptor polypeptides are agonized by weak ligands and which weak ligands selectively agonize only mutated PYR/PYL receptor polypeptides and not wild-type PYR/PYL receptor polypeptides.

### Binding Assays

Optionally, preliminary screens can be conducted by screening for agents capable of binding to a wild-type PYR/PRL receptor polypeptide. Pre-selection of weak-binding ligands improves the frequency of isolating mutated PYR/PYL receptor polypeptides that are agonized by the agent, presumably because fewer alterations of the ligand binding site are required to achieve molecular recognition.

Binding assays can involve contacting a wild-type PYR/PYL receptor polypeptide with one or more chemical agents and allowing sufficient time for the protein and chemical agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to the PYR/PYL polypeptide or displacement of labeled substrates (e.g., labeled agrochemical). The PYR/PYL polypeptide protein utilized in such assays can be naturally expressed, cloned or synthesized.

### Agonist Assays

Agonist assays can involve screening putative chemical agonists (which may or may not have been pre-selected as weak binding ligands) to determine which putative agonists agonize at least one mutated PYR/PYL receptor polypeptides but not a wild-type PYR/PYL receptor polypeptide, and/or screening mutagenized PYR/PYL receptor polypeptides with putative chemical agonists (which may or may not have been pre-selected as weak binding ligands) to determine which mutagenized PYR/PYL receptor polypeptides are agonized by the putative agonist.

Any number of assays can be used to screen for agonists of mutated PYR/PYL receptor polypeptides. One activity assay involves testing whether a putative agonist can induce binding of a mutated PYR/PYL protein to a type 2 protein phosphatase (PP2C) polypeptide in an agonist-specific fashion. Mammalian or yeast two-hybrid approaches (see, e.g., Bartel, P. L. et. al. Methods Enzymol, 254:241 (1995)) can be used to identify polypeptides or other molecules that interact or bind when expressed together in a cell. In some embodiments, agents that agonize a mutated PYR/PYL receptor polypeptide, but not a wild-type PYR/PYL receptor polypeptide, are identified in a two-hybrid assay between a PYR/PYL polypeptide and a type 2 protein phosphatase (PP2C) polypeptide, wherein an agonist is identified as an agent that activates or enables binding of the PYR/PYL polypeptide and the PP2C polypeptide. Thus, the two polypeptides bind in the presence, but not in the absence of the agent. Optionally, both positive and negative selection schemes can be utilized in the two-hybrid assay. For example, a yeast two-hybrid assay may utilize a URA3 reporter strain to conduct both positive and negative selection; growth of the URA strain in the absence of exogenously supplied uracil enables positive selection for mutants that improve agonist responsiveness (i.e. agonist-promoted protein-protein interaction), while growth on FOA (5-fluoro-orotic acid, which is metabolized by URA3 to a toxic metabolite) allows selection against mutants that promote agonist response (e.g. to remove mutants that lead to constitutive, i.e. unliganded, interactions).

Screening for a compound that increases the expression of a mutated PYR/PYL receptor polypeptide, but not a wild-type PYR/PYL receptor polypeptide, is also provided. Screening methods generally involve conducting cell-based or plant-based assays in which test compounds are contacted with one or more cells expressing PYR/PYL polypeptide, and then detecting an increase in PYR/PYL expression (either transcript or translation product). Assays can be performed with cells that naturally express wild-type PYR/PYL or in cells recombinantly altered to express mutated or wild-type PYR/PYL. Various controls can be conducted to ensure that an observed activity is authentic, including running parallel reactions with cells that lack the reporter construct or by not contacting a cell harboring the reporter construct with test compound.

Agents and mutated PYR/PYL receptor polypeptides that are initially identified by any of the foregoing screening methods can be further tested to validate the apparent activity and/or determine other biological effects of the agent and/or mutated PYR/PYL receptor polypeptide. In some cases, the identified agent and/or mutated PYR/PYL receptor polypeptide is tested for the ability to effect plant stress (e.g., drought tolerance), seed germination, or another phenotype affected by ABA. A number of such assays and phenotypes are known in the art and can be employed according to the methods of the invention.

### VI. Recombinant Expression Vectors

Once a polynucleotide sequence encoding a mutated PYR/PYL receptor polypeptide is obtained, it can also be used to prepare an expression cassette for expressing the mutated PYR/PYL receptor polypeptide in a transgenic plant, directed by a heterologous promoter. Increased expression of mutated PYR/PYL polynucleotide is useful, for example, to produce plants that will be able to respond to a chemical agonist that does not agonize endogenous PYR/PYL receptor protein, thereby enhancing abiotic stress resistance.

Any of a number of means well known in the art can be used to drive mutated PYR/PYL activity or expression in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, the mutated PYR/PYL polynucleotide can be expressed constitutively (e.g., using the CaMV 35S promoter).

To use a polynucleotide sequence for a mutated PYR/PYL receptor polypeptide in the above techniques, recombinant DNA vectors suitable for transformation of plant cells are prepared. Techniques for transforming a wide variety of higher plant species are well known and described in the technical and scientific literature. See, e.g., Weising et al. Ann. Rev. Genet. 22:421-477 (1988). A DNA sequence coding for the mutated PYR/PYL receptor polypeptide preferably will be combined with transcriptional and translational initiation regulatory sequences which will direct the transcription of the sequence from the gene in the intended tissues of the transformed plant.

For example, a plant promoter fragment may be employed to direct expression of the mutated PYR/PYL polynucleotide in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the mutated PYR/PYL receptor protein in a specific tissue (tissue-specific promoters) or may be otherwise under more precise environmental control (inducible promoters). Examples of tissue-specific promoters under developmental control include promoters that initiate transcription only in certain tissues, such as leaves or guard cells (including but not limited to those described in WO/2005/085449; U.S. Pat. No. 6,653,535; Li et al., Sci China C Life Sci. 2005 April; 48(2):181-6; Husebye, et al., Plant Physiol, April 2002, Vol. 128, pp. 1180-1188; and Plesch, et al., Gene, Volume 249, Number 1, 16 May 2000, pp. 83-89(7)). Examples of environmental conditions that may affect transcription by inducible promoters include anaerobic conditions, elevated temperature, or the presence of light.

If proper protein expression is desired, a polyadenylation region at the 3′-end of the coding region should be included. The polyadenylation region can be derived from a naturally occurring PYR/PYL gene, from a variety of other plant genes, or from T-DNA.

The vector comprising the sequences (e.g., promoters or PYR/PYL coding regions) will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, the mutated PYR/PYL nucleic acid sequence is expressed recombinantly in plant cells. A variety of different expression constructs, such as expression cassettes and vectors suitable for transformation of plant cells can be prepared. Techniques for transforming a wide variety of higher plant species are well known and described in the technical and scientific literature. See, e.g., Weising et al. Ann. Rev. Genet. 22:421-477 (1988). A DNA sequence coding for a PYR/PYL protein can be combined with cis-acting (promoter) and trans-acting (enhancer) transcriptional regulatory sequences to direct the timing, tissue type and levels of transcription in the intended tissues of the transformed plant. Translational control elements can also be used.

Embodiments of the present invention also provide for a mutated PYR/PYL nucleic acid operably linked to a promoter which, in some embodiments, is capable of driving the transcription of the PYR/PYL coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal.

### Constitutive Promoters

A promoter fragment can be employed to direct expression of a mutated PYR/PYL nucleic acid in all transformed cells or tissues, e.g., as those of a regenerated plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding a mutated PYR/PYL receptor protein (Comai et al., Plant Mol. Biol. 15:373 (1990)).

Other examples of constitutive promoters include the 1′- or 2′-promoter derived from T-DNA of Agrobacterium tumafaciens (see, e.g., Mengiste (1997) supra; O'Grady (1995) Plant Mol. Biol. 29:99-108); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf Plant Mol. Biol. 29:637-646 (1995).

### Inducible Promoters

Alternatively, a plant promoter may direct expression of the mutated PYR/PYL polynucleotide under the influence of changing environmental conditions or developmental conditions. Examples of environmental conditions that may effect transcription by inducible promoters include anaerobic conditions, elevated temperature, drought, or the presence of light.

Such promoters are referred to herein as “inducible” promoters. For example, the invention can incorporate a drought-specific promoter such as a drought-inducible promoter of maize (e.g., the maize rab17 drought-inducible promoter (Vilardell et al. (1991) Plant Mol. Biol. 17:985-993; Vilardell et al. (1994) Plant Mol. Biol. 24:561-569)); or alternatively a cold, drought, and high salt inducible promoter from potato (Kirch (1997) Plant Mol. Biol. 33:897-909).

Alternatively, plant promoters which are inducible upon exposure to plant hormones, such as auxins, are used to express the mutated PYR/PYL polynucleotide. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

Plant promoters inducible upon exposure to chemicals reagents that may be applied to the plant, such as herbicides or antibiotics, are also useful for expressing the mutated PYR/PYL polynucleotide. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. A PYR/PYL coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Examples of useful inducible regulatory elements include copper-inducible regulatory elements (Mett et al., Proc. Natl. Acad. Sci. U.S.A. 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. U.S.A. 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

### Tissue-Specific Promoters

Alternatively, the plant promoter may direct expression of the mutated PYR/PYL polynucleotide in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue, or epidermis or mesophyll. Reproductive tissue-specific promoters may be, e.g., ovule-specific, embryo-specific, endosperm-specific, integument-specific, seed and seed coat-specific, pollen-specific, petal-specific, sepal-specific, or some combination thereof. In some embodiments, the promoter is cell-type specific, e.g., guard cell-specific.

Other tissue-specific promoters include seed promoters. Suitable seed-specific promoters are derived from the following genes: MAC1 from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmyc1 from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express polynucleotides encoding mutated PYR/PYL receptor polypeptides. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes that exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are kn1-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, the mutated PYR/PYL polynucleotide is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai (1995) Proc. Natl. Acad. Sci. U.S.A. 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

### VII. Production of Transgenic Plants

As detailed herein, embodiments of the present invention provide for transgenic plants comprising recombinant expression cassettes for expressing a mutant PYR/PYL receptor protein as described herein in a plant. In some embodiments, a transgenic plant is generated that contains a complete or partial sequence of a polynucleotide that is derived from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a PYR/PYL coding sequence driven by a heterologous promoter may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of mutated PYR/PYL is encompassed by the invention, generally expression of construction of the invention will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. U.S.A. 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. U.S.A. 80:4803 (1983).

Transformed plant cells derived by any of the above transformation techniques can be cultured to regenerate a whole plant that possesses the transformed genotype and thus the desired phenotype such as enhanced abiotic stress resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The expression cassettes of the invention can be used to confer abiotic stress resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malta, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. In some embodiments, the plant is selected from the group consisting of rice, maize, wheat, soybeans, cotton, canola, turfgrass, and alfalfa. In some embodiments, the plant is an ornamental plant. In some embodiment, the plant is a vegetable- or fruit-producing plant.

Those of skill will recognize that a number of plant species can be used as models to predict the phenotypic effects of transgene expression in other plants. For example, it is well recognized that both tobacco (Nicotiana) and Arabidopsis plants are useful models of transgene expression, particularly in other dicots.

In some embodiments, the plants of the invention have enhanced sensitivity to certain chemical agonists compared to plants are otherwise identical except for expression of the mutated PYR/PYL receptor polypeptide. Sensitivity to agonists that agonize the PYR/PYL family of ABA receptors can be monitored by observing or measuring any phenotype mediated by ABA. Those of skill in the art will recognize that ABA is a well-studied plant hormone and that ABA mediates many changes in characteristics, any of which can be monitored to determine whether ABA sensitivity has been modulated. In some embodiments, modulated ABA sensitivity is manifested by altered timing of seed germination or altered stress (e.g., drought) tolerance.

Abiotic stress resistance can assayed according to any of a number of well-known techniques. For example, for drought tolerance, plants can be grown under conditions in which less than optimum water is provided to the plant. Drought resistance can be determined by any of a number of standard measures including turgor pressure, growth, yield, and the like.

### EXAMPLES

The following examples are offered to illustrate, but not to limit the claimed invention.

### Example 1

### Isolation of PYR1 Orthogonal Receptors

For isolating mutated (orthogonal) PYR/PYL receptors, a suitable target ligand is first identified. Next, receptor mutagenesis and selection experiments are used to identify orthogonal receptors that respond to the target orthogonal ligand. In general, the higher the starting affinity of the target ligand for the receptor prior to mutagenesis, the fewer the number of mutations that will be needed to realize target recognition. Proteins with promiscuous ligand-binding pockets are inherently better starting points for engineering efforts than those with pockets that are highly selective, since they are likely to make weak contacts with a greater number of ligands than a highly selective binding pocket. Furthermore, a receptor protein whose function can be measured in a heterologous host such as S. cerevisiae is a preferred target for receptor engineering, because such assays allow large numbers of variant receptors to be screened rapidly.

### Methods and Results

To isolate PYR1 orthogonal receptors, a two-step process was used. First, a large collection of compounds was screened to identify compounds that weakly interact with receptors, defined as such by their ability to displace receptor-bound ABA in competition experiments. Once weak binders were identified, those with the most desirable properties from an end-use perspective were used as targets for iterative mutagenesis and selection schemes. Pre-selection of weak-binding ligands improves the frequency of isolating new receptor proteins, because fewer alterations of the ligand binding site are required to achieve molecular recognition. However, pre-selection is not a prerequisite for success.

Pre-selection of ligands. 74 structurally diverse agrochemicals were obtained, selected from a larger set of approximately 1500 commercially available agrochemicals. These 74 compounds were tested for their ability to reduce ABA-promoted PYR/PYL-PP2C protein-protein interactions in suitable yeast strains when added at 1000-fold excess relative to ABA (i.e. 100 nM ABA and 100 μM test chemical). This pilot screen revealed that the closely related herbicides bromoxynil and its chloro analog chloroxynil, the rodenticide coumatetralyl, the herbicide dicholbenil, and the fungicide fenhexamid all compete for ABA. This observed hit rate of 5 out of 74 screened compounds demonstrates that weak binding to the PYR/PYL ligand binding pocket is common, consistent with observations that START proteins have promiscuous ligand binding pockets (Mogensen, J. E. et al., Journal of Biological Chemistry 277:23684-23692 (2002)).

PCR-based Mutagenesis of PYR1. To identify PYR/PYL mutations that alter agonist responsiveness, the coding sequence for PYR1 was mutated by error-prone PCR using established protocols (Lin-Goerke et al., Biotechniques 23:409-412 (1997)) and cloned into the yeast two hybrid vector pBD-GAL4, yielding a library of ˜70,000 mutants (named ePCR1 library). This plasmid library was amplified in E. coli and then transformed into S. cerevisiae strain MAV99 co-transformed with a Ga14 activation domain (AD)-PP2C fusion protein encoded by the plasmid pAD-HAB1, as described previously (Park et al., Science 324:1068-1071 (2009)). MAV99 (Vidal et al., Proc Natl Acad Sci U.S.A. 93:10315-10320 (1996)) is a reverse two hybrid strain that contains a Ga14 activated URA3 reporter gene. This strain will not grow in the absence of exogenously supplied uracil. However, if the strain expresses a PYR1 mutant that enables a protein-protein interaction between PYR1 and HAB 1, the URA3 reporter gene is activated, which enables strain growth and subsequent colony formation. Thus, co-expression of BD-PYR1 and AD-HAB1 in the MAV99 strain enables a positive selection scheme for PYR1 mutations that allow non-natural agonists to promote the PYR1-HAB1 interaction, which can be observed as agonist promoted growth in the absence of added uracil. In addition, inclusion of the compound 5-fluoro-orotic acid (FOA, which is metabolized by URA3 to a toxic metabolite) in growth media, instead of uracil, allows selection against mutations that confer BD-PYR1-AD-HAB 1 interactions in an agonist-independent fashion (“constitutive mutations”). Constitutive mutations are not desirable in this screening strategy because a target agrochemical molecule would not regulate the activity of a constitutive mutant. Because both regulated and constitutive PYR1 mutants will enable growth of the HAB 1-AD expressing MAV99 strain in the absence of exogenously supplied uracil, it is beneficial to utilize both positive and negative selection schemes.

Isolation of agrochemical responsive mutants. To identify PYR1 mutations that confer responsiveness to non-natural agrochemical agonists, the ePCR1 library was transformed into the MAV99/pAD-HAB 1 strain to create a library called “A”. The A library was then grown on media containing 0.15% FOA. This created a mutant library called “A′”, which is a library of PYR1 mutants with reduced numbers of constitutive mutations.

The A′ library was then used for a variety of selection experiments using different agrochemicals. For example, for the isolation of fenhexamid responsive mutations 300,000 A′ cells were plated onto growth media lacking uracil and containing 200 μM fenhexamid. After several days incubation, ˜80 colonies appeared and were individually collected and retested on growth media either containing or lacking fenhexamid. Colonies that grew on plates lacking fenhexamid contain constitutive mutations that were not eliminated during the preparation of the A′ library and were discarded. The PYR1 coding sequences of the remaining ˜30 mutations were sequenced, which revealed that 7 distinct mutant sequences (Table 1) had been isolated:

### 27-18: S47P, K59R, Y120H

### 27-24: K59R, Y120H, V144A

### 27-31: P42S, K59R, Y120C

### 27-28: P42S, K59R, Y120C, T124M

### 27-9: P42S, K59R, Y120C, E154G

### 27-36: E12G, E43G, K59R, 1110S, N133D

### 27-14: E12G, L25R, E43G, K59R, 1110S, N133D

TABLE 1
Fenhexamid-responsive PYR/PYL receptor polypeptide mutants

| | | | Growth* on | | |
|---|---|---|---|---|---|
| | | | [Fenhexamid] | | |
| Clone # | Mutations Present | Source | 0 μM | 200 μM | SEQ ID NO |
| 27-18 | S47P, K59R, Y120H | ePCR1 | − | +++ | 124 |
| 27-24 | K59R, Y120H, V144A | ePCR1 | − | +++ | 125 |
| 27-31 | P42S, K59R, Y120C | ePCR1 | − | +++ | 126 |
| 27-28 | P42S, K59R, Y120C, T124M | ePCR1 | − | +++ | 127 |
| 27-9 | P42S, K59R, Y120C, E154G | ePCR1 | − | +++ | 128 |
| 27-36 | E12G, E43G, K59R, I110S, N133D | ePCR1 | − | +++ | 129 |
| 27-14 | E12G, L25R, E43G, K59R, I110S, | ePCR1 | − | +++ | 130 |
| | N133D | | | | |

ePCR1 = error prone PCR mutagenesis library of PYR1, 70,000 clones starting size.
*Growth was measured on media lacking uracil and using the reporter strain MAV99, which only grows when an agonist-promoted protein-protein interaction between PYR1-GAL4 BD and HAB1-GAL4 AD reconstitutes GAL4 activity and enables expression of the strain's URA3 gene.

Fenhexamid responsiveness of the PYR1 mutants was confirmed by yeast two-hybid for representative mutants. Plasmids for three representative mutant clones (27-9, 27-18, and 27-36) were isolated from the primary yeast cells and transformed into a pAD-HAB1 expressing YRG-2 reporter strain (Park et al., Science 324:1068-1071 (2009)) in which Ga14 drives expression of a LacZ reporter gene to allow colorimetric indication of agonist response. Fenhexamid was applied at concentrations of 1, 5, 10, 25, and 50 μM. Mutant 27-18 strongly responded to fenhexamid at all the concentrations tested, as determined by the strong blue (LacZ-positive) staining. Mutant 27-9 strongly responded to fenhexamid at 5, 10, 25, and 50 μM concentrations, and was weakly positive at 1 μM. Mutant 27-36 was responsive to fenhexamid at 25 and 50 μM concentrations.

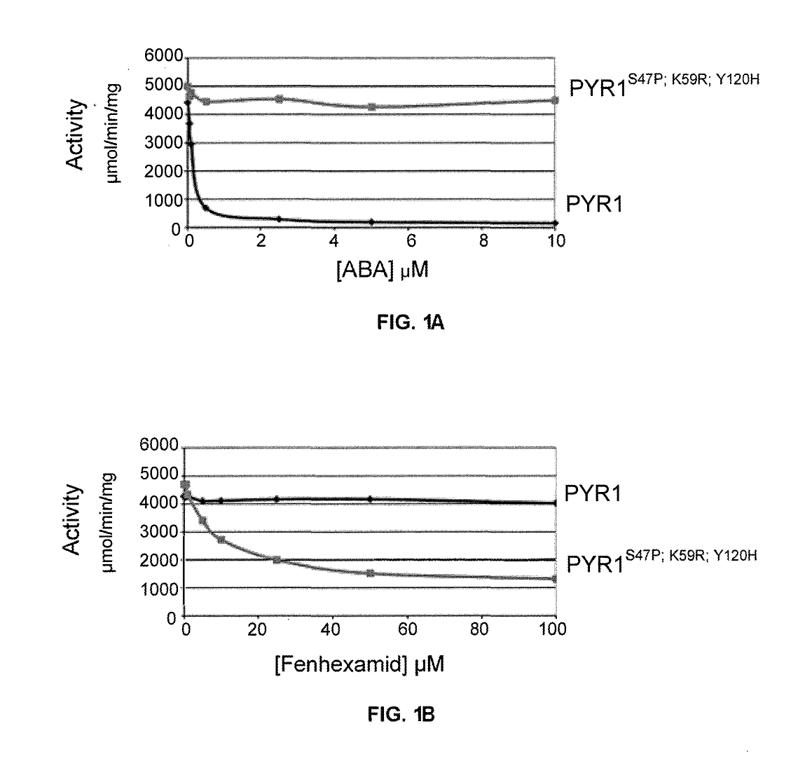

To probe orthogonal receptor-ligand interactions further, recombinant protein was expressed for the fenhexamid-responsive mutant 27-18, which has three mutations in PYR1. 6×-His-PYR1(27-18) protein was expressed and purified alongside wild-type PYR1 protein. Both proteins were tested for their ability to inhibit PP2C activity in response to increasing concentrations of either ABA or fenhexamid using phosphatase activity assays, in which activation of the PYR/PYL receptor is monitored by inhibition of PP2C activity (FIG. 1). Phosphatase activity assays were performed as described in Park et al., Science 324:1068-1071 (2009), with the minor modification that the assay buffer utilized 10 mM Mn++ instead of Mg++; it was found that this modification enhanced HAB1's specific activity ˜10 fold. It was found that ABA failed to activate mutant 27-18, but efficiently activated wild-type PYR1 protein (FIG. 1A), therefore demonstrating that mutant 27-18 is insensitive to ABA. Furthermore, mutant 27-18 was activated by fenhexamid whereas wild-type PYR1 protein was not (FIG. 1B), therefore demonstrating that mutant PYR/PYL receptor polypeptides can be used to control PP2C activity in response to fenhexamid.

Screens for bromoxynil (Table 2), dichlobenil (Table 3), and benoxacor (Table 4) responsive PYR1 mutants were conducted as described above for fenhexamid, each utilizing 200 μM test compound and the A′ library, generated as described above. Yeast two-hybrid assay confirmed bromoxynil responsiveness for representative mutant clones 74A-1 and 74A-2. Bromoxynil was applied at concentrations of 5, 10, 25, and 50 μM. Both mutants 74A-1 and 74A-2 were responsive to bromoxynil at concentrations of 10, 25, and 50 μM.

TABLE 2
Bromoxynil-responsive PYR/PYL receptor polypeptide mutants

| | | | Growth* on | | | |
|---|---|---|---|---|---|---|
| | | | [Bromoxynil] | | | SEQ |
| Clone | | | 0 | 0.5 | 200 | ID |
| # | Mutations Present | Source | μM | μM | μM | NO |
| 74A-12 | T57A, K59R | ePCR1 | − | ND | ++ | 131 |
| 74A-24 | R50G, K59R, E141Q | ePCR1 | − | ND | ++ | 132 |
| 74A-1 | K59R, H60R, N151D | ePCR1 | − | ND | ++ | 133 |
| 74A-4 | K59R, H60R, E102G, | ePCR1 | − | ND | ++ | 134 |
| | T125A, E141D | | | | | |
| 74A-13 | K59R, I82N | ePCR1 | − | ND | ++ | 135 |
| 74A-2 | K59R, S92T | ePCR1 | − | ND | ++ | 136 |
| 74A-15 | K59R, S92T | ePCR1 | − | ND | ++ | 137 |
| 74B-1 | H21Y, K59R, H60R, | Shuffled | − | ++ | ND | 138 |
| | S92T, R116K | First Round | | | | |
| 74B-7 | P41L, K59R, H60R | Shuffled | − | ++ | ND | 139 |
| | | First Round | | | | |

ePCR1 = error prone PCR mutagenesis library of PYR1, 70,000 clones starting size; Shuffled First Round = PYR1 shuffled mutant library made using isolated ePCR mutants.
*Growth was measured on media lacking uracil and using the reporter strain MAV99, which only grows when an agonist-promoted protein-protein interaction between PYR1-GAL4 BD and HAB1-GAL4 AD reconstitutes GAL4 activity and enables expression of the strain's URA3 gene.

TABLE 3
Dichlobenil-responsive PYR/PYL receptor polypeptide mutants

| | | | Growth* on | | |
|---|---|---|---|---|---|
| | | | [Dichlobenil] | | |
| Clone # | Mutations Present | Source | 0 μM | 200 μM | SEQ ID NO |
| 68-1 | E94D | ePCR1 | − | ++ | 140 |
| 68-2 | R37Q, F71S | ePCR1 | − | ++ | 141 |
| 68-8 | D26G | ePCR1 | − | ++ | 142 |
| 68-3 | P27L, K59R, K63N | ePCR1 | − | ++ | 143 |
| 68-17 | K59R | ePCR1 | − | ++ | 144 |

ePCR1 = error prone PCR mutagenesis library of PYR1, 70,000 clones starting size.
*Growth was measured on media lacking uracil and using the reporter strain MAV99, which only grows when an agonist-promoted protein-protein interaction between PYR1-GAL4 BD and HAB1-GAL4 AD reconstitutes GAL4 activity and enables expression of the strain's URA3 gene.

TABLE 4
Benoxacor-responsive PYR/PYL receptor polypeptide mutants

| | | | Growth* on | | |
|---|---|---|---|---|---|
| Clone | | | [Benoxacor] | | |
| # | Mutations Present | Source | 0 μM | 200 μM | SEQ ID NO |
| 129-2 | I110T, E114D, V138M | ePCR1 | − | + | 145 |
| 127-1 | K59R, N119Y | ePCR1 | − | + | 146 |

ePCR1 = error prone PCR mutagenesis library of PYR1, 70,000 clones starting size.
*Growth was measured on media lacking uracil and using the reporter strain MAV99, which only grows when an agonist-promoted protein-protein interaction between PYR1-GAL4 BD and HAB1-GAL4 AD reconstitutes GAL4 activity and enables expression of the strain's URA3 gene.

Improvement of primary mutants using DNA shuffling. Recombining existing mutations or variants using DNA shuffling is a proven method to rapidly improve protein function and activity (Stemmer, Nature 370:389-391 (1994)). To improve the isolated bromoxynil responsive PYR1 mutants, plasmid DNA for the 7 mutant sequences identified by screening the A′ library (74A-12, 74A-24, 74A-1, 74A-4, 74A-13, 74A-2 and 74A-15) were combined with an equimolar amount of plasmid for the original PYR1 ePCR1 mutant library. The addition of ePCR1 DNA to the shuffling reaction enabled new mutations to be introduced into existing mutations and therefore increases potential sequence diversity. The mixed DNAs were shuffled using an established protocol (Muller et al., Nucleic Acids Res 33:e117 (2005)) and the shuffled PCR product DNA was cloned into pBD-GAL4 to generate a ˜50,000 clones which were collected and named the “B” library, which amplified in E. coli and was introduced into yeast strain MAV99 co-transformed with pAD-HAB 1. These cells were then grown on 0.15% FOA to remove constitutive mutants, yielding a “B” library. The B′ library was then grown on plates lacking added uracil and containing 0.5 μM bromoxynil. Two unique non-constitutive clones were isolated (74B-1 and 74B-7) (Table 2).

Given the plasticity of PYR1 indicated by these experiments, we sought to establish if an orthogonal receptor could be isolated for a target ligand that was not prescreened for weak binding using the yeast competition assays described above. The same screening methodology as described above was used to screen for mutants that respond to BTH (acibenzolar-s-methyl). This screen resulted in the successful isolation of three different PYR1 mutants that respond to BTH (Table 5). Yeast two-hybrid assay confirmed BTH responsiveness for representative mutant clone BTH-9. Bromoxynil was applied at concentrations of 25, 50, 100, and 200 μM, and mutant BTH-9 was responsive to BTH at concentrations of 50, 100, and 200 μM. Thus, the plasticity of PYR1 enables many orthogonal receptor variants to be isolated.

TABLE 5
BTH-responsive PYR/PYL receptor polypeptide mutants

| | | | Growth* | | |
|---|---|---|---|---|---|
| | | | on [BTH] | | |
| Clone # | Mutations Present | Source | 0 μM | 200 μM | SEQ ID NO |
| BTH-1 | H115Y, F159S | ePCR1 | − | + | 147 |
| BTH-9 | F159L | ePCR1 | − | + | 148 |
| BTH- | Q24R, K59R, I82T, | ePCR1 | − | + | 164 |
| An7 | F159L, D161G | | | | |

ePCR1 = error prone PCR mutagenesis library of PYR1, 70,000 clones starting size.
*Growth was measured on media lacking uracil and using the reporter strain MAV99, which only grows when an agonist-promoted protein-protein interaction between PYR1-GAL4 BD and HAB1-GAL4 AD reconstitutes GAL4 activity and enables expression of the strain's URA3 gene.

### Example 2

### Mutations at the K59 Position Sensitize PYR to Diverse Orthogonal Ligands

Inspection of the screening data in Tables 1-5 revealed that receptors containing a mutation at the K59 position were isolated at least once for all chemicals screened. In most cases, a K59R mutation was present in the majority of orthogonal receptors isolated for each chemical screened. This surprising observation suggests that K59 is a control point that can be targeted beneficially to engineer effective orthogonal receptors. Two plausible hypotheses for the frequent occurrence of a mutation at the position corresponding to amino acid K59 of PYR1 are the “brake” hypothesis and the “pocket shape” hypothesis. The brake hypothesis proposes that the K59 residue functions as part of a “braking” mechanism to help keep receptors in their “off” state in the absence of bound ABA; therefore, mutations at K59 may disrupt a control mechanism that keeps receptor activation linked to ABA binding and prevents receptors from being activated by non-natural ligands. The pocket shape hypothesis proposes that mutations at the position corresponding to K59 alters the PYR/PYL receptor's binding pocket to create a new pocket surface that facilitates interactions between PYR/PYL and orthogonal ligands to improve binding affinity.

To test these two hypotheses, a series of K59 mutants were constructed in PYR1 and examined for their sensitivity to the native ligand ABA and the orthogonal ligand dichlobenil (Table 6). Dichlobenil was chosen as a model orthogonal ligand for these studies because it was observed that it is a weak PYR/PYL agonist (i.e., it can weakly activate receptors at concentrations of 200 μM or higher). Dichlobenil therefore provides a useful test molecule with which to probe a mutant's sensitivity to an orthogonal ligand.

TABLE 6
Responsiveness of K59 mutations to the orthogonal ligand dichlobenil

| Mutation | ABA [10 μM] | Dichlobenil [50 μM] |
|---|---|---|
| WT | + | − |
| K59A | − | + |
| K59C | − | + |
| K59F | + | + |
| K59G | − | + |
| K59H | − | + |
| K59I | − | − |
| K59L | − | + |
| K59M | − | + |
| K59P | − | − |
| K59R | − | + |
| K59S | − | + |
| K59T | − | + |
| K59V | − | + |
| K59Y | − | + |
| K59N | + | + |
| K59W | − | + |

As shown in Table 6, wild-type PYR1 was not activated by 50 μM dichlobenil. However, 14 of the 16 K59 substitution mutants constructed were activated by 50 μM dichlobenil. Thus, the majority of mutations at K59 enhance dichlobenil sensitivity. These results, coupled with the prevalence of K59R mutations isolated in the screens described herein (e.g., in Example 1 and in Tables 1-5), suggest that many mutations in K59 are beneficial for constructing receptors that are activated by non-native (i.e., orthogonal) ligands, and further suggest that K59 is more likely to be acting according to the brake hypothesis than the pocket shape hypothesis, as the pocket shape hypothesis predicts that an altered pocket surface that facilitates binding of one orthogonal ligand is unlikely to also be the shape that facilitates another orthogonal ligand's binding.

### Discussion

The receptor mutations isolated in the collection of mutants are preferentially located in residues whose side chains point into the ligand-binding pocket of PYR1. This is not surprising, as the surface of the ligand-binding pocket of PYR1 must be resculpted to make contact with new ligands. Additionally, mutations isolated in orthogonal receptors (i.e. K59R, S92T) mutate invariant positions within the PYR/PYL ABA receptor family. These positions are invariant because they are involved in ABA recognition and under strong natural selection for proper ABA-binding. Since mutations in conserved ABA-binding residues are known to reduce ABA responsiveness, orthogonal receptors can be expected to be insensitive to ABA when expressed in plant cells. An advantage of this feature is that over-expression of orthogonal receptors should not lead to activation of ABA signaling in the absence of the controlling orthogonal ligand.

The biochemical function of PYR1, and PYR/PYL proteins in general, is to inhibit PP2C activity. This can be measured in live cells using the yeast two hybrid or other cell-based methods. It can also be measured in vitro using enzymatic phosphatase assays in the presence of a colorimetric detection reagent (for example, para-nitrophenylphosphate). We note that the yeast-based assay used above provides an indirect indicator of ligand binding. It is possible that some compounds screened may reduce ABA responsiveness of yeast strains without directly binding in its central pocket. To address this potential limitation, one can use in vitro competition assays, or cell based assays using other organisms, as alternate approaches for identifying weak binding target compounds.

### Example 3

### Improvement of Fenhexamid Receptor Sensitivity

### Generating Fenexamid-Responsive PYR1 Variants

As detailed in Example 1, the screening of the ePCR1 mutant library for fenhexamid responsive mutants led to the isolation of several mutant PYR1 receptors that respond to fenhexamid (Table 1). To improve fenhexamid receptor sensitivity, DNA shuffling was employed using the same general experimental scheme outlined previously. Briefly, equimolar amounts of plasmid DNA for the fenhexamid receptors shown in Table 1 were pooled and combined with an equimolar amount of the ePCR1 library DNA. The pooled templates were utilized for DNA shuffling, which was conducted as described in Example 1. A library (named “27”) of ˜400,000 shuffled variants was prepared. The DNA for this library was introduced into the MAV99 pAD-HAB 1 yeast strain as described above and the resulting yeast cells collected and grown on FOA-containing plates to reduce constitutive mutants in the library, yielding the 27B′ library. The 27B′ library was plated onto media lacking uracil but containing 20 μM fenhexamid. ˜50 positives were selected from these plates and subsequently retested on media lacking uracil to distinguish constitutive mutants (i.e., false positives) from those mutants that grow specifically in response to fenhexamid (i.e., true positives). The PYR1 coding sequences for the true positives were sequenced to yield the following series of fenhexamid-responsive PYR1 variants (Table 7):

TABLE 7
Fenhexamid-responsive PYR/PYL receptor polypeptide
mutants identified from second round of shuffling

| Clone # | Mutations Present | SEQ ID NO |
|---|---|---|
| 27B-1 | P42S, K59R, D97N, Y120H, V163I, A172T | 165 |
| 27B-2 | P42S, L44F, K59R, Y120H, V138M, M158I | 166 |
| 27B-3 | P42S, K59R, Y120H, V123I, V139I, M158I | 167 |
| 27B-4 | S47P, V49I, K59R, Y120H, M158I, A177T | 168 |
| 27B-7 | K59R, V81M, Y120C, M158I, V163I | 169 |
| 27B-8 | P42S, K59R, D97N, Y120H, V163I, A172T | 165 |

A third round of shuffling was conducted by combining DNA for the “27B′” mutants shown in Table 7 and ePCR1 library to create a library of ˜150,000 clones which was then transformed into MAV99 pAD-HAB 1. Constitutive mutants were then depleted by growth on FOA after which fenhexamid sensitive mutants were selected by growth on media lacking uracil but containing 1.5 μM fenhexamid, using the methods described above. This effort yielded the following fenhexamid responsive variants (Table 8):

TABLE 8
Fenhexamid-responsive PYR/PYL receptor polypeptide
mutants identified from third round of shuffling

| | | SEQ |
|---|---|---|
| Clone # | Mutations Present | ID NO |
| 27C-1 | P27L, P42S, K59R, D97N, Y120H, M158I, T173A | 170 |
| 27C-2 | P42S, K59R, R74C, Y120H, M158I | 171 |
| 27C-3 | S29N, K59R, D97N, Y120H, V163I, A72T | 172 |
| 27C-5 | P42S, K59R, Y120H, V123I, V139I, M158I, V163I | 173 |
| 27C-16 | K59R, V81M, Y120H, M158I, V163I | 174 |
| 27C-18 | E12K, K59R, V75I, D97N, Y120H, V163I, A172T | 175 |
| 27C-19 | L33F, P42S, K59R, Y120H, V123I, M158I | 176 |
| 27C-20 | P42S, K59R, Y120H, M158I, V163I, V174I | 177 |
| 27C-21 | R10Q, P42S, K59R, D97N, Y120H, V163I, A172T | 178 |

### Establishing the Role of Specific Mutations in Fenhexamid-Responsive Mutant 27C-2

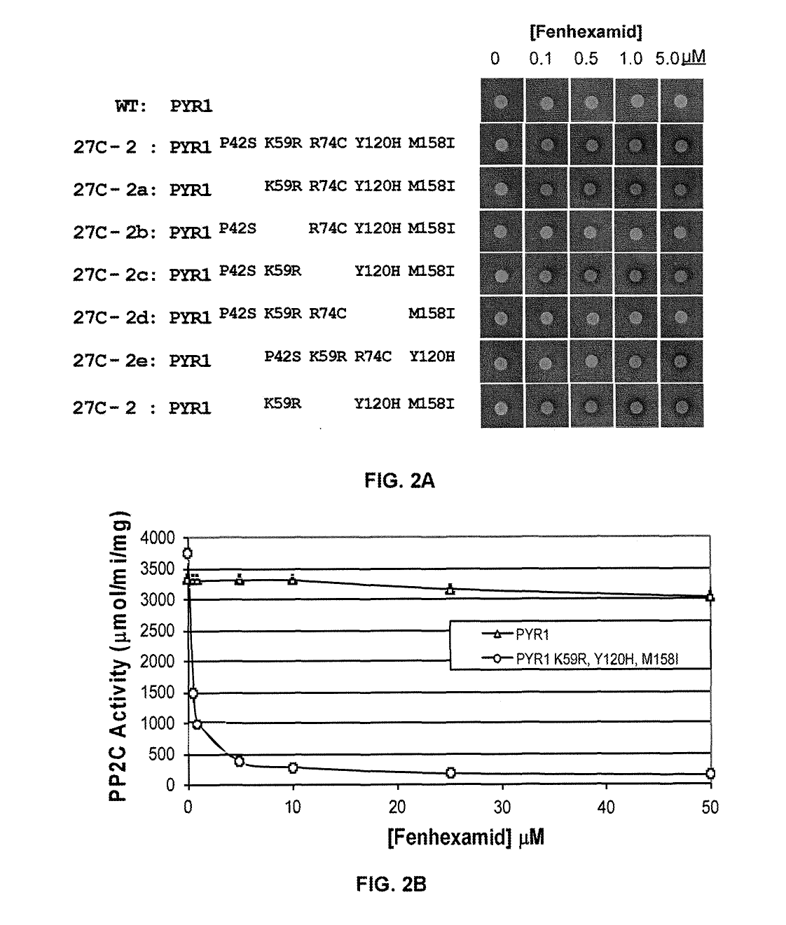

Mutagenesis protocols often introduce spurious mutations that do not affect the desired functionality. To establish the relevance of the residues identified by our screens, we focused on a highly sensitive fenhexamid-responsive mutant identified, 27C-2, which contains 5 mutations (P42S, K59R, R74C, Y120H, and M158I; SEQ ID NO:171; see Table 8) in comparison to the wild-type PYR1 sequence. The presence of K59R, Y120H, and M158I in many other isolated mutants suggested they were likely to be mutations contributing to fenhexamid sensitivity. To probe the role of the 5 mutations in fenhexamid response, each of these residues were reverted back to the wild-type residue using site-directed mutagenesis. The resulting clones were then transformed into the Y190 pAD-HAB1 yeast reporter strain and tested for fenhexamid responsiveness on a range of concentrations. As shown in FIG. 2A, this effort defined K59R, Y120H, and M158I as being sufficient for fenhexamid sensitivity, and established that the mutations P42S and R74C are not sufficient to contribute to fenhexamid sensitivity in the 27C-2 clone. In addition, in vitro receptor assays show that the PYR1^{K59R,Y120H,M158I} triple mutant is sensitive to fenhexamid (IC₅₀ value˜0.4 μM) (FIG. 2B), which demonstrates that the observed sensitivity of the PYR1^{K59R,Y120H,M158I} triple mutant to fenhexamid is not an artifact of the yeast assay system used to identify the triple mutant.

### Engineering Fenhexamid Sensitivity Into PYL2

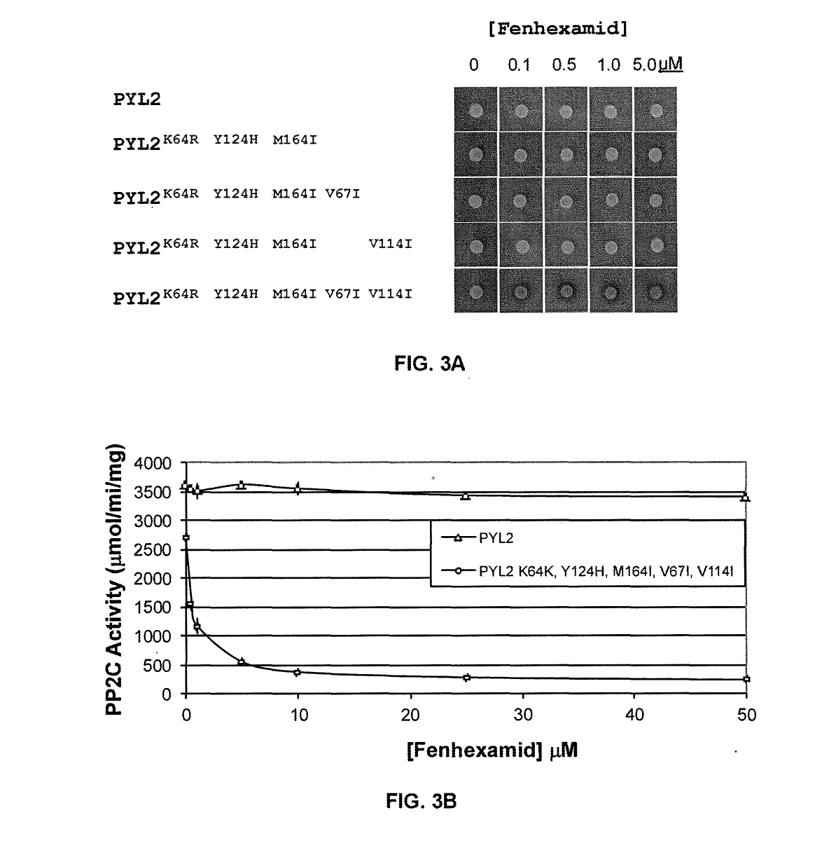

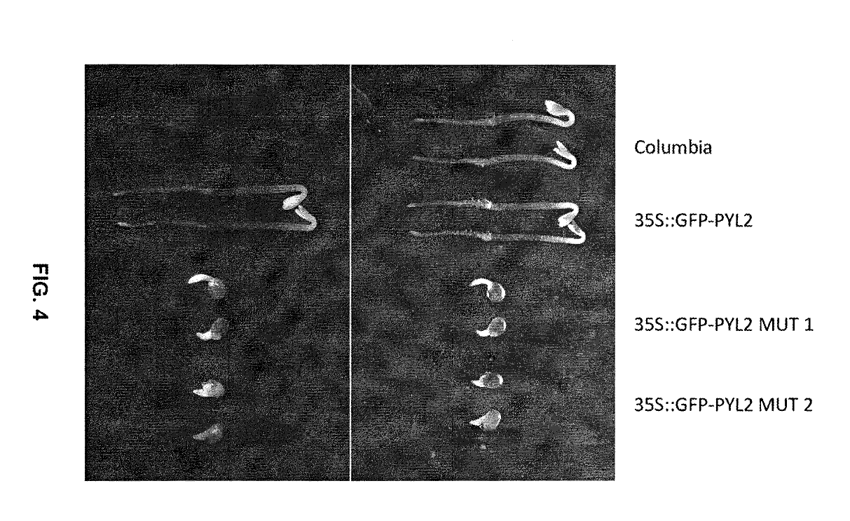

PYR1 is a member of the PYR/PYL receptor protein family, which in Arabidopsis contains 14 members. Moreover, the mutations sufficient for fenhexamid responsiveness in 27C-2 are located in invariant or conserved residues within the ligand binding pocket (K59R, Y120H) or the PYR/PYL-PP2C interface (M158I). Given the conserved nature of these residues, we speculated that fenhexamid sensitivity could be engineered into other receptor family members by mutating homologous residues in other PYR/PYL receptors. To test this, we introduced the homologous mutations in PYL2 (K64R, corresponding to K59R in PYR1; Y124H, corresponding to Y120H in PYR1; and M164I, corresponding to M158I in PYR1), creating the mutant PYL2^{K64R,Y124H,M164I}. As shown in FIG. 3A, this mutant does not show fenhexamid responsiveness when tested using the yeast two-hybrid assay (strain Y190 pAD-HAB1). Recent work (Peterson et al., Nat Struct Mol Biol 17:1109-1113 (2010)) has shown that subtle sequence variations between receptor family members affects a receptor's sensitivity to the selective agonist pyrabactin. In particular, PYR1 and PYL1 show strong responsiveness to pyrabactin, while PYL2 (and other family members) do not. Genetic, biochemical, and structural studies have shown that two key residues in the ligand-binding pocket of PYR1 determine the difference in pyrabactin agonist activity between PYR1 and PYL2. In PYR1, these residues are isoleucines I62 and I110 while in PYL2, the homologous residues (amino acid positions 64 and 114, respectively) are replaced by less bulky valines V67 and V114. Based on the known role of these two residues in affecting differences in ligand responsiveness between receptors, we hypothesized that I62 and I110 may play important roles in fenhexamid response. We therefore introduced the V67I and V1141 mutations (alone or in combination) into the PYL2^{K64R,Y124H,M164I} receptor. The addition of the V67I and V114I mutations together enabled the final mutant receptor PYL2^{K64R,Y124H,M164I,V67I,V114I} to respond to fenhexamid in yeast assays (FIG. 3A) and in vitro PP2C inhibition assays (FIG. 3B).

### Efficacy of Orthogonal Receptors in Plants

To investigate if the fenhexamid response observed in yeast and in vitro experiments would function in plants, transgenic 35S::GFP-PYL2 and 35S::GFP-PYL2^{K64R,Y124H,M164I,V67I,V114I} plants were constructed using standard molecular cloning methods into a modified version of pEGAD (Cutler et al., Proc Natl Acad Sci U.S.A. 97:3718-3723 (2000)) in which a 6×-histidine tag was added onto the N-terminus of GFP. Transgenic plants were made using the floral-dip method and primary transgenics (T1) identified by screening seedlings by epi-fluorescence using a Leica GFP dissecting microscope. The GFP⁺ transgenic plants were grown to maturity and T2 seed collected for further analyses.

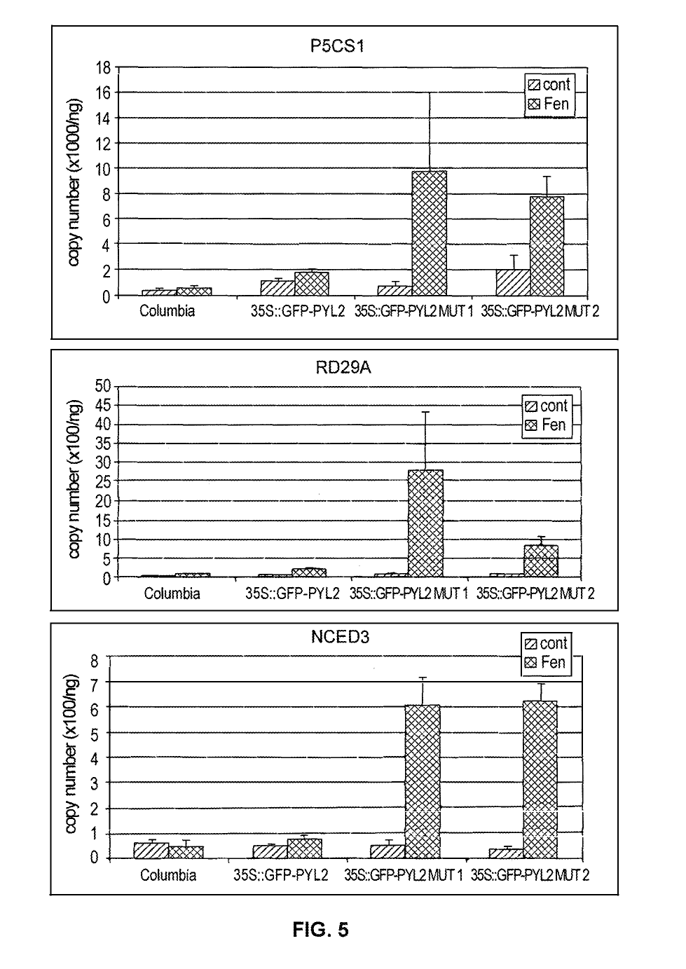

To establish if the fenhexamid responsive PYL2^{K64R,Y124H,M164I,V67I,V114I} mutant functions in planta, T2 segregants and appropriate wild-type control were tested for their ability to grow on 100 μM fenhexamid, as activation of the receptors should inhibit germination. Plants expressing mutant receptors show a strong growth inhibition, suggesting that the ABA pathway is being activated by fenhexamid in plants expressing the mutant, but not the wild-type, receptor (FIG. 4). To further investigate ABA-response activation, we next tested GFP⁺ transgenics (T2 segregants) and appropriate controls for activation of 3 ABA reporter genes (P5CS1, RD29A, and NCED3) in response to exposure to fenhexamid in liquid culture using quantitative RT-PCR methods. As shown in FIG. 5, all three genes show substantial induction by fenhexamid in the two independent PYL2^{K64R,Y124H,M164I,V67I,V67I,V114I} transgenic lines. Thus, genes that are normally ABA regulated in vivo can be activated by fenhexamid using the PYL2^{K64R,Y124H,M164I,V67I,V114I} mutant receptor.

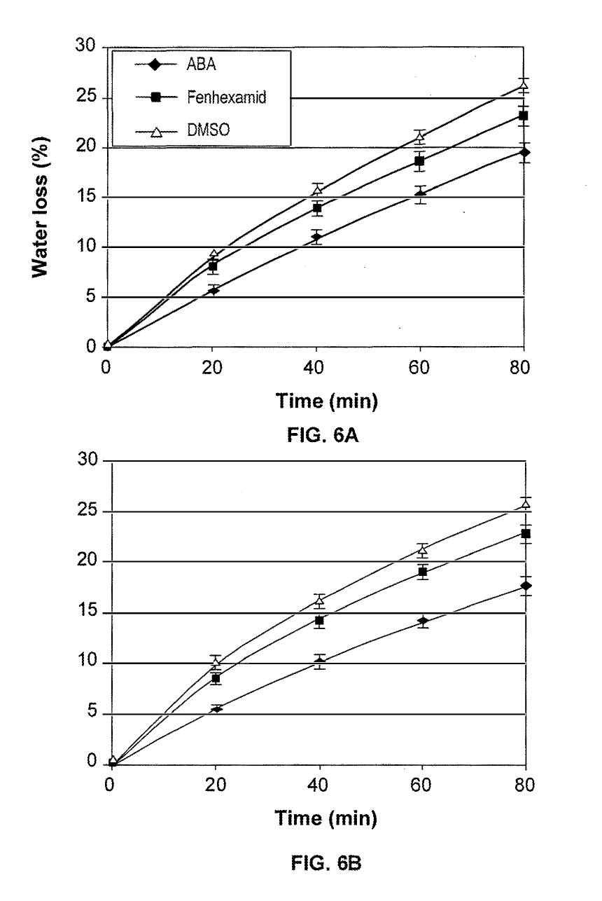

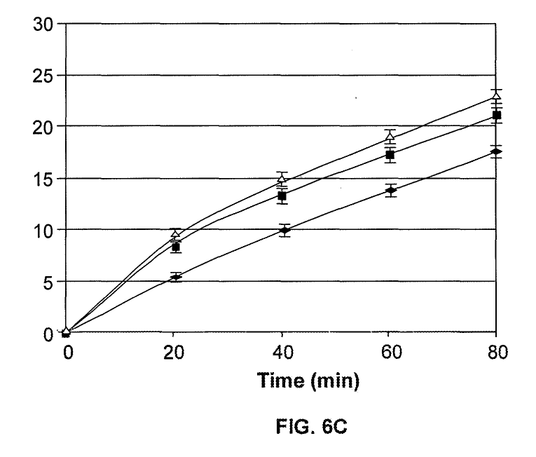

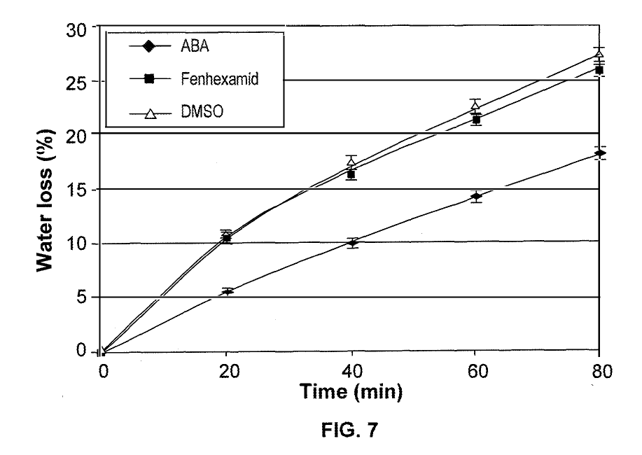

To further establish if the fenhexamid responsive PYL2^{K64R,Y124H,M164I,V67I,V114I} mutant receptor functions in planta, we examined the ability of fenhexamid to reduce water loss in detached leaves of wild-type plants or plants overexpressing PYL2^{K64R,Y124H,M164I,V67I,V114I}. Transgenic or control (wild-type) plants were grown under a 16 hr light/8 hr dark cycle for 3 weeks and then treated with either 100 μM (+)-ABA, 100 μM fenhexamid, or control (containing 0.1% Tween-20, 0.1% DMSO). ABA treatments were conducted as a positive control. The plants were sprayed in the evening prior to conducting water loss experiments. The next morning, approximately 16 hours post-treatment, the above-ground rosettes of the experimental samples were collected and transferred to weighing dishes, 8 plants per measured sample, and maintained under ˜90-100 μEinstein/m² fluorescent light illumination. Four groups of eight plants were measured per time point at 20 min intervals. The experiments were repeated three times over the course of an eight-week interval. In all experiments, fenhexamid pretreatment was sufficient to reduce water loss from PYL2^{K64R,Y124H,M164I,V67I,V114I} transgenic plants (FIG. 6A-C), albeit with less efficacy than ABA. It should be noted, however, that ABA can activate all of the PYR/PYL receptors in a plant genome (at least 13 in Arabidopsis), while fenhexamid is selectively activating a single engineered receptor, PYL2^{K64R,Y124H,M164I,V67I,V114I}. As a control for the effects of fenhexamid, we subjected wild-type plants to treatments with either 100 μM (+)-ABA, 100 μM fenhexamid, or control (containing 0.1% Tween-20, 0.1% DMSO) following the same protocol described above for transgenic plants. These experiments showed that fenhexamid does not affect water loss in wild-type plants (FIG. 7). Thus, the expression of the fenhexamid responsive PYL2^{K64R,Y124HmM164I,V67I,V114I} mutant in transgenic plants enables fenhexamid to activate ABA signaling and physiological response.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A plant comprising a heterologous expression cassette, the expression cassette comprising a promoter operably linked to a polynucleotide encoding a mutated PYR/PYL receptor polypeptide, wherein the mutated PYR/PYL receptor polypeptide is agonized by a chemical when the chemical is contacted to the mutated PYR/PYL receptor polypeptide and wherein the chemical does not significantly agonize a wild-type PYR/PYL receptor polypeptide when the chemical is contacted to the wild-type PYR/PYL receptor polypeptide.

2. The plant of claim 1, wherein the chemical comprises a fungicide, an herbicide, a pesticide, a nematicide, a plant activator, a synergist, an herbicide safener, a plant growth regulator, an insect repellant, or a fertilizer.

3. The plant of claim 1, wherein the chemical is selected from the group consisting of bromoxynil, chloroxynil, ioxynil, coumatetralyl, dichlobenil, fenhexamid, benoxacor, and BTH (acibenzolar-s-methyl).

4. The plant of claim 1, wherein the amino acid of the mutated PYR/PYL receptor polypeptide corresponding to position K59 of SEQ ID NO:1 is X, wherein X is alanine, cysteine, aspartic acid, glutamic acid, phenylalanine, glycine, histidine, leucine, methionine, glutamine, arginine, serine, threonine, valine, tyrosine, asparagine, or tryptophan.

5. (canceled)

6. The plant of claim 4, wherein the mutated PYR/PYL receptor polypeptide further comprises at least one additional mutation at an amino acid corresponding to positions 21, 41, 50, 57, 60, 82, 92, 102, 116, 125, 141, and/or 151 in PYR1 (SEQ ID NO:1) wherein the mutation is selected from H21Y, P41L, R50G, T57A, H60R, 182N, S92T, E102G, R116K, T125A, E141Q, E141D, N151D, or combinations thereof, and wherein the mutated PYR/PYL receptor polypeptide is agonized by bromoxynil, chloroxynil, or ioxynil when the bromoxynil, chloroxynil, or ioxynil is contacted to the mutated PYR/PYL receptor polypeptide.

7. The plant of claim 4, wherein the mutated PYR/PYL receptor polypeptide further comprises at least one additional mutation at an amino acid corresponding to positions 10, 12, 25, 27, 29, 33, 42, 43, 44, 47, 49, 74, 75, 81, 97, 110, 120, 123, 124, 133, 138, 139, 144, 154, 158, 163, 172, 173, 174, and/or 177 in PYR1 (SEQ ID NO:1) wherein the mutation is selected from R10Q, E12G, E12K, L25R, P27L, S29N, L33F, P42S, E43G, L44F, S47P, V49I, R74C, V75I, V81M, D97N, I110S, Y120C, Y120H, V123I, T124M, N133D, V138M, V139I, V144A, E154G, M158I, V1631, A172T, T173A, V174I, and/or A177T or combinations thereof, and wherein the mutated PYR/PYL receptor polypeptide is agonized by fenhexamid when the fenhexamid is contacted to the mutated PYR/PYL receptor polypeptide.

8. The plant of claim 1, wherein the mutated PYR/PYL receptor polypeptide comprises mutations at amino acids corresponding to positions 59, 120, and 158 in PYR1 (SEQ ID NO:1) wherein the mutations are K59R, Y120H, and M158I, and wherein the mutated PYR/PYL receptor polypeptide is agonized by fenhexamid when the fenhexamid is contacted to the mutated PYR/PYL receptor polypeptide.

9. The plant of claim 8, wherein the mutated PYR/PYL receptor polypeptide further comprises isoleucine residues at the amino acid positions corresponding to positions 62 and 110 in PYR1 (SEQ ID NO:1).

10. The plant of claim 4, wherein the mutated PYR/PYL receptor polypeptide further comprises at least one additional mutation at an amino acid corresponding to positions 27 and/or 63 in PYR1 (SEQ ID NO:1) wherein the mutation is selected from P27L, K63N, or combinations thereof, and wherein the mutated PYR/PYL receptor polypeptide is agonized by dichlobenil when the dichlobenil is contacted to the mutated PYR/PYL receptor polypeptide.

11. The plant of claim 1, wherein the mutated PYR/PYL receptor polypeptide comprises at least one mutation at an amino acid corresponding to positions 26, 37, 71, and/or 94 in PYR1 (SEQ ID NO:1) wherein the mutation is selected from D26G, R37Q, F71S, E94D, or combinations thereof, and wherein the mutated PYR/PYL receptor polypeptide is agonized by dichlobenil when the dichlobenil is contacted to the mutated PYR/PYL receptor polypeptide.

12. The plant of claim 4, wherein the mutated PYR/PYL receptor polypeptide further comprises a mutation at an amino acid corresponding to position 119 in PYR1 (SEQ ID NO:1) wherein the mutation is N119Y, and wherein the mutated PYR/PYL receptor polypeptide is agonized by benoxacor when the benoxacor is contacted to the mutated PYR/PYL receptor polypeptide.

13. The plant of claim 1, wherein the mutated PYR/PYL receptor polypeptide comprises at least one mutation at an amino acid corresponding to positions 110, 114, and/or 138 in PYR1 (SEQ ID NO:1) wherein the mutation is selected from I110T, E114D, V138M, or combinations thereof, and wherein the mutated PYR/PYL receptor polypeptide is agonized by benoxacor when the benoxacor is contacted to the mutated PYR/PYL receptor polypeptide.

14. The plant of claim 4, wherein the mutated PYR/PYL receptor polypeptide further comprises at least one mutation at an amino acid corresponding to positions 24, 82, 159, and/or 161 in PYR1 (SEQ ID NO:1) wherein the mutation is selected from Q24R, I82T, F159L, D161G, or combinations thereof, and wherein the mutated PYR/PYL receptor polypeptide is agonized by BTH (acibenzolar-s-methyl) when the BTH is contacted to the mutated PYR/PYL receptor polypeptide.

15. The plant of claim 1, wherein the mutated PYR/PYL receptor polypeptide comprises at least one mutation at an amino acid corresponding to positions 115 and/or 159 in PYR1 (SEQ ID NO:1) wherein the mutation is selected from H115Y, F159S, F159L, or combinations thereof, and wherein the mutated PYR/PYL receptor polypeptide is agonized by BTH (acibenzolar-s-methyl) when the BTH is contacted to the mutated PYR/PYL receptor polypeptide.

16-22. (canceled)

23. A plant cell, seed, flower, leaf, or fruit from the plant of claim 1.

24. (canceled)

25. A method of improving abiotic stress tolerance in the plant of claim 1 by contacting the plant with a chemical selected from the group consisting of bromoxynil, chloroxynil, ioxynil, coumatetralyl, dichlobenil, fenhexamid, benoxacor, and BTH (acibenzolar-s-methyl).

26. A method of making a mutated PYR/PYL receptor polypeptide that is agonized by a chemical when the chemical is contacted to the mutated PYR/PYL receptor polypeptide, wherein the chemical does not significantly agonize a wild-type PYR/PYL receptor polypeptide when the chemical is contacted to the wild-type PYR/PYL receptor polypeptide, the method comprising
(a) mutagenizing the wild-type PYR/PYL receptor polypeptide;
(b) contacting one or more mutated PYR/PYL receptor polypeptides with the chemical; and
(c) determining whether the chemical activates the one or more mutated PYR/PYL receptor polypeptides, wherein activation identifies the one or more mutated PYR/PYL receptor polypeptides as being agonized by the chemical.

27. The method of claim 26, further comprising, prior to step (b), screening the chemical to determine whether the chemical binds to the wild-type PYR/PYL receptor polypeptide prior to contacting the one or more mutated PYR/PYL receptor polypeptides with the chemical.

28. An expression cassette comprising a promoter operably linked to a polynucleotide encoding a mutated PYR/PYL receptor polypeptide, wherein the mutated PYR/PYL receptor polypeptide is agonized by a chemical when the chemical is contacted to the mutated PYR/PYL receptor polypeptide and wherein the chemical does not significantly agonize a wild-type PYR/PYL receptor polypeptide when the chemical is contacted to the wild-type PYR/PYL receptor polypeptide.

29-30. (canceled)

31. A polypeptide comprising a mutated PYR/PYL receptor polypeptide that is at least 70% identical to any of SEQ ID NOs:124-148 or 164-178.

32. (canceled)
